(19) United States
(12) Patent Application Publication
(10) Pub. No.: US 2023/0323294 A1
(43) Pub. Date: Oct. 12, 2023

(54) DIFFERENTIATION OF CORTICAL NEURONS FROM HUMAN PLURIPOTENT STEM CELLS

(71) Applicant: MEMORIAL SLOAN-KETTERING CANCER CENTER, New York, NY (US)

(72) Inventors: Yuchen Qi, New York, NY (US); Lorenz Studer, New York, NY (US)

(73) Assignee: MEMORIAL SLOAN-KETTERING CANCER CENTER, New York, NY (US)

(21) Appl. No.: 18/325,690

(22) Filed: May 30, 2023

Related U.S. Application Data
(63) Continuation of application No. 16/047,393, filed on Jul. 27, 2018, now abandoned.
Application No. 16/047,393 is a continuation of application No. PCT/US2017/015480, filed on Jan. 27, 2017.
(60) Provisional application No. 62/449,488, filed on Jan. 23, 2017.
(60) Provisional application No. 62/287,821, filed on Jan. 27, 2016.

(51) Int. Cl. C12N 5/0793; A61P 25/28; A61K 35/30

(52) U.S. Cl. CPC C12N 5/0619; A61P 25/28; A61K 35/30; C12N 2501/42; C12N 2506/03; C12N 2501/415; C12N 2501/15; C12N 2501/999; C12N 2506/02; C12N 2501/155

(57) ABSTRACT

The presently disclosed subject matter provides for in vitro methods of inducing differentiation of human stem cells into cortical neurons, and cortical neurons generated by such methods. The presently disclosed subject matter also provides for uses of such cortical neurons for treating neurodegenerative CNS disorders.

### CROSS REFERENCE TO RELATED APPLICATIONS

The present application is a Continuation of U.S. patent application Ser. No. 16/047,393, filed Jul. 27, 2018, which is a Continuation of International Patent Application No. PCT/US17/015480, filed Jan. 27, 2017, which claims priority to U.S. Provisional Application No. 62/287,821 filed Jan. 27, 2016, and U.S. Provisional Application No. 62/449,488 filed Jan. 23, 2017, the contents of each of which are incorporated by reference in their entireties herein, and priority to each of which is claimed.

### GRANT INFORMATION

This invention was made with government support under grant numbers R01NS072381 and NS084334 awarded by the National Institutes of Health. The government has certain rights in the invention.

### SEQUENCE LISTING

The specification further incorporates by reference the Sequence Listing submitted herewith via EFS on May 30, 2023. Pursuant to 37 C.F.R. § 1.52(e)(5), the Sequence Listing xml file, identified as 0727341452SL.xml, is 21,719 bytes and was created on May 30, 2023. The Sequence Listing, electronically filed herewith, does not extend beyond the scope of the specification and thus does not contain new matter.

### 1. INTRODUCTION

The presently disclosed subject matter relates to cortical neurons, and precursors thereof, derived from human stem cells, and their use in cell-based treatment of neurological disorders.

### 2. BACKGROUND OF THE INVENTION

Over the last few years, methods have been developed to convert human pluripotent stem cells (hPSCs) into early neural lineages. A particularly efficient strategy is the use of small molecules inhibiting SMAD signaling (e.g., dual SMAD inhibition) to trigger differentiation of human embryonic stem cells (hESCs) or human induced pluripotent stem cells (hiPSCs) into PAX6+ central nervous system (CNS) neural precursors within 11 days of differentiation¹. Neural subtype specification can be further modulated using additional small molecules targeting pathways such as WNT signaling. Timed exposure to compounds activating WNT signaling under dual-SMAD inhibition conditions induces SOX10+ neural crest lineages. In contrast, inhibition of WNT signaling enhances the induction of FOXG1+ forebrain precursors²⁻⁴. While those manipulations efficiently specify defined neural precursor cell populations, further differentiation into functional neurons has been a lengthy process that can extend over weeks if not months. In an effort to accelerate neuronal fate acquisition the use of two additional small molecules was described: SU5402, a potent inhibitor of fibroblast growth factor (FGF) signaling⁵ and DAPT, a γ-secretase inhibitor blocking Notch signaling⁶. The combinatorial application of those two inhibitors (SD) with dual SMAD inhibition and WNT activation yields 75% post-mitotic neurons by day 11 of differentiation, the same time period required for neural precursor cell induction under standard dual-SMAD inhibition conditions¹. However, co-expression of BRN3A and ISL1 in those rapidly-induced neurons defined them as peripheral sensory rather than PAX6-derived CNS neurons. Therefore it has remained unclear whether strategies to accelerate neuronal fate acquisition during sensory fate specification can be adapted for CNS fates. PAX6-derived cortical neurons are of relevance in the areas of human development and neurodegenerative CNS disorders. Therefore, there is a need in the art for methods and compositions for rapid induction of cortical neurons.

### 3. SUMMARY OF THE INVENTION

The presently disclosed subject matter relates to cortical neurons, and precursors thereof, for example proximate precursors thereof, derived from stem cells, e.g. by in vitro differentiation.

The present invention is based, at least in part, on the discovery that inhibition of MAPK/ERK kinase accelerates the differentiation of cortical neurons from stem cells contacted with (i) one or more inhibitor of transforming growth factor beta (TGFβ)/Activin-Nodal signaling; (ii) one or more inhibitor of bone morphogenetic protein (BMP) signaling; (iii) one or more inhibitor of Wnt signaling; (iv) one or more inhibitor of FGF signaling; and (v) one or more inhibitor of Notch signaling.

In certain embodiments, the in vitro method for inducing differentiation of human stem cells into cortical neurons (and proximate precursors thereof) comprises contacting a population of human stem cells with (i) an effective amount of one or more inhibitor of transforming growth factor beta (TGFβ)/Activin-Nodal signaling, (ii) an effective amount of one or more inhibitor of bone morphogenetic protein (BMP) signaling, and (iii) an effective amount of one or more inhibitor of wingless (Wnt) signaling, wherein the cells are contacted with effective amounts of the inhibitors for at least 4 days, at least 5 days, at least 6 days, at least 7 days, at least 8 days, at least 9 days, at least 10 days; or for up to 4 days, for up to 5 days, for up to 6 days, for up to 7 days, for up to 8 days, for up to 9 days, or for up to 10 days.

In certain embodiments, the method further comprises contacting the cells with (iv) an effective amount of one or more inhibitor of MAPK/ERK kinase signaling (also known as MEK), (v) an effective amount of one or more inhibitor of FGF signaling, and (vi) an effective amount of one or more inhibitor of Notch signaling. In certain embodiments, the cells are contacted with (iv), (v) and/or (vi) for at least 4 days, for at least 5 days, for at least 6 days, for at least 7 days, for at least 8 days, for at least 9 days, for at least 10 days, for at least 11 days, for at least 12 days, for at least 13 days, for at least 14 days, or at least up to 4 days, up to 5 days, up to 6 days, up to 7 days, up to 8 days, up to 9 days, up to 10 days, up to 11 days, up to 12 days, up to 13 days or up to 14 days. In certain embodiments, the cells are initially contacted with effective amounts of the (iv), (v) and/or (vi) inhibitors at least 2 days (or at least 48 hours) after the cells are initially contacted with an effective amount of the one or more inhibitor of TGFβ/Activin-Nodal signaling.

In certain embodiments, the cells are initially contacted with an effective amount(s) of the (iv), (v) and/or (vi) inhibitor about 1, about 2, about 3, about 4, about 5, or about 6 days after the cells are initially contacted with effective amounts of (i), (ii) and (iii) inhibitors. In certain embodiments, the cells are initially contacted with an effective amount(s) of the (iv), (v) and/or (vi) inhibitor about 24 hours, about 48 hours, about 72 hours, about 96 hours, about 120 hours, or about 144 hours after the cells are initially contacted with effective amounts of (i), (ii) and (iii) inhibitors.

In certain embodiments, the human stem cells are contacted with effective amounts of inhibitors (i) through (iii) for at least about 2, 3, 4, 5, 6, 7, 8, 9, or 10 days, and are contacted with effective amounts of inhibitors (iv) through (vi) for at least about 2, 3, 4, 5, 6, 7, 8, 9, or 10 days, and are then further contacted for at least about 2, 3, 4, 5, 6, 7, 8, 9, or 10 days with: an effective amount of one or more inhibitor of Notch signaling; an effective amount of one or more inhibitor of FGF signaling; an effective amount of one or more inhibitor of MAPK/ERK signaling; and/or an effective amount of one or more activator of Wnt signaling

In certain embodiments, the human stem cells are contacted with inhibitors (i) through (iii) for at least about 2, 3, 4, 5, 6, 7, 8, 9, or 10 days, and are contacted with effective amounts of inhibitors (iv) through (vi) for at least about 2, 3, 4, 5, 6, 7, 8, 9, or 10 days, and are then further contacted for at least about 2, 3, 4, 5, 6, 7, 8, 9, or 10 days with one or more inhibitor of Notch signaling.

In certain embodiments, the cells are contacted with effective concentrations of (i) to (vi) for a period of time such that the cells express detectable levels of PAX6. In certain embodiments, said period of time is about 6 days after the cells are initially contacted with an effective concentration of inhibitor (i), i.e., the one or more inhibitor of TGFβ/Activin-Nodal signaling.

In certain embodiments, the cells are contacted with effective concentrations of (i) to (vi) for a period of time such that the cells express detectable levels of TUJ1. In certain embodiments, the cells are contacted with effective concentrations of (i) to (vi) for a period of time such that at least 30% of the cells express detectable levels of TUJ1. In certain embodiments, said period of time is about 13 days after the cells are initially contacted with an effective concentration of inhibitor (i), i.e., the one or more inhibitor of TGFβ/Activin-Nodal signaling. In certain embodiments, the cells further coexpress TBR1 and/or TLE4.

In certain embodiments, the cells are contacted with effective concentrations of (i) to (vi) for a period of time such that the cells express detectable levels of TUJ1 and one or both of TBR1 and/or TLE4.

In certain embodiments, the cells contacted according to the methods described herein express detectable levels of TUJ1, wherein at least 10%, 20%, 30%, 40%, 50%, 60%, 70%, 80%, 90% or more of the cells also expresses detectable levels of TBR1, TLE4, or a combination thereof.

In certain embodiments, the cells are contacted with effective concentrations of (i) to (vi) for a period of time such that the cells express detectable levels of a cortical neuron marker. In certain embodiments, the cortical neuron marker is selected from the group consisting of TB R1, TLE4, DCX, REELIN, CTIP2, SATB2, FOXP2, RGS4, CUX2, BLBP and combinations thereof. In certain embodiments, the cells express a detectable level of a cortical neuron marker after at least 4, 5, 6, 7, 8, 9, 10, 11, 12, 13, 14, 15, 20, 25, 30, 33 days or more after initially being contacted with an effective concentration of inhibitor (i), i.e., the one or more inhibitor of TGFβ/Activin-Nodal signaling.

In certain embodiments, the cells prepared according to the methods described herein exhibit electrophysiological activity of differentiated cortical neurons at least 16 days after being contacted with an effective concentration of (i), i.e., the one or more inhibitor of transforming growth factor beta (TGFβ)/Activin-Nodal signaling.

In certain embodiments, the in vitro method for inducing differentiation of human stem cells into cortical neurons and precursors thereof comprises contacting a population of human stem cells with effective concentrations of (i) one or more inhibitor of transforming growth factor beta (TGFβ)/Activin-Nodal signaling, (ii) one or more inhibitor of bone morphogenetic protein (BMP) signaling, (iii) one or more inhibitor of Wnt signaling, (iv) one or more inhibitor of MAPK/ERK kinase signaling, (v) one or more inhibitor of FGF signaling, and (vi) one or more inhibitor of Notch signaling. In certain embodiments, the cells are contacted with (iv), (v) and (vi) at least 2 days or at least 3 days after contacting the cells with (i), (ii) and (iii). In certain embodiments, the cells are cultured for at least between 4 and 20 days, or at least between 6 and 16 days, at least between about 8 and 14 days, or at least between about 10 and 12 days after initially being contacted with an effective concentration of (i), i.e., the one or more inhibitor of TGFβ/Activin-Nodal signaling.

In certain embodiments, the method comprises (a) initially contacting human pluripotent stem cells with effective concentrations of (i) one or more inhibitor of transforming growth factor beta (TGFβ)/Activin-Nodal signaling, (ii) one or more inhibitor of bone morphogenetic protein (BMP) signaling, (iii) one or more inhibitor of Wnt signaling; (b) culturing said cells, for at least about six or seven days, with effective concentrations of (i) one or more inhibitor of transforming growth factor beta (TGFβ)/Activin-Nodal signaling, (ii) one or more inhibitor of bone morphogenetic protein (BMP) signaling, (iii) one or more inhibitor of Wnt signaling; (c) initially contacting said cells, at least about two or three days after (a), with effective concentrations of (iv) one or more inhibitor of MAPK/ERK kinase signaling, (v) one or more inhibitor of FGF signaling, and (vi) one or more inhibitor of Notch signaling; and (d) culturing said cells, for at least about ten or eleven days or until at least 20% of said cells express TUJ1, with effective concentrations of (iv) one or more inhibitor of MAPK/ERK kinase signaling, (v) one or more inhibitor of FGF signaling, and (vi) one or more inhibitor of Notch signaling.

In certain embodiments, the method further comprises subjecting said population of differentiated cells to conditions favoring maturation of said differentiated cells into a population of cortical neurons. In certain embodiments, said conditions favoring maturation comprise culturing said population of differentiated cells in a suitable cell culture medium. In certain embodiments, said conditions favoring maturation comprise contacting said population of differentiated cells with one or more molecule that enhances maturation of said precursors into cortical neurons. In certain embodiments, said one or more molecule that enhances maturation are selected from the group consisting of activators of brain derived neurotrophic factor (BDNF), cAMP, and ascorbic acid signaling. In certain embodiments, the cells are contacted with said maturation factors at least, or up to, 5, 6, 7, 8, 9, 10 or 12 days after initially being contacted with an effective concentration of (i), i.e., the one or more inhibitor of TGFβ/Activin-Nodal signaling.

The present disclosure also provides for a population of in vitro differentiated cells expressing one or more neuronal marker, for example, a cortical neuron marker, or precursor cells thereof, prepared according to the methods described herein. In certain embodiments, at least about 70% (e.g., at least about 75%, at least about 80%, at least about 85%, at least about 90%, at least about 95%, or at least about 99%, or at least about 99.5%) of the population of cells express one or more cortical neuron marker and wherein less than about 15% (e.g., less than about 10%, less than about 5%, less than about 4%, less than about 3%, less than about 2%, less than about 1%, less than about 0.5%, or less than about 0.1%) of the population of cells express one or more marker selected from the group consisting of stem cell markers (e.g., OCT4, NANOG, SOX2, LIN28, SSEA4 and/or SSEA3), glial cell markers (e.g., GFAP, AQP4, and/or OLIG2), retinal cell markers (e.g., CHX10), peripheral sensory neurons (e.g., BRN3A, and/or ISL1), neural crest precursors (e.g., SOX10), or cranial placode precursors (e.g., SIX1).

In certain embodiments, the differentiated cell population is derived from a population of human stem cells. The presently disclosed subject matter further provides for compositions comprising such differentiated cell population.

Furthermore, the presently disclosed subject matter provides for kits for inducing differentiation of stem cells. In certain embodiments, the kit comprises one or more of the following: (a) one or more inhibitor of transforming growth factor beta (TGFβ)/Activin-Nodal signaling, (b) one or more inhibitor of BMP signaling, (c) one or more inhibitor of Wnt signaling (d) one or more inhibitor of FGF signaling, (e) one or more inhibitor of Notch signaling, (f) one or more inhibitor of MAPK/ERK kinase signaling, and (g) instructions for inducing differentiation of the stem cells into a population of differentiated cells that express one or more neuronal marker, for example, a cortical neuron marker, or precursor cells thereof.

The presently disclosed subject matter also provides for kits comprising a population of differentiated cells that express one or more neuronal marker, for example, a cortical neuron marker, or precursor cells thereof, wherein the cells are prepared according to the methods described herein.

In certain embodiments, said human stem cells are selected from the group consisting of human embryonic stem cells, human induced pluripotent stem cells, human parthenogenetic stem cells, primordial germ cell-like pluripotent stem cells, epiblast stem cells, and F-class pluripotent stem cells.

The presently disclosed subject matter further provides for methods of treating a neurodegenerative disorder in a subject. In certain embodiments, the method comprises administering an effective amount of the differentiated cell population described herein into a subject suffering from a neurodegenerative disorder.

The presently disclosed subject matter further provides for a differentiated cell population described herein for treating a neurodegenerative disorder in a subject.

The presently disclosed subject matter further provides for uses of the differentiated cell population described herein in the manufacture of a medicament for treating a neurodegenerative disorder.

In certain embodiments, the neurodegenerative disorder is Parkinson's disease, Alzheimer's disease, or schizophrenia.

### 4. BRIEF DESCRIPTION OF THE DRAWINGS

FIGS. 1A-1I depict illustrations, images and graphs showing the rapid induction of cortical neurons from human pluripotent stem cells using a combinatorial small molecule-based protocol. FIG. 1A is a schematic illustration of the pathway manipulations to generate neural crest versus forebrain fates and neural precursor versus accelerated neuronal fates. Protocols for the derivation of SOX10+ neural crest precursor, PAX6+ CNS precursors and BRN3A/ISL1+sensory neurons have been reported while the current invention is focused on a strategy for the rapid induction of cortical neurons. FIG. 1B depicts images depicting the validation of the PAX6::H2B-GFP, SOX10::GFP and SIX1::H2B-GFP hESC-based reporter lines by assessing co-labeling with matched protein marker. FIG. 1C depicts graphs of the time-course quantitative analyses of CNS (PAX6::H2B-GFP, left), NC (SOX10::GFP, middle) and placode (SIX1::H2B-GFP, right) induction under the various protocols. (LSB=dual SMAD inhibition; XAV=XAV939, tankyrase inhibitor (WNT-i); CHIR=CHIR99021, GSK313-inhibition (WNT-activation). S=SU5402, FGFR inhibitor; D=DAPT, γ-secretase inhibitor (Notch-i). N=3 independent batches of cell cultures per each condition and cell line. FIG. 1D depicts immunocytochemistry images for the CNS precursor marker PAX6, the neural crest precursor marker SOX10 and the neuronal marker TUJ1 during CNS versus NC based hPSC differentiation. FIG. 1E depicts immunohistochemistry images of the CNS and NC based hPSC differentiation in combination with S/D exposure to accelerate neuronal fate acquisition. FIG. 1F is a graph showing the quantification of the TUJ1 data from FIGS. 1D and 1E at day 13 of differentiation by intracellular flow. Data in FIGS. 1D and 1E are derived from 3 or 5 independent experiments per condition and cell line. Black dots represent values from independent experiments. From left to right, N=3, 3, 5, 3. Statistics was done using unpaired t test with Welch's correction (two-tailed). XAV vs. XAV+S/D: t=9.510 dF=2.160, P=0.0084. CHIR+S/D vs. XAV+S/D: t=13.40, df=4.718, P<0.0001. FIG. 1G depicts graphs of the P and S single and combinatorial dose response analyses based on quantification of the percentages of NESTIN+ and TUJ1+ cells by intracellular flow cytometry (upper), and an estimate of the total neurons in culture per cm² (for every hPSC plated at day 0 under P1S5D and P8S10D conditions, ˜0.7 and 0.2 neurons were obtained respectively at day 13). P=PD0325901, ERK/MEK inhibitor. The digits following P, S, D represent the respective concentrations in μM. Each column represents N=2 technical replicates per marker screened. FIG. 1H depicts immunocytochemistry images for PAX6/TUJ1 in P1S5D and P8S10D cultures by day 13. FIG. 1I depicts a graph showing a quantification of the TUJ1+ cells by intracellular flow cytometry at day 13. Black dots represent values from independent experiments. From left to right, N=4, 5, 5, 4. Statistics was done using unpaired t test with Welch's correction (two-tailed). XAV+P(1)/D vs. XAV+P(1)/S(5)/D: t=4.057, dF=6.045, P=0.0066. XAV+P(1)/S(5) vs. XAV+P(1)/S(5)/D: t=20.62, dF=4.880, P<0.0001. XAV+P(1)/S(5)/D vs. XAV+P(8)/S(10)/D: t=13.26, dF=6.397, P<0.0001. Scale bars: 100 μm (b), 200 μm (d, e, h). Error bars represent s. e. m. ** P<0.01, **** P<0.0001.

FIGS. 2A-2D depict illustrations, images and graphs showing the rapid cortical neuronal induction in the hESC and hiPSC lines. FIG. 2A depicts an illustration showing an in vitro differentiation scheme of the P/S/D protocol. hPSCs were plated one day prior to differentiation at 200,000/cm² in hESC media supplemented with 50 ng/ml FGF2 and 10 μM ROCK-Inhibitor Y-27632. Small molecules are added in the presence of dual-SMAD inhibition (LDN193189, SB431542) and XAV939 treatment (LSBX). Optimized timing for the application of PD0325901, SU5402 and DAPT (P/S/D) are shown. FIGS. 2B and 2C depict images showing the validation of the (b) P1S5D and (c) P8S10D protocols on the hiPSC lines by immunofluorescence of the TBR1/TUJ1 expression at day 13. FIG. 2D depicts a graph showing the quantification of the neuron induction efficiency at day 13 by intracellular flow cytometry for the various hiPSC lines tested. N=3 independent batches of cell cultures for line 1.1, 1.2, 1.3, and N=1 for line 7.1, 7.2 and 7.4. Scale bars: 50 inn. Error bars represent s. e. m.

FIGS. 3A-3L depict graphs and images of the temporal and phenotypic characterization of hPSC derived neurons by day 13 of differentiation. FIG. 3A depicts a graph showing a time-course analysis of neuronal induction efficiency by intracellular flow cytometry (N=4 independent batches of cell cultures), unpaired t test with Welch's correction (two-tailed) to compare mean difference between each group at day 13. LSBX vs. P1S5D: t=20.79 dF=3.042, P=0.0002. 3i vs. P1S5D: t=0.8277 dF=5.013, P=0.4455, N.S.. P1S5D vs. P8S10D: t=12.79 dF=5.994, P<0.0001. FIG. 3B depicts graphs of the time-course qRT-PCR analysis at day 5, 8 11, 13 of differentiation. OCT4(POU5F1): Human pluripotency marker; PAX6: Dorsal cortical progenitor marker; FOXG1: Forebrain marker; DCX: Pan-neuron marker; TBR1: preplate, subplate and cortical Layer VI neuron marker; REELIN: cortical Layer I (Cajal-Retzius cell) neuron marker. FC: fold change. N=3 independent batches of cell cultures. FIG. 3C depicts images showing the TBR1/TUJ1 expression by immunofluorescence at day 13. FIGS. 3D and 3E depict graphs showing the quantification of (D) the percentage of TBR1+ cells among total cells (t=3.151, dF=7.820, P=0.0140, two-tailed), or (E) among TUJ1+ neurons (t=1.094, dF=9.997, P=0.2994, two-tailed, N.S.). Black dots represent values from quantification of uniform random selection of six 150 μm×150 μm areas from 3 independent batches of cell cultures. Statistics was done using unpaired t test with Welch's correction. FIG. 3F depicts an illustration of long-term culture protocols beyond day 13. In vitro differentiation before day 8 is the same as described in FIG. 2A. For long-term culture, P1S5D and P8S10D cells were passaged at day 8 of differentiation at 150,000/cm2 and 300,000/cm2 respectively in NB/B27+BCA medium without adding inhibitors thereafter. Cells were fixed at various time points and processed for immunocytochemistry or RNA extraction and qRT-PCR analysis. FIG. 3G shows that long-term maintenance of P1S5D and P8S10D cells produced neurons constituting distinct cortical layer fates: FOXP2 (layer V-VI), TLE4 (layer VI), CTIP2 (layer V), SATB2 (layer II-III, V), RGS4 (layer II-III, layer V), CUX2 (cortical progenitors and layer II-IV). FIG. 3H depicts a graph showing the quantification of TBR1+, CTIP2+ and SATB2+ cells in total cell population using P1S5D differentiation. N=quantification of 6 randomly selected photo frames captured using a 20× objective from 2 independent batches of cell cultures. FIG. 3I depicts a graph showing quantification of the percentage of EdU positive among marker positive cells at day 40 of P1S5D differentiation. EdU was added to the cultures for 48 hrs at various time points of differentiation (as indicated on x-axis) and the cultures were fixed at day 40. Colored dots represent values of quantification results of individual photo frames from 2 independent batches of cell cultures. From left to right, N=3, 4, 4; 0, 0, 0; 3, 4, 3; 3, 3, 4; 3, 5, 1; 1, 3, 4. Scale bars: 50 μm. Error bars represent s. e. m. FIG. 3J depicts representative image showing co-labeling of EdU with TBR1 and CTIP2 at day 40 of P1S5D differentiation. FIG. 3K depicts an alternative scheme of accelerated neuronal differentiation protocol using P/S/D in E6 medium. FIG. 3L depicts validation of P/S/D protocol in E6 by immunocytochemistry of PAX6/TUJ1 and TBR1/TUJ1 expression at day 13. Scale bars: 50 m, except for 100 μm in left and middle panels of FIG. 3L. Error bars represent s. e. m. ** P<0.01, *** P<0.001, **** P<0.0001. N.S.: not significant.

FIG. 4 depicts graphs showing intracellular flow analyses for TUJ1 for the various culture protocols. Each graph depicts the gating of the intracellular flow cytometry of TUJ1+ neuron population at day 13 (blue: TUJ1 stain. red: isotype control).

FIGS. 5A-5B show images and graphs showing the impact of various small molecule-based manipulations in cell signaling on FOXG1 expression. FIG. 5A depicts immunocytochemistry images for FOXG1/TUJ1 co-expression at day 13. FIG. 5B depicts a graph showing a quantification of the FOXG1 transcript expression level by qRT-PCR under P1S5D conditions, but after systematic removal of one of the small molecule factors each. N=3 independent batches of cell cultures. Scale bars: 100 μm. Error bars represent s. e. m.

FIGS. 6A-6F depict illustrations and graphs showing that accelerated induction yields hPSC-derived cortical neurons with mature electrophysiological properties in vitro. FIG. 6A depicts a schematic illustration of six different treatment conditions for neuronal induction and maintenance of neurons. In vitro differentiation protocol up to day 8 is the same as described in FIG. 2A. P1S5D and P8S10D cells were then passaged at 150,000/cm² and 300,000/cm² respectively at day 8 in NB/B27+BCA. Both passaged P1S5D and P8S10D cells were further maintained in three conditions: without inhibitors (+none), with DAPT (+D), and with PD0315901 (1 uM), SU5402 (5 uM), DAPT, CHIR99021 (3 uM) (+PSDC), making 6 different treatments of cells in total. FIG. 6B depicts graphs of representative action potential firing traces at day 16 of differentiation from cells representing the 6 differentiation conditions. FIG. 6C Quantification of auto-firing electrophysiological properties at day 16 (without current injection). Black dots represent values of individual cells. From left to right, N=15, 18, 11, 16, 17, 12 from 4 batches of independent cell cultures. PSDC: P(1)S(5)D+CHIR. FIG. 6D depicts quantification of percentage of cells with indicated firing frequencies at day 16 of differentiation without current injection, and with −10 pA current injection. Injecting −10 pA current triggered evoked firing and enabled an even larger proportion of cells to adopt high firing frequencies. From left to right, N=15, 18, 11, 16, 17, 12 from 4 batches of independent cell cultures. PSDC: P(1 μM) S(5 μM) D+CHIR. FIG. 6E depicts a graph showing voltage-dependent sodium channel responses of P1S5D+none neurons at day 37 by whole-cell patch clamp, which could be blocked by Tetrodotoxin (TTX) that specifically blocks the sodium channel. Inset: protocol used for triggering sodium channel currents. FIG. 6F depicts a graph showing spontaneous excitatory postsynaptic currents (sEPSCs) recorded under P1S5D+none conditions at day 40 indicative of functional synapse formation. sEPSCs could be blocked by NBQX that selectively blocks AMPA receptors indicating excitatory synaptic currents. Error bars represent s. e. m.

FIGS. 7A-7C depict illustrations and graphs summarizing the electrophysiological parameters for P1S5D cultures maintained in the absence of small molecule factors. FIG. 7A is a schematic illustration of the P1S5D+none treatment for increasing levels of maturation upon further differentiation. FIG. 7B illustrates the P1S5D+none treatment analyzed in FIGS. 7B and 7C for increasing levels of maturation upon further differentiation. FIG. 7B also depicts graphs showing analyses of the P1S5D+none treatment through day 37. FIG. 7C depicts graphs showing the time course quantitative analyses of electrophysiological properties of P1S5D+none conditions through day 37. As time proceeded, resting membrane potential became hyperpolarized, input resistance decreased, Na+−channel current increased, action potential threshold decreased and the maximum firing frequency increased. Statistics was carried out first using ordinary one-way ANOVA to determine if statistically significant differences exist among the means of each group: F=0.3222, P=0.8093, R²=0.0248 (REM); F=7.554, P=0.0023, R²=0.5862 (half-width); F=0.7654, P=0.5209, R²=0.0560 (rising Tau); F=4.88, P=0.006, R²=0.2891 (input resistance); F=7.364, P=0.0005, R²=0.3676 (frequency). Then the Dunnett's multiple comparison test was used to compare mean values of each group to day 16. Only those comparisons that are significant were marked on the graph. Error bars represent s. e. m. * P<0.05, ** P<0.01, *** P<0.001.

FIGS. 8A-8G depict illustrations, images and graphs regarding the co-culture of hPSC-derived neurons with astrocyte or astrocyte conditioned medium. FIG. 8A is a schematic illustration of long-term maintenance of P8S10D+D neurons with astrocyte co-culture or conditioned media. FIG. 8B depicts bright field images of P8S10D+D neurons co-cultured with astrocytes or conditioned media at day 25 and day 35. FIG. 8C depicts graphs showing representative traces of action potential firings of P8S10D+D neurons cocultured with astrocytes or conditioned media at day 25 and day 35, evoked by somatic current injection from −30 to +100 pA. FIG. 8D depicts graphs showing quantitative analyses of passive membrane properties and action potential properties. FIG. 8E depicts images of MAP2ab staining of P8S10D+D neurons with astrocytes co-culture, which show increased complexity of dendrite branching with time in culture. FIG. 8F depicts a graph showing a sholl analysis at day 36 of P8S10D+D neurons co-cultured with astrocytes, compared with neurons cultured with conditioned medium alone. Scale bars: 50 m. Error bars represent s. e. m. *** P<0.001. FIG. 8G depicts bright field images of P8S10D+D neurons co-cultured with astrocytes at day 90. N=15, 23, 7, 5 cells recorded for cultures with astrocytes at day 25, 35, and cultures in conditioned medium at day 25, 35.

FIG. 9 depicts a schematic illustration of PSD treated cells engraftment into a neonatal mouse.

FIGS. 10A-10E depict images showing extensive axonal projections and integration of hPSC-derived neuron using P1S5D induction grafted into the neonatal mouse brain as assessed by iDISCO18-based whole mount brain imaging. FIG. 10A depicts a dorsal view of the graft core and its cortical projections at 1.5 months. FIG. 10B depicts analysis of the grafted brain at 1-6 months post-grafting using whole brain immunohistochemistry and imaging by light-sheet microscopy. Dorsal view of the graft core and its cortical projections at 1.5 months. FIG. 10C depicts projections (100 μm thick) showing the graft core morphology and the major projection regions (frontal cortex and corpus callosum). Dotted line: midline, arrow: aberrant longitudinal projections in the corpus callosum. FIG. 10D depicts iDISCO based imaging of half brains showing the morphology of the graft projections at 1.5, 3 and 6 months. Top panels: views of the half brain showing the graft cores and their major cortical projections. Central panels: projections (100 μm thick) showing the details of the fiber morphology from the boxed regions, and their increased branching over time. Lower panel: projections (100 μm thick) showing hSynaptophysin co-labeled with GFP in the hippocampus. The hSyn signal was absent at 1.5 months, extremely faint at 3 months, but very high at 6 months. FIG. 10E depicts immunohistochemical analysis for markers of cortical identity in graft derived neurons identified by human specific cytoplasm marker (SC121) or GFP expression. ˜60% of the grafted cells expressed TBR1, ˜50% expressed CTIP2 (more than half of all CTIP2+ cells co-expressed TBR1), and ˜30% expressed SATB2. N=5 animals for 1, 1.5 and 3 months analyzed, and 2 animals for 6 months analyzed. Scale bars: 500 μm (b, c, d, top and middle panels), 50 μm (d, bottom panel and e).

FIGS. 11A-11B depict images of iDISCO based whole brain immunofluorescence analyses of P8S10D and LSB+XAV grafts at 1 month after transplantation. FIG. 11A depicts images of a P8S10D grafted half brain, stained for GFP (whole view and details of the frontal cortical region). P8S10D grafts showed inconsistent survival after transplantation into neonatal mouse cortex. However, animals with surviving graft showed long fiber projections across cortical regions. GFP+ cells devoid of axons were abundantly detected outside of the graft (boxed region). FIG. 11B depicts images of a LSB+X grafted half brain, stained for GFP. Whole view (side and dorsal) and detail of the graft margin (boxed region). LSB+X grafts showed massive overgrowth in host brain resulting in tumor-like structures with very limited evidence of neuronal differentiation and maturation. Only a few short fiber tracts can be seen at the margin of the graft (boxed region). N=2 animals for P8S10D condition, and 4 animals for LSB+XAV condition analyzed. Scale bars: 500 μm.

FIGS. 12A-12D depict images of trajectories and morphologies of P1S5D grafted neurons at 1.5 months after transplantation. FIG. 12A depicts images showing that landmarks of the adult mouse brain can be revealed by tissue autofluorescence. 100 m thick maximum projection of optical sections taken at the center of an adult mouse brain after iDISCO processing showing the major myelinated tracts from 488 nm laser excitation of the endogenous fluorescence. FIG. 12B depicts images showing examples of axons from grafted neurons not following major pathways. Maximum projections, 100 m thick, of whole iDISCO treated 1.5 months old mouse brains grafted at birth, stained for GFP. In the striatum, GFP+ axons descending from the grafted neurons in the cortex are seen mainly outside of the main descending tracts. In the cortex, large bundles of GFP+ axons are observed in aberrant position. FIG. 12C depicts images showing examples of axons from grafted neurons following major pathways. iDISCO treated 1.5 months old mouse brain grafted at birth, stained for GFP. In the hippocampus, grafted neurons present in CA3 (arrow) are sending axons along the fimbria tract towards the septum. In the cortex, grafted neurons cross hemisphere following callosal axons. FIG. 12D depicts images of a few morphologies seen in the grafted brains: most neurons are compacted in the graft core, and therefore their dendritic morphologies are masked. However, a few GFP+ neurons are found outside of the graft core and exhibit diverse morphologies. N=3, Scale bars are 1 mm (a), 500 m (b,c) and 200 m (d).

FIGS. 13A-13D depict images and diagrams showing the electrophysiology of P1S5D grafted neurons in vivo. FIGS. 13A and 13B show recordings of action potentials and firing patterns, as well as sEPSCs from a GFP+ graft at P10 (FIG. 13A) and P30 (FIG. 13B), respectively, with unusually mature properties. FIGS. 13C and 13D show that most GFP+ cells in grafts exhibited more immature firing patterns as illustrated by representative action potentials in FIG. 13C and sEPSCs in FIG. 13D recorded at P15 and P45. N=4 animals for P1S5D condition analyzed, and 1 animal for P8S10 condition analyzed.

FIG. 14 is a summary of the rapid cortical neuron induction paradigm. Dual SMAD inhibition by LSB inhibits trophectoderm, mesendoderm, and non-neural ectoderm cell fates promoting CNS fates. XAV939 promotes anterior CNS identity while SU5402/PD0325901 accelerate exit from pluripotency toward neuroectodermal fates. A highly transient anterior neuroectodermal precursor state is driven toward post-mitotic cortical fates in the presence of DAPT and SU5402/PD0325901. Immature cortical neurons can acquire functional maturity in vitro by day 16 of differentiation and day 8 neurons, grafted into neonatal mouse host brain, show widespread axonal projections and integration in cortex.

FIG. 15A-15G shows a dosage-dependent response of proliferation and viability upon P/S/D treatment. (a) Percentage of mitotic cells expressing phospho-histone 3 (pH3) among total cells at one day after P/S/D treatment (day 3 of differentiation). (b) Percentage of apoptotic cells expressing cleaved caspase 3 (CC3) among total cells at one day after P/S/D treatment (day 3 of differentiation). The conditions highlighted in the dashed line boxes in a,b are the P1 dosage groups aligned by ascending order of S concentration. For a,b, N=4 randomly selected photo frames from each of the 2 independent batches of cell cultures. Statistical analysis was carried out using the Dunnett's multiple comparison test to compare each dosage with LSB+X at day 3 of differentiation. Only those comparisons that are significantly different from LSB+X are marked on the graph. (c) Summary of grouped results in a following the order of increasing concentration of P, or increasing concentration of S (d). (e) Summary of grouped results in b following the order of increasing concentration of P, or increasing concentration of S (f). For c-f, statistical analysis was carried out using the Dunnett's multiple comparison test to compare each dosage of P with the no P group (c,e) and each dosage of S with the no S dosage groups (d,f). (g) Nomenclature for the various dosage groups shown in c-f. Black dots represent values from quantification of individual photos frames. Error bars represent s. e. m. * P<0.05, ** P<0.01, *** P<0.001, **** P<0.0001.

FIG. 16A-16D shows characterization of additional fate markers at day 13 of differentiation. (a) Expression of layer VI marker TLE4 in post-mitotic neurons, and CUX2 expression in progenitors at day 13 of differentiation in both P1S5D and P8S10D induction. (b) Quantification of mRNA expression at day 13 of markers other than TBR1 representing different brain areas and fates. N=3 independent batches of cell cultures. Cortical progenitor marker OTX2, ZNF521, BRN2 and COUPTF1, layer V cortical neuron marker CTIP2, and the vesicular glutamate transporter VGLUT1 are upregulated compared to LSB+X. (c) Expression of BRN3A+ and ISL1+ cells in both P1S5D and P8S10D cultures at day 13. (d) Quantification of BRN3A+ cells at day 13. Scale bars: 50 μm. Error bars represent s. e. m.

FIG. 17 shows molecular characterization of long-term culture beyond day 13. Quantification of mRNAs expression in P1S5D and P8S10D treated culture (FIG. 3F) compared to LSB+X treated cultures. RGS4: cortical layer II-III,V marker. CHX10: retinal marker. GFAP, AQP4: astrocyte marker. OLIG2: oligodendrocyte precursor marker. For long-term culture of LSB+X cells, cells were maintained in N2 medium without re-adding small-molecule inhibitors. N=3 independent batches of cell cultures. Error bars represent s. e. m.

FIG. 18A-18G shows Generation of the CUX2-CreER^{T2} conditional reporter hPSC line and early generation of CUX2+ neurons in both P1S5D and P8S10D treated cells. (a) Design of the homology donor targeting the CUX2 first exon. The selection cassette was excised upon expression of Flp recombinase after transgenesis. The location of primer sequences to confirm targeting is shown. (b) Schematic illustration of the targeted alleles. The transgenic lines express CreER^{T2} from the CUX2 locus and the FLEX-tdTomato conditional reporter under the CAG promoter at the AAVS1 safe harbor locus. Expression of CUX2 in the presence of 4OHT induces recombination at the reporter locus and tdTomato expression. (c) PCR confirmation of targeted transgenesis. Lanes 1-2 confirm 5′ and 3′ CreER^{T2} insertions at the CUX2 genomic locus, respectively, while lanes 3-4 confirm 5′ and 3′ CAG-FLEX/tdTomato insertions at the AAVS1 genomic locus, respectively. (d) Genomic DNA sequencing at the CUX2 locus confirms successful targeting of one allele (SEQ ID NO: 5) and a wild-type non-targeted allele (SEQ ID NO: 6). (e) tdTomato positive post-mitotic neurons at day 70 in culture after 4OHT induction (i). Typical pyramidal morphology and lengthy projections can be observed upon higher magnification (ii). (f) CUX2+ neurons with mature morphologies observed in P1S5D and P8S10D culture at day 33 of differentiation. (g) Pyramidal morphology at day 33 suggests cortical projection neuron identify of P1S5D and P8S10D neurons. Scale bars in e (i) represents 100 μm, while those in others represent 50 μm.

FIG. 19 shows a checklist for characterizing cells at day 13 of P1S5D and P8S10D differentiation (other than TBR1+ neurons).

FIG. 20 shows primers used for CUX2 reporter line genotyping (SEQ ID NOS 7-16, respectively, in order of appearance).

FIG. 21 shows a summary of daily culture protocols for P1S5D and P8S10D cells.

### 5. DETAILED DESCRIPTION

The presently disclosed subject matter relates to methods of preparing cortical neurons derived from human stem cells, e.g. by in vitro differentiation of human stem cells to functional cortical neurons, and cells produced by such methods. Also provided are uses of such cells for treating a CNS neurodegenerative disorder.

For purposes of clarity of disclosure and not by way of limitation, the detailed description is divided into the following subsections:
5.1 Definitions;
5.2 Methods of Differentiating Stem Cells;
5.3 Compositions Comprising Differentiated Cell Populations;
5.4 Methods of Preventing and/or Treating CNS Neurodegenerative Disorders;
and
5.5 Kits

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, a wingless (Wnt) complex protein, including beta-catnin, NOTCH, transforming growth factor beta (TGFβ), Activin, Nodal and glycogen synthase kinase 3β (GSK3P) proteins, FGF and MAPK/ERK (MEK) proteins. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TFGβ), Activin, Nodal, bone morphogenic proteins (BMPs), etc.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, a named associated molecule, such as a glycogen synthase kinase 3β (GSK3β)) (e.g., including, but not limited to, the signaling molecules described herein). For one example, an inhibitor of SMAD signaling can function, for example, via directly contacting SMAD, contacting SMAD mRNA, causing conformational changes of SMAD, decreasing SMAD protein levels, or interfering with SMAD interactions with signaling partners, and affecting the expression of SMAD target genes. Inhibitors also include molecules that indirectly regulate SMAD biological activity by intercepting upstream signaling molecules (e.g., within the extracellular domain). Examples of a SMAD signaling inhibitor molecules and an effect include: Noggin which sequesters bone morphogenic proteins, inhibiting activation of ALK receptors 1,2,3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules. Antibodies that block activins, nodal, TGFβ, and BMPs are contemplated for use to neutralize extracellular activators of SMAD signaling, and the like. Although the foregoing example relates to SMAD signaling inhibition, similar or analogous mechanisms can be used to inhibit other signaling molecules. Examples of inhibitors include, but are not limited to: LDN193189 (LDN) and SB431542 (SB) (LSB) for SMAD signaling inhibition, XAV939 (X) for Wnt inhibition, SU5402 (S) for FGF signaling inhibition, DAPT (D) for Notch signaling inhibition, and PD0325901 (P) for MAPK/ERK (MEK) signaling inhibition.

Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators”, as used herein, refer to compounds that increase, induce, stimulate, activate, facilitate, or enhance activation the signaling function of the molecule or pathway, e.g., Wnt signaling.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 5,000 cells or at least about 10,000 cells or at least about 100,000 cells or at least about 1,000,000 cells. The population may be a pure population comprising one cell type, such as a population of cortical neuron precursors, or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells. A human stem cell refers to a stem cell that is from a human.

As used herein, the term “embryonic stem cell” refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years. For example, “embryonic stem cell” can refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self renewal (in the laboratory) and differentiation. Such cells vary in their differentiation capacity, but it is usually limited to cell types in the organ of origin.

As used herein, the term “neuron” refers to a nerve cell, the principal functional units of the nervous system. A neuron consists of a cell body and its processes—an axon and one or more dendrites. Neurons transmit information to other neurons or cells by releasing neurotransmitters at synapses.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as enteric neuron precursors.

As used herein, the term “directed differentiation” in reference to a stem cell refers to the use of small molecules, growth factor proteins, and other growth conditions to promote the transition of a stem cell from the pluripotent state into a more mature or specialized cell fate (e.g. cortical neurons, etc.).

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein, such as TUJI, DCX, TBR1, REELIN, and FOXG1).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” cells with a compound (e.g., one or more inhibitor, activator, and/or inducer) refers to exposing cells to a compound, for example, placing the compound in a location that will allow it to touch the cell. The contacting may be accomplished using any suitable methods. For example, contacting can be accomplished by adding the compound to a tube of cells. Contacting may also be accomplished by adding the compound to a culture medium comprising the cells. Each of the compounds (e.g., the inhibitors, activators, and molecules that induce vagal neural crest patterning disclosed herein) can be added to a culture medium comprising the cells as a solution (e.g., a concentrated solution). Alternatively or additionally, the compounds (e.g., the inhibitors, activators, and molecules that induce vagal neural crest patterning disclosed herein) as well as the cells can be present in a formulated cell culture medium.

An effective amount is an amount that produces a desired effect.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

### 5.2 Methods of Differentiation Stem Cells

The presently disclosed subject matter provides for in vitro methods for inducing differentiation of stem cells (e.g., human stem cells). Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In certain embodiments, the human stem cell is a human induced pluripotent stem cell (hiPSC). In certain embodiments, the stem cells are non-human stem cells. Non-limiting examples of non-human stem cells non-human primate stem cells, rodent stem cells, dog stem cells, cat stem cells. In certain embodiments, the stem cells are pluripotent stem cells. In certain embodiments, the stem cells are embryonic stem cells. In certain embodiments, the stem cells are induced pluripotent stem cells.

The present invention discloses methods of differentiating stem cells into cortical neurons, or precursors thereof. Without being limited to any theory, the present invention discloses that contacting a stem cell with an inhibitor of MAPK/ERK kinase accelerates the differentiation of cortical neurons from stem cells that are contacted with one or more inhibitor of transforming growth factor beta (TGFβ)/Activin-Nodal signaling; one or more inhibitor of bone morphogenetic protein (BMP) signaling; one or more inhibitor of Wnt signaling; one or more inhibitor of FGF signaling; and one or more inhibitor of Notch signaling.

In certain embodiments, “Wnt” or “wingless” in reference to a ligand refers to a group of secreted proteins (e.g. Intl (integration 1) in humans) capable of interacting with a Wnt receptor, such as a receptor in the Frizzled and LRPDerailed/RYK receptor family.

In certain embodiments, the term “Wnt” or “wingless” in reference to a signaling pathway refers to a signal pathway composed of Wnt family ligands and Wnt family receptors, such as Frizzled and LRPDerailed/RYK receptors, mediated with or without β-catenin. In certain embodiments, a Wnt signaling pathway includes mediation by β-catenin, e.g., WNT 4/β-catenin.

In certain embodiments, a presently disclosed differentiation method comprises contacting a population of stem cells with one or more inhibitor of transforming growth factor beta (TGFβ)/Activin-Nodal signaling, which thereby inhibits Small Mothers Against Decapentaplegic (SMAD) signaling. In certain embodiments, the inhibitor of TGFβ/Activin-Nodal signaling neutralizes the ligands including TGFβs, bone morphogenetic proteins (BMPs), Nodal, and activins, or blocking their signal pathways through blocking the receptors and downstream effectors. Non-limiting examples of inhibitors of TGFβ/Activin-Nodal signaling are disclosed in WO/2010/096496, WO/2011/149762, WO/2013/067362, WO/2014/176606, WO/2015/077648, Chambers et al., Nature Biotechnology 27, 275-280 (2009), and Chambers et al., Nature biotechnology 30, 715-720 (2012), which are incorporated by reference in their entireties for all purposes. In certain embodiments, the one or more inhibitor of TGFβ/Activin-Nodal signaling is a small molecule selected from the group consisting of SB431542, derivatives thereof, and mixtures thereof. “SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

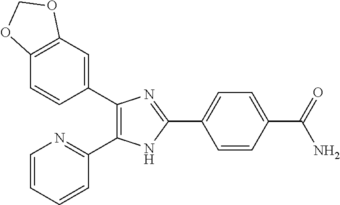 [SMILES: NC(=O)c1ccc(-c2nc(-c3ccc4c(c3)OCO4)c(-c3ccccn3)[nH]2)cc1]

The presently disclosed differentiation method further comprises contacting the stem cells with one or more inhibitor of BMP signaling, which thereby inhibits Small Mothers Against Decapentaplegic (SMAD) signaling. Non-limiting examples of inhibitors of BMP signaling are disclosed in WO/2010/096496, WO/2011/149762, WO/2013/067362, WO/2014/176606, WO/2015/077648, Chambers et al., Nature Biotechnology 27, 275-280 (2009), and Chambers et al., Nature biotechnology 30, 715-720 (2012), which are incorporated by reference in their entireties for all purposes. In certain embodiments, the one or more inhibitor of SMAD signaling is a small molecule selected from the group consisting of LDN193189, derivatives thereof, and mixtures thereof. “LDN193189” refers to a small molecule DM-3189, IUPAC name 4-(6-(4-(piperazin-1-yl)phenyl)pyrazolo[1,5-a]pyrimidin-3-yl)quinoline, with a chemical formula of C₂₅H₂₂N₆. LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18: 4388-4392, herein incorporated by reference). In certain embodiments, LDN193189 has the following structure:

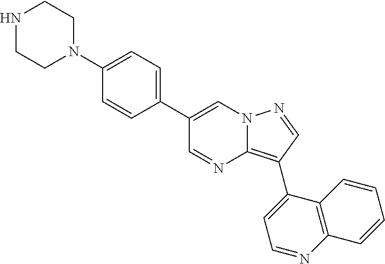 [SMILES: c1ccc2c(-c3cnn4cc(-c5ccc(N6CCNCC6)cc5)cnc34)ccnc2c1]

The present invention further provides for methods of differentiating a population of stem cells into cortical neurons or precursors thereof, wherein the cells are contacted with one or more inhibitor of Wnt signaling, for example, but not limited to XAV399, a tankyrase inhibitor (Huang et al. Nature 461, 614-620 (2009)), Dickkopf (Dkk) proteins, secreted Frizzled-Related Proteins (sFRPs), IWR (Chen et al. Nature Chemical Biology 5(2): 100-107 (2009); Kulak et al., Molecular and Cellular Biology, 4; 35(14):2425-35 (2015)), 2,4-diamino-quinazoline (Chen et al., Bioorganic & medicinal chemistry letters, 1; 19(17):4980-3 (2009)), IWP (Chen et al., Nat Chem Biol. 2009 February; 5(2):100-7), LGK974, C59 (Proffitt et al., Cancer Res. 2013 Jan. 15; 73(2):502-7), Ant 1.4Br/Ant 1.4Cl (Morrell et al., PLoS One. 2008 Aug. 13; 3(8):e2930), Niclosamide (Chen et al., Biochemistry. 2009 Nov. 3; 48(43):10267-74), apicularen and bafilomycin (Cruciat et al., Science. 2010 Jan. 22; 327(5964):459-63), G007-LK and G244-LM (Lau et al., Cancer Res. 2013 May 15; 73(10):3132-44), pyrvinium (Thorne et al., Nat Chem Biol. 2010 November; 6(11):829-36), NSC668036 (Shan et al., Biochemistry. 2005 Nov. 29; 44(47):15495-503), Quercetin (Park et al., Biochem Biophys Res Commun. 2005 Mar. 4; 328(1):227-34), ICG-001 (Emami et al., Proc Natl Acad Sci USA. 2004 Aug. 24; 101(34):12682-7), PKF115-584 (Lepourcelet et al., Cancer Cell. 2004 January; 5(1):91-102), BC2059 (Fiskus et al., Leukemia. 2015 June; 29(6):1267-78), Shizokaol D (Tang et al., PLoS One. 2016 Mar. 24; 11(3):e0152012), and derivatives thereof.

In certain embodiments, XAV399 is 3,5,7,8-Tetrahydro-2-[4-(trifluoromethyl)phenyl]-4H-thiopyrano[4,3-d]pyrimidin-4-one, having the chemical formula C₁₄H₁₁F₃N₂OS. In certain embodiments, XAV399 has the following structure:

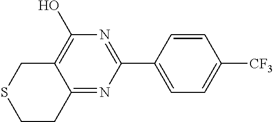 [SMILES: Oc1nc(-c2ccc(C(F)(F)F)cc2)nc2c1CSCC2]

In certain embodiments, the population of stem cells are contacted with an effective amount of one or more inhibitor of TGFβ/Activin-Nodal signaling, an effective amount of one or more inhibitor of BMP signaling, and an effective amount of one or more inhibitor of Wnt signaling, wherein the cells are contacted to the inhibitors for at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 or more days, wherein the cells are contacted with a concentration of said compounds effective to produce a population of cells expressing one or more markers of cortical neurons or precursors thereof.

In certain embodiments, the cells are contacted with effective amounts of the inhibitors for up to about 4, up to about 5, up to about 6, up to about 7, up to about 8, up to about 9, up to about 10, up to about 11, up to about 12, up to about 13, up to about 14, up to about 15, up to about 16, up to about 17, up to about 18, up to about 19, up to about 20 or more days, wherein the cells are contacted with a concentration of said compounds effective to produce a population of cells expressing one or more markers of cortical neurons or precursors thereof.

In certain embodiments, the cells are contacted with effective amounts of the inhibitors for about 4, 5, 6, 7, 8, 9, 10, 11, 12, 13, 14, 15, 16, 17, 18, 19, 20 or more days, wherein the cells are contacted with a concentration of said compounds effective to produce a population of cells expressing one or more markers of cortical neurons or precursors thereof.

In certain embodiments, the day whereby the cells are contacted with the one or more inhibitor of TGFβ/Activin-Nodal signaling corresponds to day 0, and the cells are contacted to the inhibitors for between days 0 and 6, or between day 0 and day 7.

In certain embodiments, the cells are contacted with an inhibitor of transforming growth factor beta (TGFβ)/Activin-Nodal signaling at a concentration of between about 1 and 20 μM, between about 2 and 18 μM, between about 4 and 16 μM, between about 6 and 14 μM, between about 8 and 12 μM, or about 10 μM.

In certain embodiments, the cells are contacted with an inhibitor of transforming growth factor beta (TGFβ)/Activin-Nodal signaling at a concentration of between about 1 and 18 μM, between about 1 and 16 μM, between about 1 and 14 μM, between about 1 and 12 μM, between about 1 and 10 μM, between about 1 and 8 μM, between about 1 and 6 μM. between about 1 and 4 μM, or between about 1 and 2 μM.

In certain embodiments, the cells are contacted with an inhibitor of transforming growth factor beta (TGFβ)/Activin-Nodal signaling at a concentration of between about 2 and 20 μM, between about 4 and 20 μM, between about 6 and 20 μM, between about 8 and 20 μM, between about 10 and 20 μM, between about 12 and 20 μM, between about 14 and 20 μM, between about 16 and 20 μM, or between about 18 and 20 μM.

In certain embodiments, the cells are contacted with an inhibitor of BMP signaling at a concentration of between about 10 and 500 nM, between about 25 and 475 nM, between about 50 and 450 nM, between about 100 and 400 nM, between about 150 and 350 nM, between about 200 and 300 nM, or about 250 nM or about 100 nM, or about 50 nM.

In certain embodiments, the cells are contacted with an inhibitor of BMP signaling at a concentration of between about 10 and 475 nM, between about 10 and 450 nM, between about 10 and 400 nM, between about 10 and 350 nM, between about 10 and 300 nM, between about 10 and 250 nM, between about 10 and 200 nM, between about 10 and 150 nM, between about 10 and 100 nM, or between about 10 and 50 nM.

In certain embodiments, the cells are contacted with an inhibitor of BMP signaling at a concentration of between about 25 and 500 nM, between about 50 and 500 nM, between about 100 and 500 nM, between about 150 and 500 nM, between about 200 and 500 nM, between about 250 and 500 nM, between about 300 and 500 nM, between about 350 and 500 nM, between about 400 and 500 nM, or between about 450 and 500 nM.

In certain embodiments, the cells are contacted with an inhibitor of Wnt signaling at a concentration of between about 0.1 and 10 μM, between about 0.5 and 8 μM, between about 1 and 6 μM, between about 2 and 5.5 μM, or about 5 μM, or about 2 μM. or about 1 μM.

In certain embodiments, the cells are contacted with an inhibitor of Wnt signaling at a concentration of between about 0.1 and 8 μM, between about 0.1 and 6 μM, between about 0.1 and 4 μM, between about 0.1 and 2 μM, between about 0.1 and 1 μM, or between about 0.1 and 0.5 μM.

In certain embodiments, the cells are contacted with an inhibitor of Wnt signaling at a concentration of between about 0.5 and 10 μM, between about 1 and 10 μM, between about 2 and 0 μM, between about 4 and 10 μM, between about 6 and 10 μM, or between about 8 and 10 μM.

In certain embodiments, the stem cells are further contacted with an effective amount of one or more inhibitor of FGF signaling, for example, SU5402 (Sun et al., Journal of medicinal chemistry 42, 5120-5130 (1999); Paterson et al. Br. J. Haematol. 124 595 (2004); Tanaka et al., Nature 435:172 (2005)), PD 173074 (N-[2-[[4-(Diethylamino)butyl]amino]-6-(3,5-dimethoxyphenyl)pyrido[2,3-d]pyrimidin-7-yl]-N′-(1,1-dimethylethyl)urea; Bansal et al., J. Neurosci. Res., 2003; 74:486), FIN 1 hydrochloride (N-(3-((3-(2,6-dichloro-3,5-dimethoxyphenyl)-7-(4-(diethylamino)butylamino)-2-oxo-3,4-dihydropyrimido[4,5-d]pyrimidin-1(2H)-yl)methyl)phenyl)acrylamide; Zhou, Chem. Biol., 2010; 17:285), SU6668 (5-[1,2-Dihydro-2-oxo-3H-indol-3-ylidene)methyl]-2,4-dimethyl-1H-pyrrole-3-propanoic acid; Yamamoto et al., Cancer Res. 2008 Dec. 1; 68(23):9754-62), PD 166285 dihydrochloride (6-(2,6-Dichlorophenyl)-2-[[4-[2-(diethylamino)ethoxy]phenyl]amino]-8-methylpyrido[2,3-d]pyrimidin-7(8H)-one dihydrochloride; Panek et al., J Pharmacol Exp Ther. 1997 December; 283(3):1433-44), PD 161570 (N-[6-(2,6-Dichlorophenyl)-2-[[4-(diethylamino)butyl]amino]pyrido[2,3-d]pyrimidin-7-yl]-N′-(1,1-dimethylethyl)urea; Hamby et al., J Med Chem. 1997 Jul. 18; 40(15):2296-303), AP 24534 (3-(2-Imidazo[1,2-b]pyridazin-3-ylethynyl)-4-methyl-N-[4-[(4-methyl-1-piperazinyl)methyl]-3-(trifluoromethyl)phenyl]-benzamide; Huang et al., J. Med. Chem., 2010; 53:4701), or derivatives thereof.

In certain embodiments, the term “SU5402” refers to a small molecule with a chemical formula of C₁₇H₁₆N₂O₃ and chemical name: 2-[(1,2-Dihydro-2-oxo-3H-indol-3-ylidene)methyl]-4-methyl-1H-pyrrole-3-pr-opanoic acid. In certain embodiments, SU5402 has the following structure:

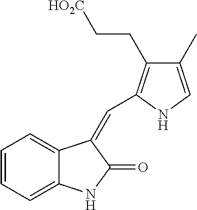 [SMILES: Cc1c[nH]c(/C=C2\C(=O)Nc3ccccc32)c1CCC(=O)O]

In certain embodiments, the stem cells are further contacted with an effective amount of one or more inhibitor of Notch signaling, for example, DAPT (Dovey et al., Journal of neurochemistry 76, 173-181 (2001)), Begacestat (5-Chloro-N-[(1S)-3,3,3-trifluoro-1-(hydroxymethyl)-2-(trifluoromethyl)propyl]-2-thiophenesulfonamide; Mayer et al., J. Med. Chem. 51:7348 (2008)), DBZ (N-[(1S)-2-[[(7S)-6,7-Dihydro-5-methyl-6-oxo-5H-dibenz[b,d]azepin-7-yl]amino]-1-methyl-2-oxoethyl]-3,5-difluorobenzeneacetamide; van Es et al., Nature 435:959 (2005)), BMS 299897 (2-[(1R)-1-[[(4-Chlorophenyl)sulfonyl](2,5-difluorophenyl)amino]ethyl-5-fluorobenzenebutanoic acid; Goldstein et al., J. Pharmacol. Exp. Ther. 323:102 (2007)), Compound W (3,5-Bis(4-nitrophenoxy)benzoic acid; Okochi et al., J. Biol. Chem. 281:7890 (2006)), Flurizan ((R)-2-Fluoro-α-methyl[1,1′-biphenyl]-4-acetic acid; Eriksen et al., J. Clin. Invest. 112:440 (2003)), L-685,458 ((5S)-(tert-Butoxycarbonylamino)-6-phenyl-(4R)-hydroxy-(2R)-benzylhexanoyl)-L-leucy-L-phenylalaninamide; Shearman et al., Biochemistry 39:8698 (2000)), JLK 6 (7-Amino-4-chloro-3-methoxy-1H-2-benzopyran; Petit et al., Nat. Cell. Biol. 3:507 (2001)), MRK 560 (N-[cis-4-[(4-Chlorophenyl)sulfonyl]-4-(2,5-difluorophenyl)cyclohexyl]-1,1,1-trifluoromethanesulfonamide; Best et al., J. Pharm. Exp. Ther. 317:786 (2006)), PF 3084014 hydrobromide ((2S)-2-[[(2S)-6,8-Difluoro-1,2,3,4-tetrahydro-2-naphthalenyl]amino]-N-[1-[2-[(2,2-dimethylpropyl)amino]-1,1-dimethylethyl]-1H-imidazol-4-yl]pentanamide dihydrobromide; Lanz et al., J. Pharmacol. Exp. Ther. 334:269 (2010)), or derivatives thereof.

In certain embodiments, the term “DAPT” refers to one example of a γ-secretase inhibitor that inhibits NOTCH which is described as a dipeptidic γ-secretase-specific inhibitor otherwise known as N-[(3,5-Difluorophenyl)acetyl]-L-alanyl-2-phenyl]glycine-1,1-dimethyl ethyl ester; LY-374973, N—[N-(3,5-Difluorophenacetyl)-L-alanyl]-S-phenylglycine t-butyl ester; N—[N-(3,5-difluorophenacetyl)-L-alanyl]-S-phenylglycine t-butyl ester; with a chemical formula of C₂₃H₂₆F2N₂O₄. One example of a DAPT derivative is DAP-BpB (N—[N-(3,5-difluorophenacetyl)-L-alanyl]-(S)-phenylglycine-4-(4-(8-bioti-namido)octylamino)benzoyl)benzyl)methylamide), a photoactivable DAPT derivative. In certain embodiments, DAPT has the following structure:

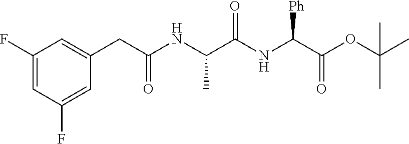 [SMILES: C[C@H](NC(=O)Cc1cc(F)cc(F)c1)C(=O)N[C@H](C(=O)OC(C)(C)C)c1ccccc1]

In certain embodiments, the stem cells are further contacted with an effective amount of one or more inhibitor of MAPK/ERK kinase, for example, PD198306 (Ciruela et al., British Journal of Pharmacology 138(5):751-6 (2003); Pelletier et al., Arthritis & Rheumatism, 48: 1582-1593 (2003)), PD0325901 (N-[(2R)-2,3-Dihydroxypropoxy]-3,4-difluoro-2-[(2-fluoro-4-iodophenyl)amino]-benzamide; Barrett et al., Bioorganic & medicinal chemistry letters 18, 6501-6504 (2008)), 10Z-Hymenialdisine ((4Z)-4-(2-Amino-1,5-dihydro-5-oxo-4H-imidazol-4-ylidene)-2-bromo-4,5,6,7-tetrahydropyrrolo[2,3-c]azepin-8(1H)-one; Breton et al., J. Pharmacol. Exp. Ther. 282:459(1997)), PD 184352 (2-[(2-Chloro-4-iodophenyl)amino]-N-cyclopropylmethoxy)-3,4-difluorobenzamide; Allen et al., Semin. Oncol. 30:105 (2003)), PD 198306 (N-(Cyclopropylmethoxy)-3,4,5-trifluoro-2-[(4-iodo-2-methylphenyl)amino]-benzamide; Pelletier et al., Arthrit. Rheumat. 48:1582(2003)), PD 334581 (N-[5-[3,4-Difluoro-2-[(2-fluoro-4-iodophenyl)amino]phenyl]-1,3,4-oxadiazol-2-yl]-4-morpholineethanamine; Ohren et al., Nat. Struct. Mol. Biol. 11:1192(2004)), PD 98059 (2-(2-Amino-3-methoxyphenyl)-4H-1-benzopyran-4-one; Dudley et al., Proc. Natl. Acad. Sci. U.S.A. 92:7686 (1995)), SL 327 (α-[Amino[(4-aminophenyl)thio]methylene]-2-(trifluoromethyl)benzeneacetonitrile; Wang et al., J. Pharmacol. Exp. Ther. 304:172(2003)), U1024 (Bis[amino(methylthio)methylene]butanedinitrile; Favata et al., J. Biol. Chem. 273:18623(1998)), U0126 (1,4-Diamino-2,3-dicyano-1,4-bis[2-aminophenylthio]butadiene; Favata et al., J. Biol. Chem. 273:18623(1998)), Arctigenin ((3R,4R)-4-[(3,4-Dimethoxyphenyl)methyl]dihydro-3-[(4-hydroxy-3-methoxyphenyl)methyl]-2(3H)-furanone; Jang et al. J. Neurosci. Res. 68:233(2002)), BIX 02189 ((3Z)-3-[[[3-[(Dimethylamino)methyl]phenyl]amino]phenylmethylene]-2,3-dihydro-N,N-dimethyl-2-oxo-1H-indole-6-carboxamide; Tatake et al., Biochem. Biophys. Res. Comm. 377:120 (2008)), or derivatives thereof.

In certain embodiments, the term “PD0325901” refers to a small molecule with a chemical formula of C₁₆H₁₄F₃IN₂O₄ and chemical name N-(2,3-dihydroxy-propoxy)-3,4-difluoro-2-(2-fluoro-4-iodo-phenylamino)-benzamide. In certain embodiments, PD0325901 has the following structure:

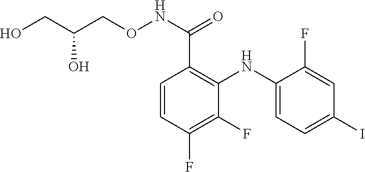 [SMILES: O=C(NOC[C@H](O)CO)c1ccc(F)c(F)c1Nc1ccc(I)cc1F]

In certain embodiments, the effective amounts of inhibitors are contacted to the cells for at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 or more days.

In certain embodiments, the cells are contacted to the effective amounts of one or more inhibitor of FGF signaling, one or more inhibitor of Notch signaling, and one or more inhibitor of MAPK/ERK kinase for up to 4, up to 5, up to 6, up to 7, up to 8, up to 9, up to 10, up to 11, up to 12, up to 13, up to 14, up to 15, up to 16, up to 17, up to 18, up to 19, up to 20 or more days.

In certain embodiments, the effective amounts of one or more inhibitor of FGF signaling, one or more inhibitor of Notch signaling, and one or more inhibitor of MAPK/ERK kinase are contacted to the human stem cells at least about 1, at least about 2, at least about 3, at least about 4 or at least about 5 days after the cells are initially contacted with effective amounts of the one or more inhibitor of TGFβ/Activin-Nodal signaling, one or more inhibitor of BMP signaling, and/or one or more inhibitor of Wnt signaling. In certain embodiments, the cells are initially contacted with an effective amount(s) of the (iv), (v) and/or (vi) inhibitor about 24 hours, about 48 hours, about 72 hours, about 96 hours, about 120 hours, or about 144 hours after the cells are initially contacted with effective amounts of (i), (ii) and (iii) inhibitors.

In certain embodiments, effective amounts of the one or more inhibitor of FGF signaling, one or more inhibitor of Notch signaling, and one or more inhibitor of MAPK/ERK kinase are contacted to the human stem cells up to about 1, 2, 3, 4 or 5 days after the cells are initially contacted with effective amounts of the one or more inhibitor of TGFβ/Activin-Nodal signaling, one or more inhibitor of BMP signaling, and/or one or more inhibitor of Wnt signaling.

In certain embodiments, the cells are contacted to the effective amounts of one or more inhibitor of FGF signaling, one or more inhibitor of Notch signaling, and one or more inhibitor of MAPK/ERK kinase for about 4, 5, 6, 7, 8, 9, 10, 11, 12, 13, 14, 15, 16, 17, 18, 19, 20 or more days, wherein the cells are contacted with a concentration of said compounds effective to produce a population of cells expressing one or more markers of cortical neurons or precursors thereof.

In certain embodiments, the cells are contacted with an inhibitor of Notch signaling at a concentration of between about 1 and 20 μM, between about 2 and 18 μM, between about 4 and 16 μM, between about 6 and 14 μM, between about 8 and 12 μM, or about 10 μM, or about 5 μM.

In certain embodiments, the cells are contacted with an inhibitor of Notch signaling at a concentration of between about 2 and 20 μM, between about 4 and 20 μM, between about 6 and 20 μM, between about 8 and 20 μM, between about 10 and 20 μM, between about 12 and 20 μM, between about 14 and 20 μM, between about 16 and 20 μM, or between about 18 and 20 μM.

In certain embodiments, the cells are contacted with an inhibitor of Notch signaling at a concentration of between about 1 and 18 μM, between about 1 and 16 μM, between about 1 and 14 μM, between about 1 and 12 μM, between about 1 and 10 μM, between about 1 and 8 μM, between about 1 and 6 μM, between about 1 and 4 μM, or between about 1 and 2 μM.

In certain embodiments, the cells are contacted with an inhibitor of FGF signaling at a concentration of between about 0.5 and 20 μM, between about 1 and 18 μM, between about 2 and 16 μM, between about 4 and 14 μM, between about 6 and 12 μM, between about 8 and 10 μM, or about 2 μM, or about 5 μM, or about 10 μM.

In certain embodiments, the cells are contacted with an inhibitor of FGF signaling at a concentration of between about between about 0.5 and 18 μM, between about 0.5 and 16 μM, between about 0.5 and 14 μM, between about 0.5 and 12 μM, between about 0.5 and 10 μM, between about 0.5 and 8 μM, between about 0.5 and 6 μM, between about 0.5 and 4 μM, between about 0.5 and 2 μM, between about 0.5 and 1 μM..

In certain embodiments, the cells are contacted with an inhibitor of FGF signaling at a concentration of between about 1 and 20 μM, between about 2 and 20 μM, between about 4 and 20 μM, between about 6 and 20 μM, between about 8 and 20 μM, between about 10 and 20 μM, between about 12 and 20 μM, between about 14 and 20 μM, between about 16 and 20 μM, or between about 18 and 20 μM.

In certain embodiments, the cells are contacted with an inhibitor of MAPK/ERK kinase signaling at a concentration of between about 0.01 and 20 μM, between about 0.1 and 18 μM, between about 1 and 16 μM, between about 2 and 14 μM, between about 3 and 12 μM, between about 4 and 10 μM, between about 5 and about 8 μM, or about 0.4 μM, or about 1 μM, or about 8 μM.

In certain embodiments, the cells are contacted with an inhibitor of MAPK/ERK kinase signaling at a concentration of between about 0.01 and 18 μM, between about 0.01 and 16 μM, between about 0.01 and 14 μM, between about 0.01 and 12 μM, between about 0.01 and 10 μM, between about 0.01 and 8 μM, between about 0.01 and 6 μM, between about 0.01 and 4 μM, between about 0.01 and 2 μM, between about 0.01 and 1 μM.

In certain embodiments, the cells are contacted with an inhibitor of MAPK/ERK kinase signaling at a concentration of between about 0.1 and 20 μM, between about 1 and 20 μM, between about 2 and 20 μM, between about 4 and 20 μM, between about 6 and 20 μM, between about 8 and 20 μM, between about 10 and 20 μM, between about 12 and 20 μM, between about 14 and 20 μM, between about 16 and 20 μM, or between about 18 and 20 μM.

In certain embodiments, the present disclosure provides for a method of differentiating human stem cells into cortical neurons, or precursors thereof, wherein the cells are contacted with (i) an effective amount of an inhibitor of TGFβ/Activin-Nodal signaling (e.g., 10 μM), (ii) an effective amount of an inhibitor of BMP signaling (e.g., 250 nM), (iii) an effective amount of an inhibitor of Wnt signaling (e.g., 5 μM), (iv) an effective amount of an inhibitor of Notch signaling (e.g., 10 μM), (v) an effective amount of an inhibitor of FGF signaling (e.g., 5 or 10 μM), and (vi) an effective amount of an inhibitor of MAPK/ERK signaling (e.g., 1 or 8 μM). In certain embodiments, (iv), (v) and (vi) are contacted to the cells at least 2 days after the cells are initially contacted with an effective amount of (i).

In certain embodiments, the present disclosure provides for a method of differentiating human stem cells into cortical neurons, or precursors thereof, wherein the cells are contacted with (i) an effective amount of an inhibitor of TGFβ/Activin-Nodal signaling (e.g., 10 μM), (ii) an effective amount of an inhibitor of BMP signaling (e.g., 100 nM), (iii) an effective amount of an inhibitor of Wnt signaling (e.g., 2 μM), (iv) an effective amount of an inhibitor of Notch signaling (e.g., 5 μM), (v) an effective amount of an inhibitor of FGF signaling (e.g., 2 μM), and (vi) an effective amount of an inhibitor of MAPK/ERK signaling (e.g., 0.4 μM). In certain embodiments, (iv), (v) and (vi) are contacted to the cells at least 3 days after the cells are initially contacted with an effective amount of (i).

In certain embodiments, the concentration of inhibitors (i), (ii) and (iii) are decreased by about 10, 20, 30, 40, 50, 60 or 70% after contacting the cells for at least, or up to, 1, 2, 3, 4, 5, or 6 days.

In certain embodiments, the method further comprises subjecting said population of differentiated cells to conditions favoring maturation of said cells into a population of cortical neurons comprising contacting the cells with effective concentrations of one or bore activators of brain derived neurotrophic factor (BDNF), cAMP, and ascorbic acid signaling. In certain embodiments, the cells are contacted with said maturation compounds at least, or up to, about 5, 6, 7, 8, 9, 10, 11 or 12 days after initially contacting the cells with an effective concentration of (i), i.e., the one or more inhibitor of TGFβ/Activin-Nodal signaling.

In certain embodiments, the conditions favoring maturation comprises culturing the cells in a suitable cell culture medium. In certain embodiments, the suitable cell culture medium comprises a neurobasal (NB) medium. In certain embodiments, the suitable cell culture medium is an NB medium supplemented with L-Glutamine, and B27 (e.g., from Life Technologies).

In certain embodiments, the cells contacted according to the methods described herein express detectable levels of PAX6 (paired box 6) at least, or up to, about 4, 5, 6, 7 or 8 days or more after initially contacted with an effective amount of an inhibitor of TGFβ/Activin-Nodal signaling.

In certain embodiments, the cells are contacted with effective concentrations of (i) to (vi) for a period of time such that the cells express detectable levels of PAX6. In certain embodiments, said period of time is about 4, 5, 6, 7 or 8 days after the cells are initially contacted with an effective concentration of inhibitor (i), i.e., the one or more inhibitor of TGFβ/Activin-Nodal signaling.

In certain embodiments, the expression of PAX6 is detectable in at least about 50%, 60%, 70%, 80%, 90%, 95%, 98% or more of the cells of the cell population. In certain embodiments, the cells contacted according to the methods described herein express detectable levels of PAX6 about 6 days after initially contacted with an inhibitor of TGFβ/Activin-Nodal signaling.

In certain embodiments, the cells contacted according to the methods described herein express detectable levels of TUJ1 (class III beta-tubulin), FOXG1 (Forkhead Box G1), and/or DCX (doublecortin) at least, or up to, about 10, 11, 12, 13, 14, 15 or 16 days or more after initially contacted with an inhibitor of TGFβ/Activin-Nodal signaling.

In certain embodiments, the cells are contacted with effective concentrations of (i) to (vi) for a period of time such that the cells express detectable levels of TUJ1, FOXG1, and/or DCX. In certain embodiments, said period of time is about 10, 11, 12, 13, 14, 15 or 16 days after the cells are initially contacted with an effective concentration of inhibitor (i), i.e., the one or more inhibitor of TGFβ/Activin-Nodal signaling.

In certain embodiments, the cells contacted according to the methods described herein express detectable levels of TUJ1 about 13 days after initially contacted with an effective amount of an inhibitor of TGFβ/Activin-Nodal signaling.

In certain embodiments, the expression of TUJ1 is detectable in at least about 5%, 10%, 15%, 20%, 30%, 40%, 50%, 60%, 70%, 80%, 90%, 95%, 98% or more of the cells of the cell population. In certain embodiments, the cells further coexpress TBR1 (T-box, brain 1) and/or TLE4 (transducin like enhancer of split 4).

In certain embodiments, the cells contacted according to the methods described herein express detectable levels of TUJ1, wherein at least about 10%, 20%, 30%, 40%, 50%, 60%, 70%, 80%, 90% or more of the cells also expresses detectable levels of TBR1, TLE4, or a combination thereof.

In certain embodiments, the cells are contacted with effective concentrations of (i) to (vi) for a period of time such that the cells express detectable levels of TUJ1 and one or both of TBR1 and/or TLE4.

In certain embodiments, said cells expressing detectable levels of PAX6, TUJ1, FOXG1, DCX, TBR1, TLE4, or any combination thereof, is a proximate cortical neuron precursor.

In certain embodiments the cells contacted according to the methods described herein express a detectable level of a cortical neuron marker selected from the group consisting of TBR1 (T-box, brain 1), TLE4 (transducin like enhancer of split 4), DCX (doublecortin), RELN (reelin), CTIP2 (B-cell lymphoma/leukemia 11B), SATB2 (SATB homeobox 2), FOXP2 (forkhead box protein P2), RGS4 (regulator of G protein signaling 4), CUX2 (cut like homeobox 2), BLBP (brain lipid binding protein), and combinations thereof, at least, or up to, about 4, 5, 6, 7, 8, 9, 10, 11, 12, 13, 14, 15, 20, 25, 30, 33 days or more after initially contacted with an effective amount of inhibitor of TGFβ/Activin-Nodal signaling.

In certain embodiments, the expression of TBR1, TLE4, DCX, REELIN, CTIP2, SATB2, FOXP2, RGS4, CUX2, and/or BLBP is detectable in at least about 10%, 20%, 30%, 40%, 50%, 60%, 70%, 80%, 90% or more of the cells of the cell population. In certain embodiments, the cells also express detectable levels of TUJ1.

The presently disclosed subject matter also provides a population of in vitro differentiated cells produced by the methods described herein, and compositions comprising such in vitro differentiated cells.

In certain embodiments, the cells prepared according to the methods described herein exhibit electrophysiological properties of mature differentiated cortical neurons after at least, or up to, about 6, 7, 8, 9, 10, 11, 12, 13, 14, 15, 16, 17, 18, 19, 20 days or more after being contacted with an effective amount of an inhibitor of TGFβ/Activin-Nodal signaling.

In certain embodiments, the stem cells are contacted with effective amounts of inhibitors (i) through (iii) for at least about 2, 3, 4, 5, 6, 7, 8, 9, or 10 or more days, and are contacted with effective amounts of inhibitors (iv) through (vi) for at least about 2, 3, 4, 5, 6, 7, 8, 9, or 10 or more days, and are then further contacted for at least about 2, 3, 4, 5, 6, 7, 8, 9, or 10 or more days with: an effective amount of an activator of Wnt signaling, for example, a GSK30 inhibitor such as CHIR99021 (WO2011/149762; and Calder et al., J Neurosci. 2015 Aug. 19; 35(33):11462-81); an effective amount of an inhibitor of MAPK/ERK kinase; an effective amount of an inhibitor of Notch signaling; and/or an effective amount of an inhibitor of FGF signaling.

In certain embodiments, the method comprises (a) initially contacting human pluripotent stem cells with effective concentrations of (i) one or more inhibitor of transforming growth factor beta (TGFβ)/Activin-Nodal signaling, (ii) one or more inhibitor of bone morphogenetic protein (BMP) signaling, (iii) one or more inhibitor of Wnt signaling; (b) culturing said cells, for at least about six or seven days, with effective concentrations of (i) one or more inhibitor of transforming growth factor beta (TGFβ)/Activin-Nodal signaling, (ii) one or more inhibitor of bone morphogenetic protein (BMP) signaling, (iii) one or more inhibitor of Wnt signaling; (c) initially contacting said cells, at least about two or three days after (a), with effective concentrations of (iv) one or more inhibitor of MAPK/ERK kinase signaling, (v) one or more inhibitor of FGF signaling, and (vi) one or more inhibitor of Notch signaling; and (d) culturing said cells, for at least about ten or eleven days or until at least 20% of said cells express TUJ1, with effective concentrations of (iv) one or more inhibitor of MAPK/ERK kinase signaling, (v) one or more inhibitor of FGF signaling, and (vi) one or more inhibitor of Notch signaling.

In certain embodiments, the stem cells are contacted with effective amounts of inhibitors (i) through (iii) for at least about 2, 3, 4, 5, 6, 7, 8, 9, or 10 or more days, and are contacted with effective amounts of inhibitors (iv) through (vi) for at least about 2, 3, 4, 5, 6, 7, 8, 9, or 10 or more days, and are then further contacted for at least about 2, 3, 4, 5, 6, 7, 8, 9, or 10 or more days with an effective amount of one or more inhibitor of Notch signaling.

In certain embodiments, the above-described inhibitors, activators and molecules are added to a cell culture medium comprising the stem cells. Suitable cell culture media include, but are not limited to, Knockout® Serum Replacement (“KSR”) medium, N2 medium, and an ESSENTIAL 8™/ESSENTIAL 6™ (“E8/E6”) medium, and a Neurobasal (NB) medium (e.g., a NB medium supplemented with N2 and B-27® Supplement). KSR medium, N2 medium, E8/E6 medium and NB medium are commercially available.

KSR medium is a defined, serum-free formulation optimized to grow and maintain undifferentiated hESCs in culture. The components of a KSR medium are disclosed in WO2011/149762. In certain embodiments, a KSR medium comprises Knockout DMEM, Knockout Serum Replacement, L-Glutamine, Pen/Strep, MEM, and β-mercaptoethanol.

E8/E6 medium is a feeder-free and xeno-free medium that supports the growth and expansion of human pluripotent stem cells. E8/E6 medium has been proven to support somatic cell reprogramming. In addition, E8/E6 medium can be used as a base for the formulation of custom media for the culture of PSCs. One example E8/E6 medium is described in Chen et al., Nat Methods. 2011 May; 8(5):424-9, which is incorporated by reference in its entirety. One example of E8/E6 medium is disclosed in WO15/077648, which is incorporated by reference in its entirety. In certain embodiments, an E8/E6 cell culture medium comprises DMEM/F₁₂, ascorbic acid, selenum, insulin, NaHCO₃, transferrin, FGF2 and TGFβ. In certain embodiments, the E6 media does not include FGF2 and TGFβ. The E8/E6 medium differs from a KSR medium in that E8/E6 medium does not include an active BMP or Wnt ingredient. Thus, in certain embodiments, when an E8/E6 medium is used to culture the presently disclosed population of stem cells to differentiate into a population of cortical neurons, one or more inhibitor of BMP is not required to be added to the E8/E6 medium

N2 supplement is a chemically defined, animal-free, supplement used for expansion of undifferentiated neural stem and progenitor cells in culture. N2 Supplement is intended for use with DMEM/F₁₂ medium. The components of a N2 medium are disclosed in WO2011/149762. In certain embodiments, a N2 medium comprises a DMEM/F₁₂ medium supplemented with glucose, sodium bicarbonate, putrescine, progesterone, sodium selenite, transferrin, and insulin.

In certain embodiments, the stem cells are initially cultured in a KSR medium, or E6 medium, which is gradually replaced with increasing amount of a N2/B27 medium from about 1, about 2, about 3, about 4, about 5, about 6, about 7, about 8, about 9, about 10, about 11 or about 12 days after the initial contact of the stem cells with at least one of the above-described inhibitors, and activators. In certain embodiments, the stem cells are initially cultured in a KSR medium, which is gradually replaced with increasing amount of a N2/B27 medium from about day 4 after the initial contact of the stem cells with at least one of the above-described inhibitors and activators (e.g., 4 days after the initial contact of the stem cells with the one or more inhibitor of TGFβ/Activin-Nodal signaling). In certain embodiments, the stem cells are initially cultured in a E6 medium, which is gradually replaced with increasing amount of a N2/B27 medium from about day 5 after the initial contact of the stem cells with at least one of the above-described inhibitors and activators (e.g., 5 days after the initial contact of the stem cells with the one or more inhibitor of TGFβ/Activin-Nodal signaling).

The differentiated cortical neurons, or precursors thereof, can be purified after differentiation, e.g., in a cell culture medium. As used herein, the terms “purified,” “purify,” “purification,” “isolated,” “isolate,” and “isolation” refer to the reduction in the amount of at least one contaminant from a sample. For example, a desired cell type is purified by at least 10%, by at least 30%, by at least 50%, by at least 75%, by at least 90%, by at least 95%, by at least 99%, by at least 99.5%, or by at least 99.9% or more, with a corresponding reduction in the amount of undesirable cell types. The term “purify” can refer to the removal of certain cells (e.g., undesirable cells) from a sample. The removal or selection of non-cortical neuron cells, or precursors thereof, results in an increase in the percent of desired cells in the sample. In certain embodiments, the cells are purified by sorting a mixed cell population into cells expressing at least one cortical neuron marker. In certain embodiments, the cells are purified by sorting a mixed cell population into cells expressing at least one enteric cortical neuron marker, e.g., TBR1, TLE4, DCX, REELIN, CTIP2, SATB2, FOXP2, RGS4, CUX2, BLBP, or combinations thereof.

### 5.3. Compositions Comprising Differentiated Cell Populations

The present disclosure also provides for a population of in vitro differentiated cells expressing one or more neuronal marker, for example, a cortical neuron marker, or precursor cells thereof, prepared according to the methods described herein. In certain embodiments, at least about 70% (e.g., at least about 75%, at least about 80%, at least about 85%, at least about 90%, at least about 95%, or at least about 99%, or at least about 99.5%, or at least 99.9%) of the population of cells express one or more cortical neuron marker, for example, TBR1, TLE4, DCX, REELIN, CTIP2, SATB2, FOXP2, RGS4, CUX2, BLBP, or combinations thereof.

In certain embodiments, less than about 15% (e.g., less than about 10%, less than about 5%, less than about 4%, less than about 3%, less than about 2%, less than about 1%, less than about 0.5%, or less than about 0.1%) of the population of cells express one or more marker selected from the group consisting of stem cell markers (e.g., OCT4 (octamer-binding transcription factor 4), NANOG (Nanog homeobox), SOX2 (SRY-Box 2), LIN28 (Lin-28 homolog A), SSEA4 (Stage-specific embryonic antigen-4) and/or SSEA3 (Stage-specific embryonic antigen-3), glial cell markers (e.g., GFAP (Glial fibrillary acidic protein), AQP4 (Aquaporin 4), and/or OLIG2 (Oligodendrocyte Lineage Transcription Factor 2)), retinal cell markers (e.g., CHX10 (Visual System Homeobox 2)), peripheral sensory neurons (e.g., BRN3A (brain-specific homeobox/POU domain protein 3A), and/or ISL1 (ISL LIM Homeobox 1)), neural crest precursors (e.g., SOX10 (SRY-Box 10)), or cranial placode precursors (e.g., SIX1 (SIX Homeobox 1)).

In certain embodiments, the differentiated cell population is derived from a population of human stem cells. The presently disclosed subject matter further provides for compositions comprising such differentiated cell population.

In certain embodiments, the composition comprises a population of from about 1×10⁴ to 5 about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed stem-cell-derived cells.

In certain non-limiting embodiments, the composition further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that comprises a pharmaceutically acceptable carrier, excipient, diluent or a combination thereof. The compositions can be used for preventing and/or treating CNS neurodegenerative disorders, as described herein.

### 5.4 Methods of Preventing and/or Treating CNS Neurodegenerative Disorders

The in vitro differentiated cells that express one or more cortical neuron marker (also referred to as “stem-cell-derived cortical neurons”), or precursors thereof, can be used for preventing and/or treating a neurodegenerative disorder. The presently disclosed subject matter provides for methods of preventing and/or treating a neurodegenerative disorder comprising administering an effective amount of the presently disclosed stem-cell-derived cortical neurons, and precursors thereof, into a subject suffering from a neurodegenerative disorder. Non-limiting examples of neurodegenerative disorder include Parkinson's disease, Alzheimer's disease, and schizophrenia.

The presently disclosed stem-cell-derived cortical neurons, and precursors thereof, can be administered or provided systemically or directly to a subject for treating or preventing a neurodegenerative disorder. In certain embodiments, the presently disclosed stem-cell-derived cortical neurons, and precursors thereof, are directly injected into an organ of interest (e.g., the central nervous system (CNS)).

The presently disclosed stem-cell-derived cortical neurons, and precursors thereof, can be administered in any physiologically acceptable vehicle. Pharmaceutical compositions comprising the presently disclosed stem-cell-derived cells and a pharmaceutically acceptable carrier are also provided. The presently disclosed stem-cell-derived cortical neurons, and precursors thereof, and the pharmaceutical compositions comprising thereof can be administered via localized injection, orthotopic (OT) injection, systemic injection, intravenous injection, or parenteral administration. In certain embodiments, the presently disclosed stem-cell-derived cortical neurons, and precursors thereof, are administered to a subject suffering from a neurodegenerative disorder via orthotopic (OT) injection.

The presently disclosed stem-cell-derived cortical neurons, and precursors thereof, and the pharmaceutical compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed stem-cell-derived cortical neurons, and precursors thereof.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived cortical neurons, and precursors thereof. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the presently disclosed stem-cell-derived cortical neurons, and precursors thereof, is the quantity of cells necessary to achieve an optimal effect. An optimal effect includes, but are not limited to, repopulation of CNS regions of a subject suffering from a neurodegenerative disorder, and/or improved function of the subject's CNS.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the neurodegenerative disorder, or otherwise reduce the pathological consequences of the neurodegenerative disorder. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the presently disclosed stem-cell-derived cortical neurons, and precursors thereof, is an amount that is sufficient to repopulate CNS regions of a subject suffering from a neurodegenerative disorder. In certain embodiments, an effective amount of the presently disclosed stem-cell-derived cortical neurons, and precursors thereof, is an amount that is sufficient to improve the function of the CNS of a subject suffering from a neurodegenerative disorder, e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's CNS.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the presently disclosed stem-cell-derived cells.

### 5.5 Kits

the presently disclosed subject matter provides for kits for inducing differentiation of stem cells. In certain embodiments, the kit comprises one or more of the following: (a) one or more inhibitor of transforming growth factor beta (TGFβ)/Activin-Nodal signaling, (b) one or more inhibitor of BMP signaling, (c) one or more inhibitor of Wnt signaling (d) one or more inhibitor of FGF signaling, (e) one or more inhibitor of Notch signaling, (f) one or more inhibitor of MAPK/ERK kinase signaling, and (g) instructions for inducing differentiation of the stem cells into a population of differentiated cells that express one or more neuronal marker, for example, a cortical neuron marker, or precursor cells thereof, according to the methods described herein.

The presently discloses subject matter also provides for kits comprising a population of differentiated cells that express one or more neuronal marker, for example, a cortical neuron marker, or precursor cells thereof, wherein the cells are prepared according to the methods described herein. In certain embodiments, the cells are comprised in a pharmaceutical composition.

### 6. Example

The presently disclosed subject matter will be better understood by reference to the following Example, which is provided as exemplary of the presently disclosed subject matter, and not by way of limitation.

6.1 EXAMPLE 1: Methods of Preparing Stem Cell-Derived Cortical Neurons by Contacting a Population of Stem Cells with Inhibitors of 6 Signal Transduction Pathways.

### SUMMARY

Considerable progress has been made in converting human pluripotent stem cells (hPSCs) into functional neurons. However, the protracted timing of human neuron specification and functional maturation remains a key challenge which hampers the routine application of hPSC-derived lineages in disease modeling and regenerative medicine. Using a combinatorial small molecule screen we have previously identified conditions for the rapid induction of peripheral sensory neurons. Here we report on the rapid induction of cortical neurons in an effort to generalize the approach for accelerated access to CNS fates. We demonstrate the combinatorial use of 6 pathway inhibitors to induce post mitotic cortical neurons by day 13 and functional electrophysiological properties by day 16 of differentiation without the need for glial co-culture. Neurons transplanted at day 8 of differentiation into the postnatal mouse cortex are functional and establish long-distance projections as illustrated using iDISCO-based whole brain imaging. Accelerated differentiation into cortical neuron fates should facilitate hPSC-based strategies for disease modeling and cell therapy in CNS disorders.

### Results

In an effort to accelerate neuronal fate acquisition the disclosed subject matter provides the use of two additional small molecules: SU5402, a potent inhibitor of fibroblast growth factor (FGF) signaling and DAPT, a 7-secretase inhibitor blocking Notch signaling⁶. The combinatorial application of those two inhibitors (SD) with dual SMAD inhibition and WNT activation yields 75% post-mitotic neurons by day 11 of differentiation⁷, the same time period required for neural precursor cell induction under standard dual-SMAD inhibition conditions¹. However, co-expression of BRN3A and ISL1 in those rapidly-induced neurons defined them as peripheral sensory rather than PAX6-derived CNS neurons⁷. Therefore it has remained unclear whether strategies to accelerate neuronal fate acquisition during sensory fate specification can be adapted for CNS fates. PAX6-derived cortical neurons are of particular interest for studies in human development and for modeling human neurodevelopmental and neurodegenerative CNS disorders. While reliable protocols exist to derive cortical neurons from hPSCs, those conditions require between 30-90 days of differentiation from hPSCs to yield both lower and upper layer cortical neurons^{15, 30} and even more protracted time periods to achieve full maturation. Here we aim to identify small-molecule based conditions that greatly accelerate human cortical neuron fate induction to facilitate the routine application of hPSC-derived neurons in applications for disease modeling and regenerative medicine.

Development of an Accelerated CNS Neuron Differentiation Protocol

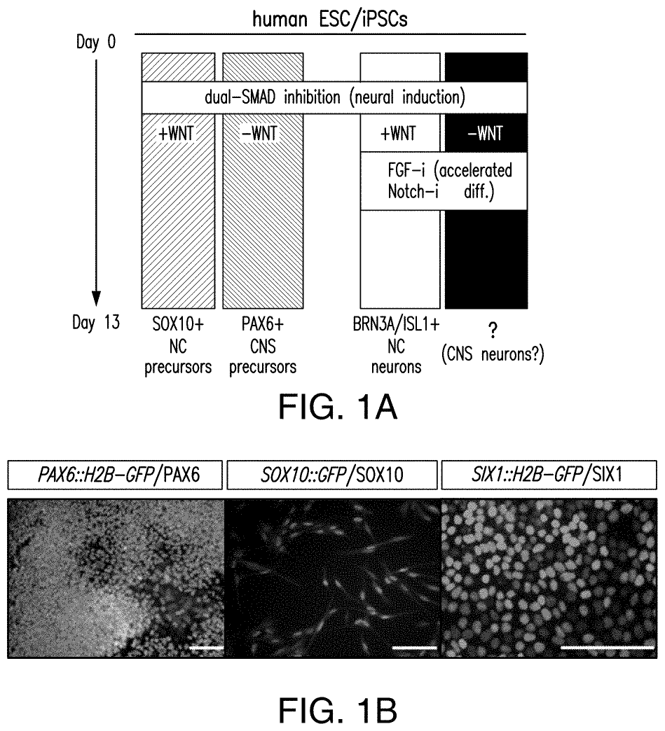

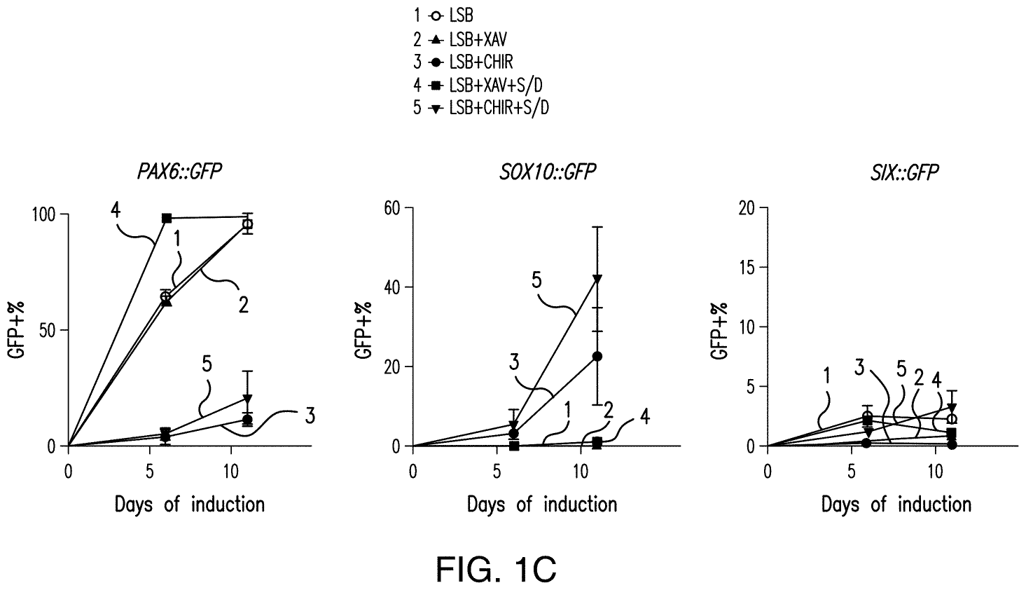

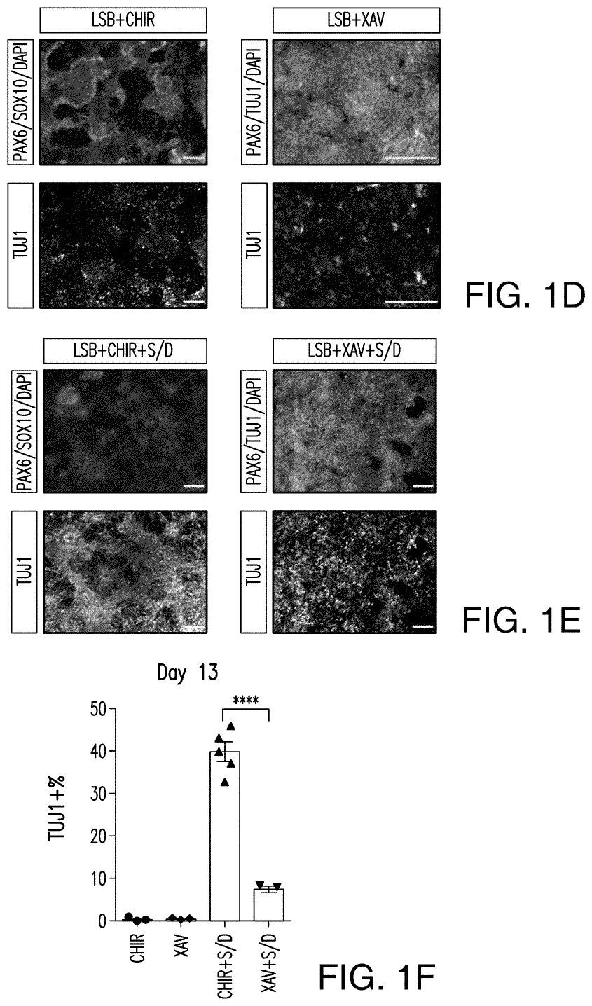

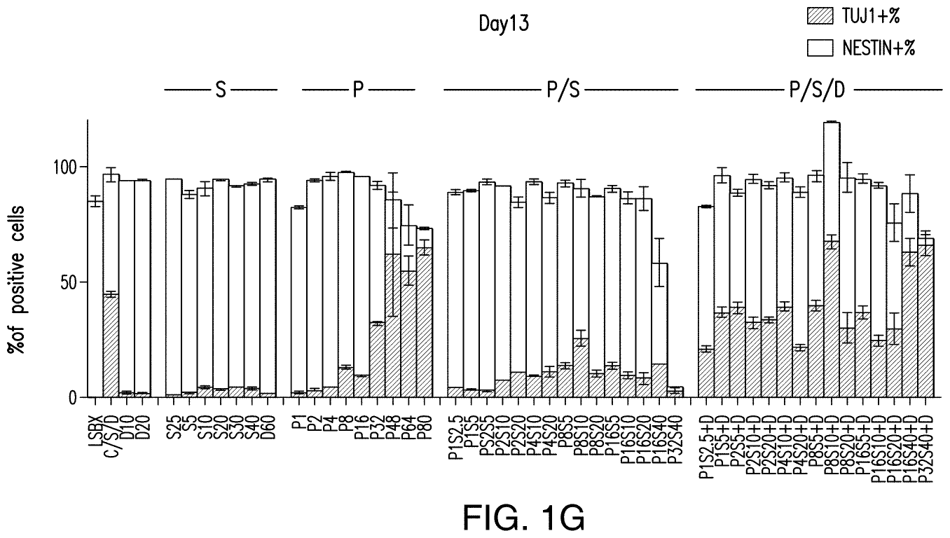

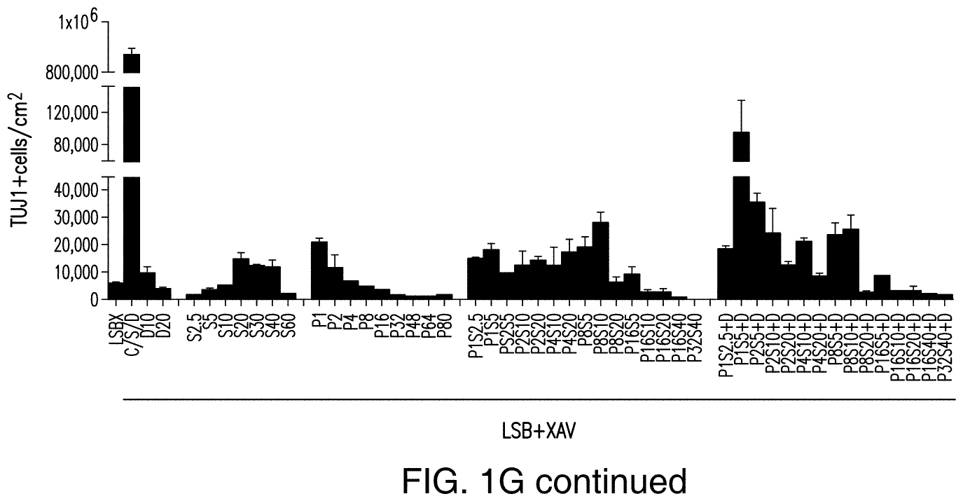

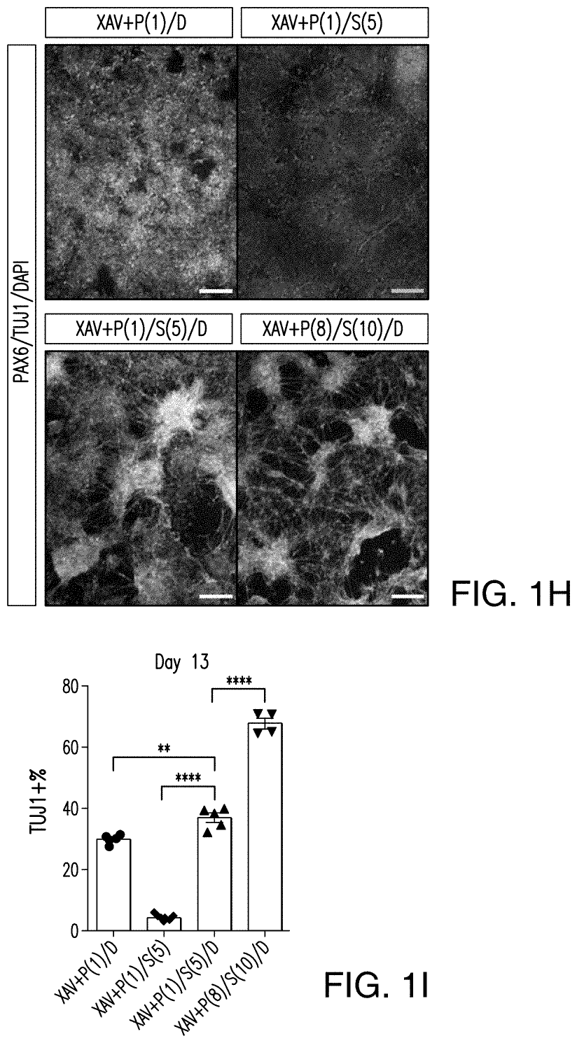

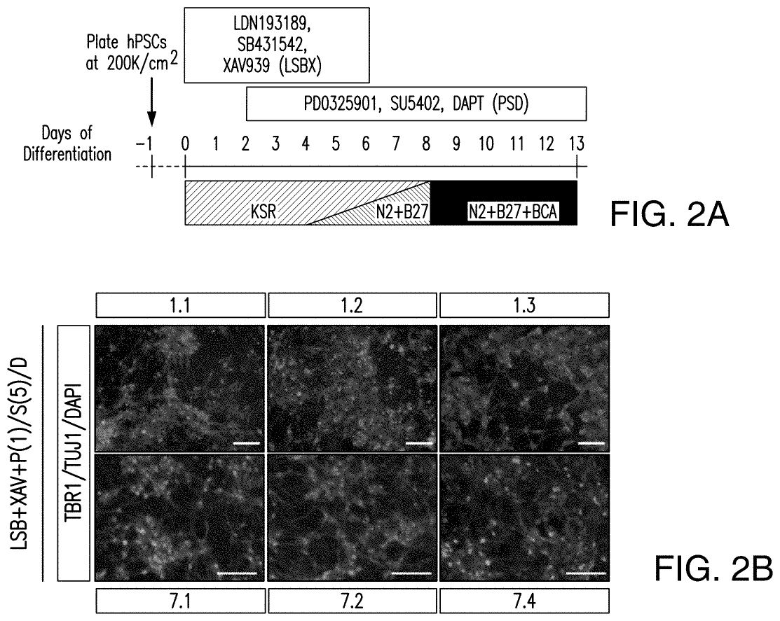

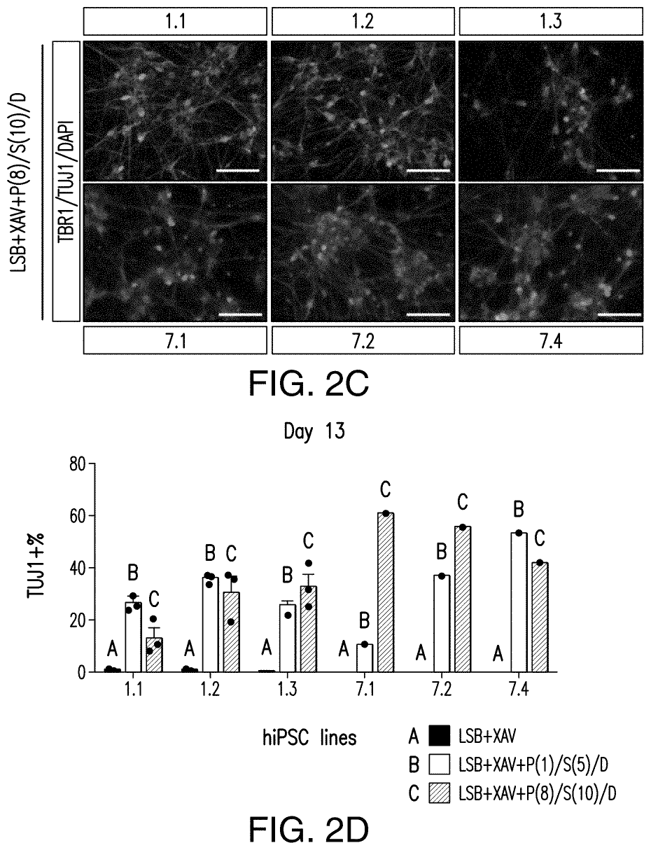

Given the critical roles of WNT signaling in determining CNS versus neural crest fate^{3, 8}, the disclosed methods developed a combinatorial small molecule approach that inhibits rather than activates WNT signaling that triggered rapid differentiation into cortical neuron fates (FIG. 1A). The disclosed methods exchange the GSK30 inhibitor CHIR99021 (C; WNT agonist) with the WNT antagonist XAV939, which acts via tankyrase inhibition and stabilization of axin⁹. All other inhibitors (LDN+SB=dual SMAD inhibition (LSB), SU5402 and DAPT) can remain unchanged for those initial studies aimed at rapidly inducing forebrain neuron fates (LSB+X/S/D protocol, FIG. 2A). Given the inventors' experience in unexpectedly triggering a CNS to PNS fate switch during rapid neuronal induction⁷, the disclosed methods first assessed the impact on early ectodermal lineage choice under X/S/D conditions using three genetic hESC reporter lines. For monitoring CNS lineage the disclosed methods establish a novel PAX6::H₂B-GFP reporter line, for neural crest fate the disclosed methods used a prior published SOX10::GFP reporter line^{2, 7} and for cranial placode identity the disclosed methods used a novel SIX1::H2B-GFP line. The PAX6 and SIX1 reporter lines were generated both using TALEN-based gene targeting. The disclosed methods validated the faithfulness of the reporters after in vitro differentiation,^{2, 10} by matching GFP with corresponding protein expression using immunocytochemistry (FIG. 1B).

The disclosed methods also assessed ectodermal fate choice under the X/S/D conditions, as illustrated in FIG. 1C. Consistent with previous work, both LSB and LSB+X gave rise to a near uniform population (>95%) of PAX6+ cells with very few SOX10+ or SIX1+ contaminants. In contrast, LSB+C or LSB+C/S/D (also referred to as 3i or PNS sensory neuron protocol⁷) gave rise to only few PAX6+ but large numbers of SOX10+ neural crest precursors compatible with an important role for WNT signaling in neural crest induction⁷. Importantly, LSB+X/S/D, our candidate rapid CNS neuron protocol, gave rise to almost pure population (>98%) of PAX6+ cells as early as day 6 of differentiation. This is significantly faster than LSB and LSBX, compatible with a role for FGF inhibition in exiting pluripotency in hPSCs¹¹. Resulting data demonstrate CNS identity and accelerated timing of neural induction following exposure to SU5402 and DAPT, as illustrated in FIG. 1C.

The disclosed methods also assessed whether LSB+X/S/D can induce putative CNS neurons with efficiencies comparable to those reported for the 3i protocol⁷ (LSB+C/S/D) during sensory neuron fate specification. Using intracellular flow cytometry for j-III tubulin (TUJ1), a pan-neuronal marker, LSB+C and LSB+X were largely devoid of TUJ1+ neurons by day 13 while 3i conditions (LSB+C/S/D) resulted in 40% TUJ1+ cells. In contrast, the novel LSB+X/S/D condition gave rise to only 10% TUJ1+ neurons, as illustrated in FIGS. 1D-1F. These data indicate that inhibition of WNT signaling in the presence of SU5402 and DAPT yields CNS lineage neurons but only at moderate efficiency.

In an effort to enhance neuronal conversion efficiency, the disclosed methods perform a candidate small molecule screen in LSB+X. The disclosed methods can select molecules targeting signaling pathways involved in neural precursor cell proliferation such as SHH (Cyclopamine, Cur-61414, Purmorphamine), PI3K and PDGFR (LY-294002, Imatinib), MYC/bromodomain proteins (JQ1), retinoid signaling (all-trans retinoic acid), TGFβ activation (IDE-1), HMG-CoA reductase inhibition (Lovastatin) and the nicotinamide phosphoribosyltransferase inhibition (P7C3) as well as signaling pathways downstream of FGF receptor activation, including ERK signaling (PD0325901). Inhibition of ERK1/2 in the mouse causes premature neuronal differentiation during cortical development¹² and ERK inhibition has been previously proposed as a strategy to enhance overall neuronal differentiation in hPSCs¹³. Most conditions did not yield a significant improvement in neuronal fate acquisition (data not shown).

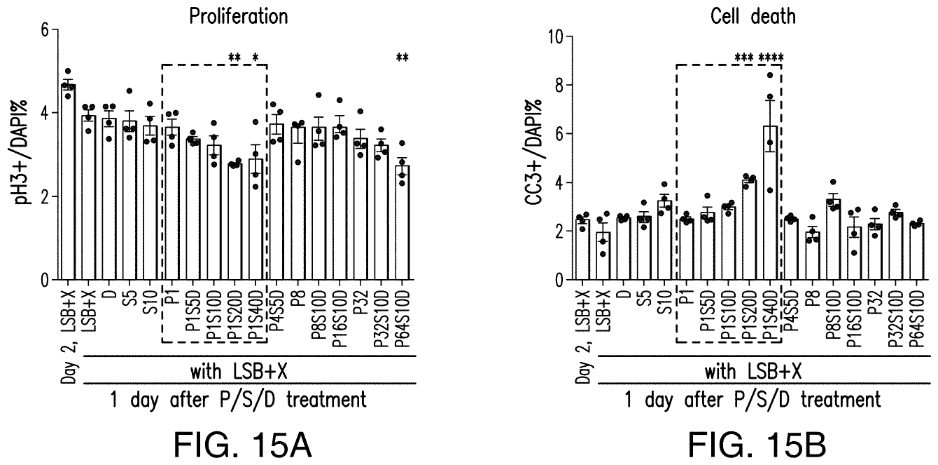

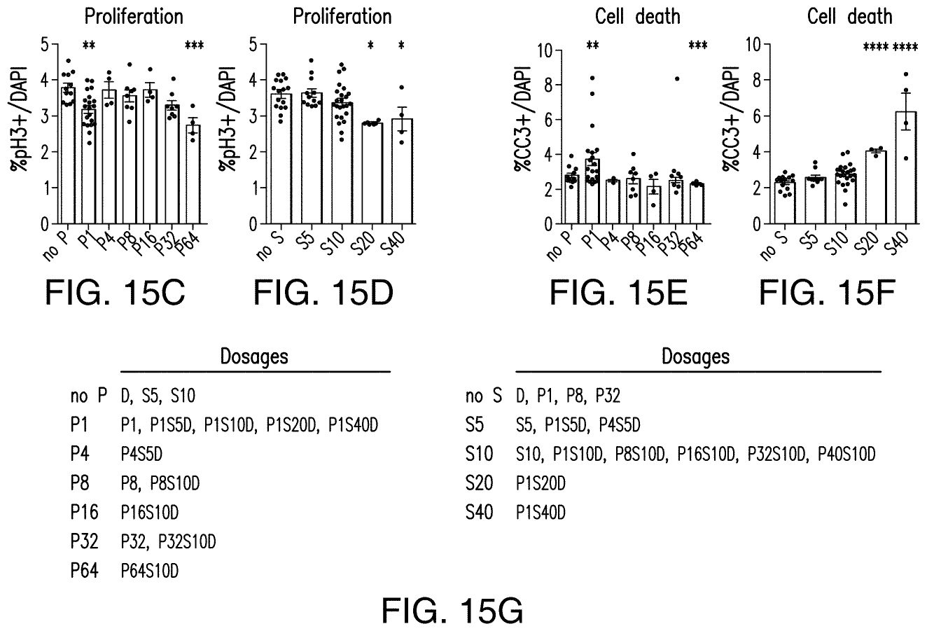

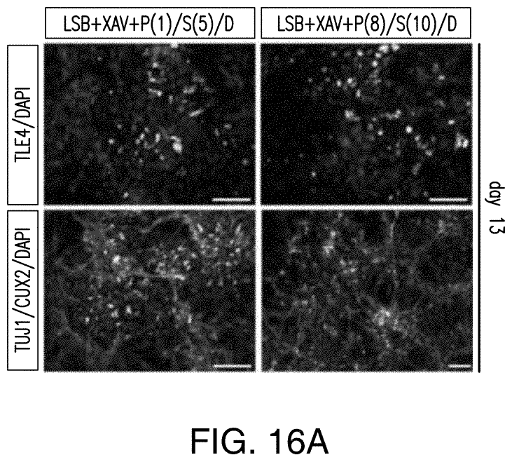

However, PD0325901, an orally bio-available, potent inhibitor for mitogen-activated protein kinase (MAPK/ERK kinase or MEK)⁸, could boost the yield of TUJ1+% to >50%, a value comparable to the 3i sensory neuron protocol, when used at high concentrations (FIG. 1G, upper panel). However, the high percentage of TUJ1+ cells was correlated with a low yield in total cell numbers suggesting toxicity (FIG. 1G, lower panel). Increased total neuron yields were obtained when lower PD concentrations were combined with SU exposure in an effort to balance neuronal induction efficiency and overall cell toxicity. Additional treatment with DAPT did not negatively affect overall neuron yield but further increased the efficiency of neuronal induction with several conditions achieving >50% TUJ1+ cells at day 13 of differentiation. These data define LSB+X+P/S/D as a promising candidate condition for the rapid induction of cortical neurons. To understand whether reduced yield is due to rapid cell cycle exit or direct toxicity, we measured phospho-Histone 3 (pH3) and cleaved Caspase 3 (CC3) as markers of cell proliferation and death, respectively. Exposure to both PD0325901 and SU5402 reduced cell proliferation as early as 24 hrs after P/S treatment, while cell death was observed only at high doses of SU5402 (FIG. 15A-F), implicating restriction of precursor cell proliferation as a key factor in the rapid neuronal differentiation response. The two concentrations that yielded high percentages of neurons with only low levels of toxicity include: i) P (1 μM) S (5M)=P1S5D condition and ii) P (8 μM) S (10 μM)=P8S10D condition (FIGS. 1H and 1I).

Phenotypic Analysis of CNS and Cortical Neuron Identity

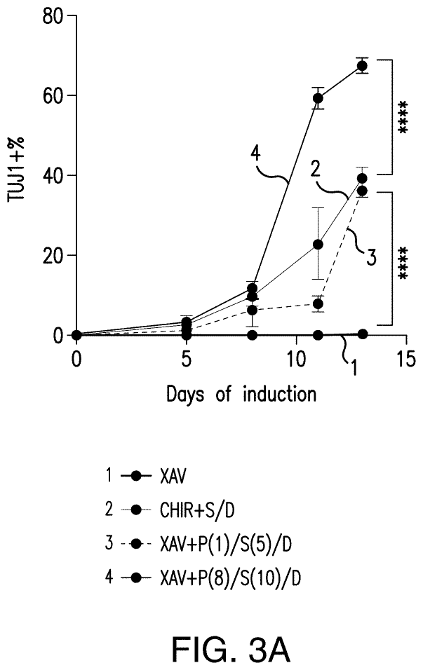

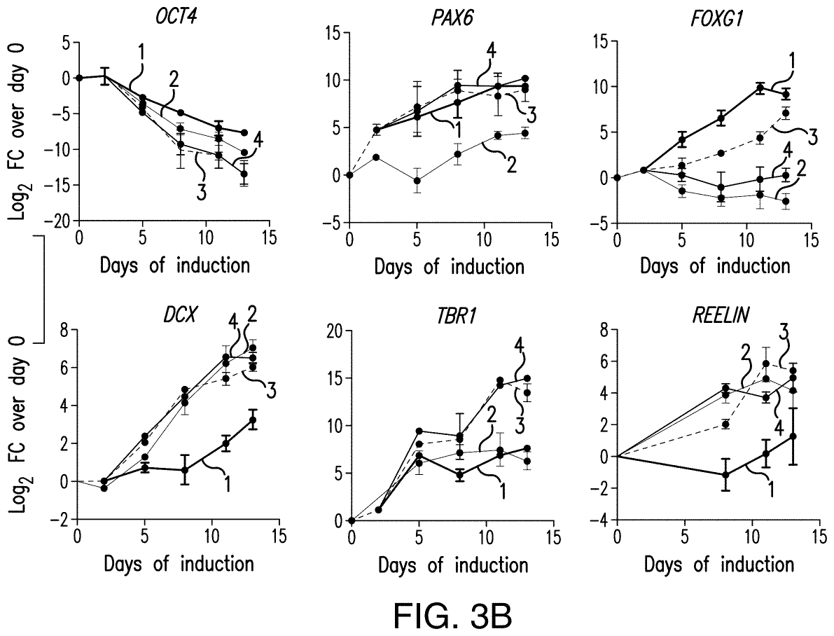

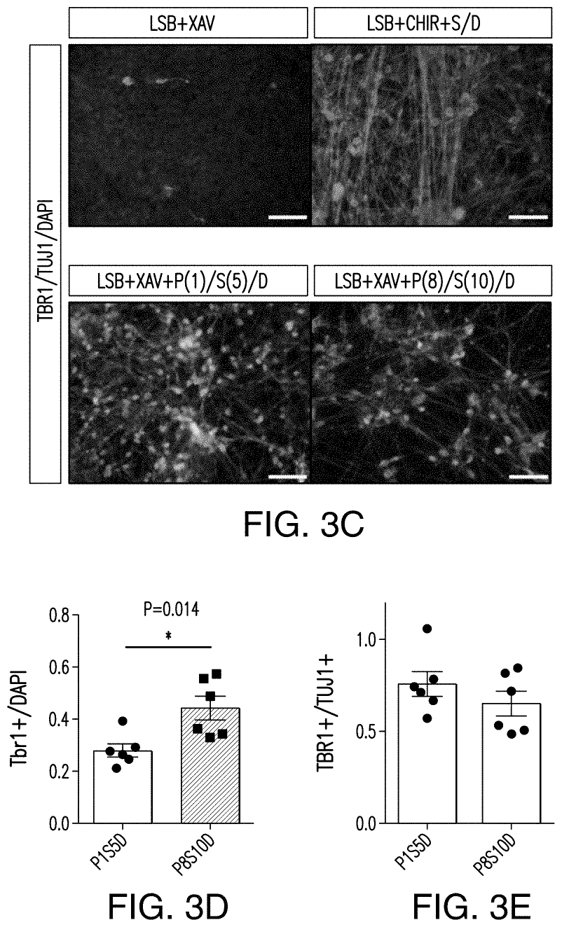

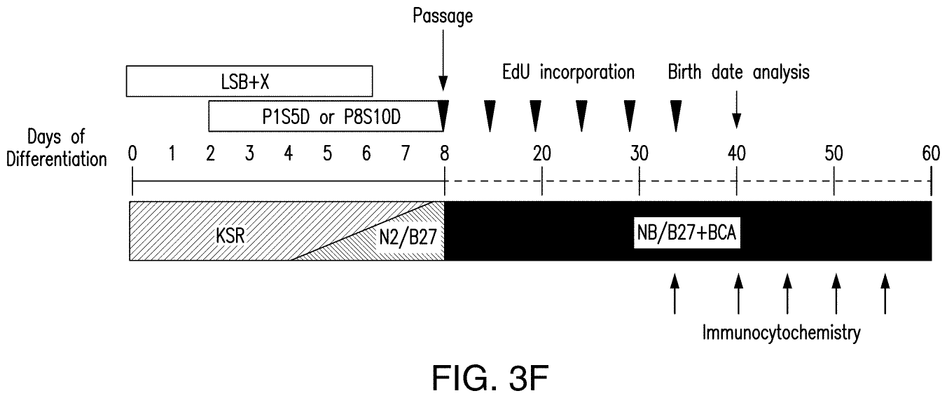

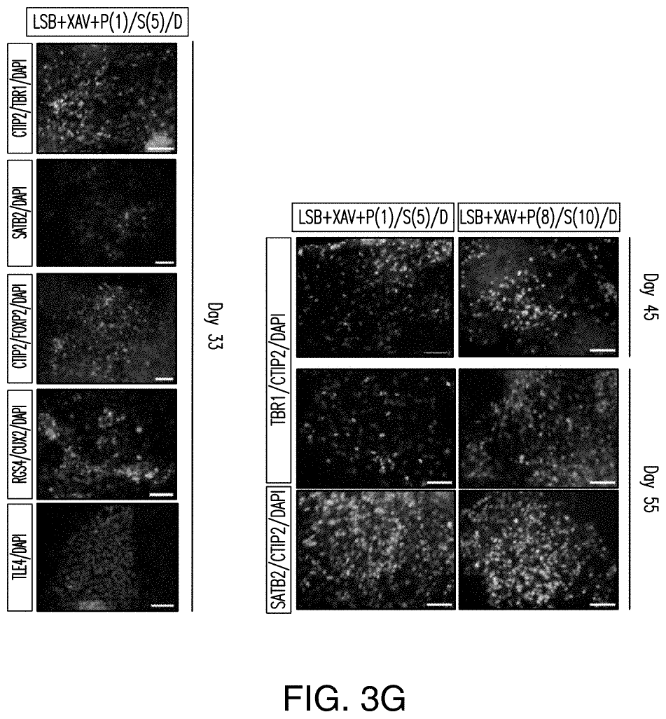

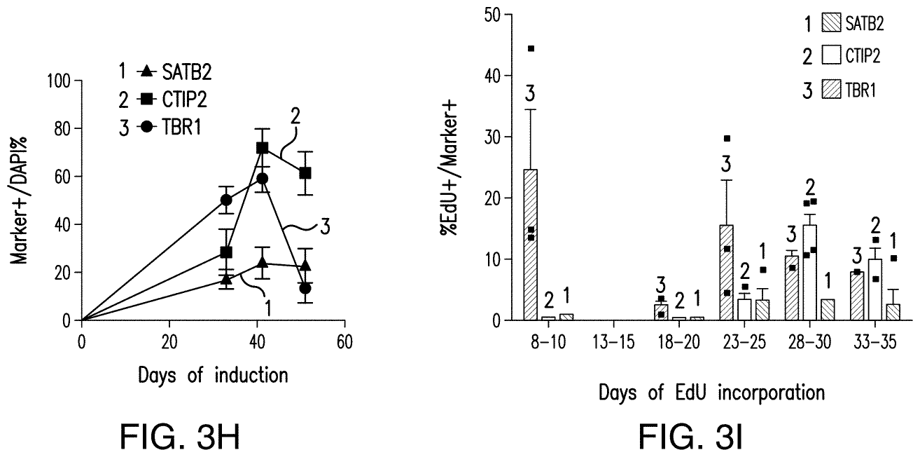

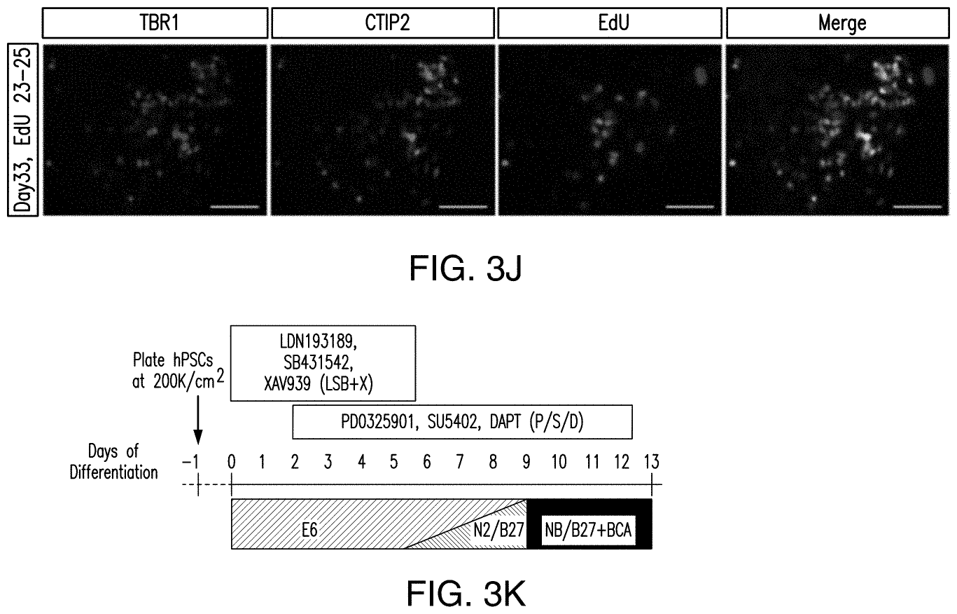

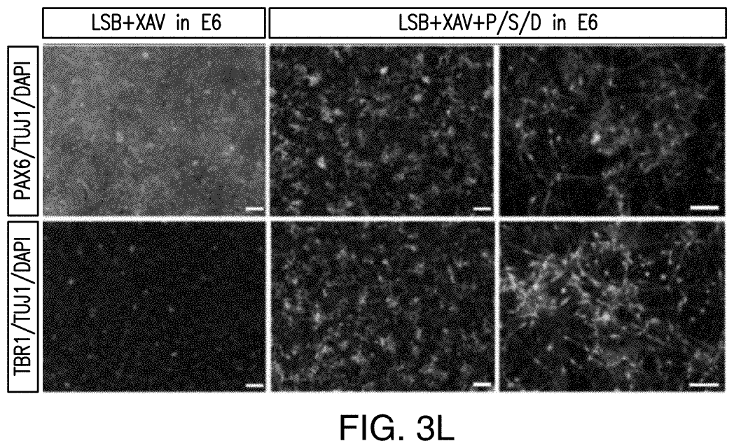

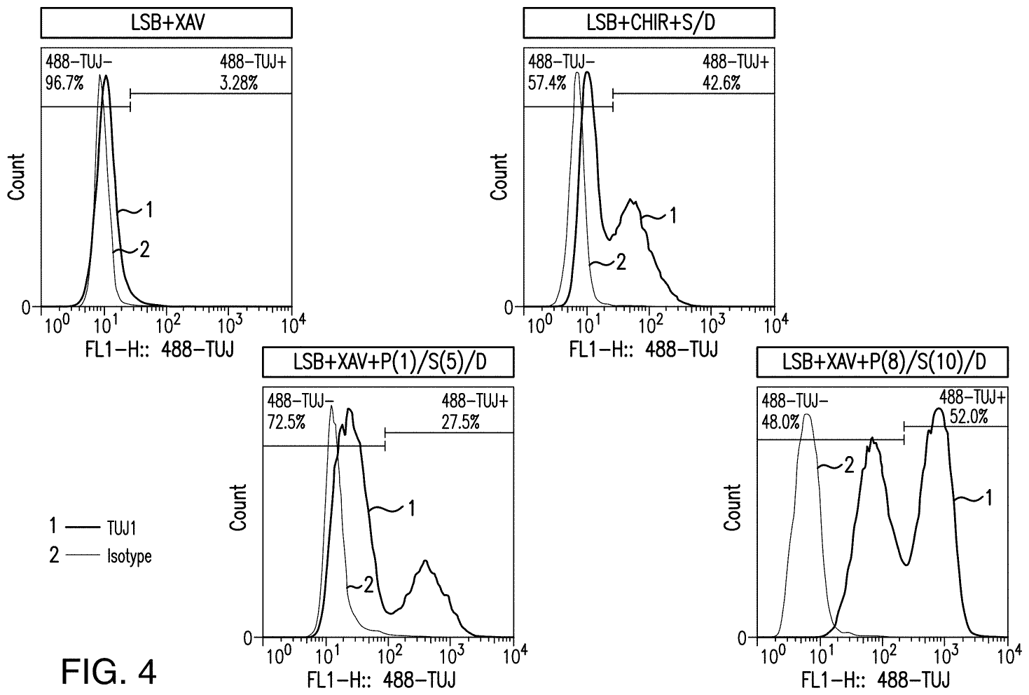

Temporal flow analysis demonstrated that the P8S10D condition resulted in a dramatic acceleration of neuronal fate acquisition, as illustrated in FIG. 3A and FIG. 4, with significantly higher percentage of neurons (70% of TUJ1+ neurons) at day 13 compared to 3i (C+S/D) or the milder CNS (P1S5D) neuron protocol. Gene expression analysis confirmed downregulation of the pluripotency marker OCT4 and induction of neural and neuronal markers PAX6, FOXG1 and DCX, as well as markers of early born cortical neurons, including TBR1 (preplate, subplate and layer VI) and REELIN, in LSB+X/P/S/D conditions. In contrast, the sensory neuron (3i) and the CNS protocol without acceleration (LSB+X) showed lack of cortical and neuronal marker induction respectively (FIG. 3B).

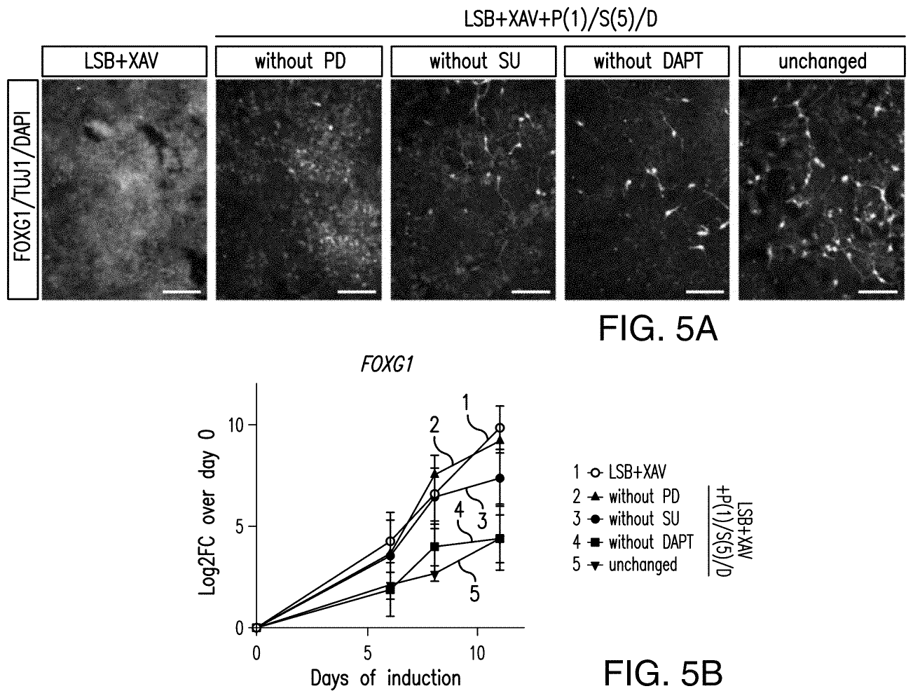

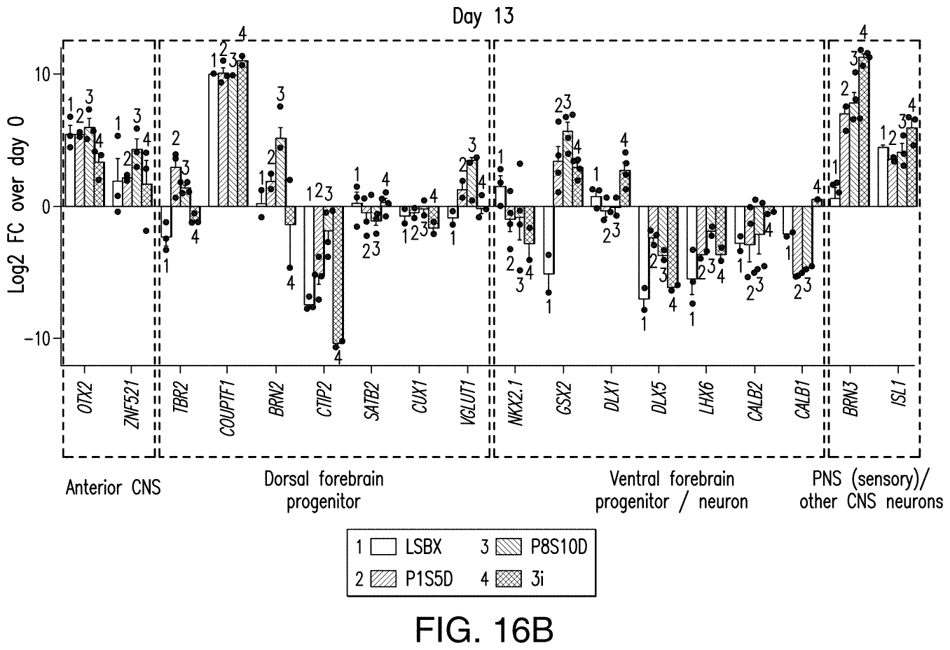

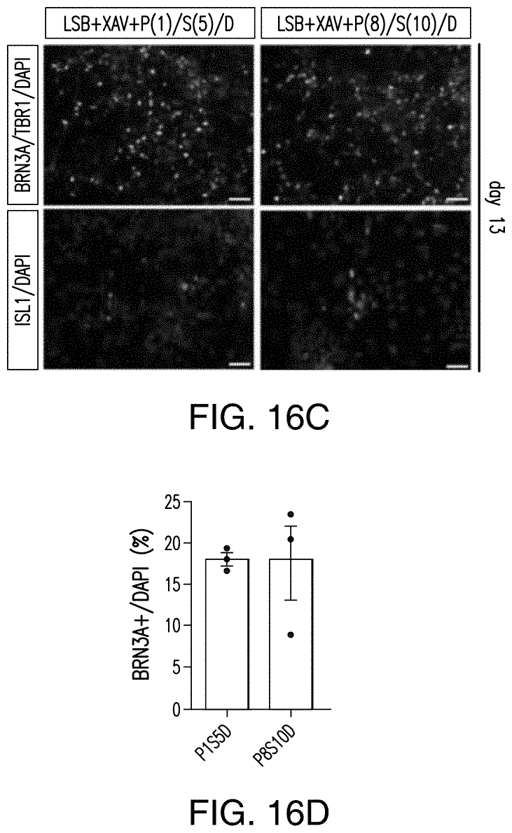

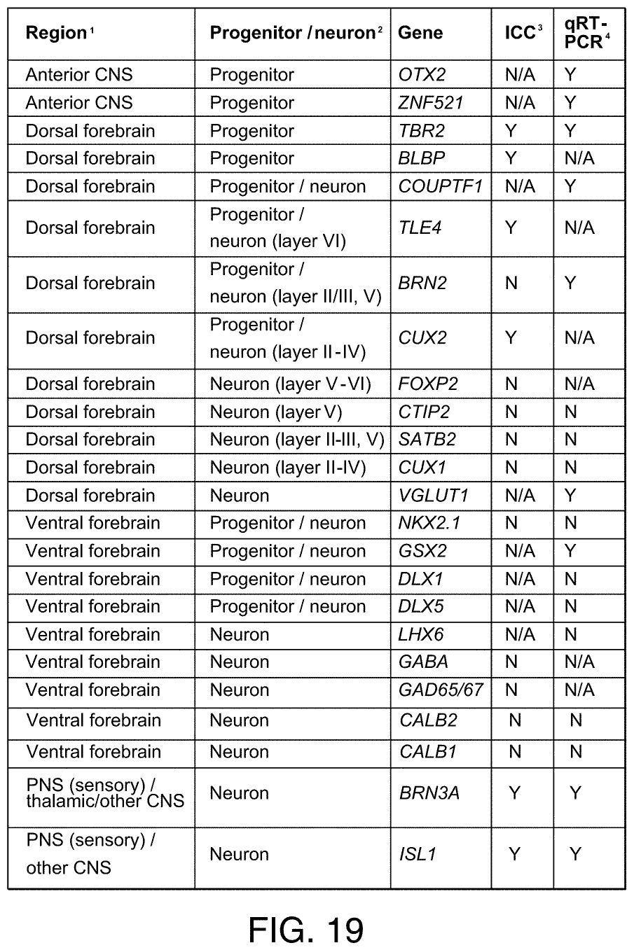

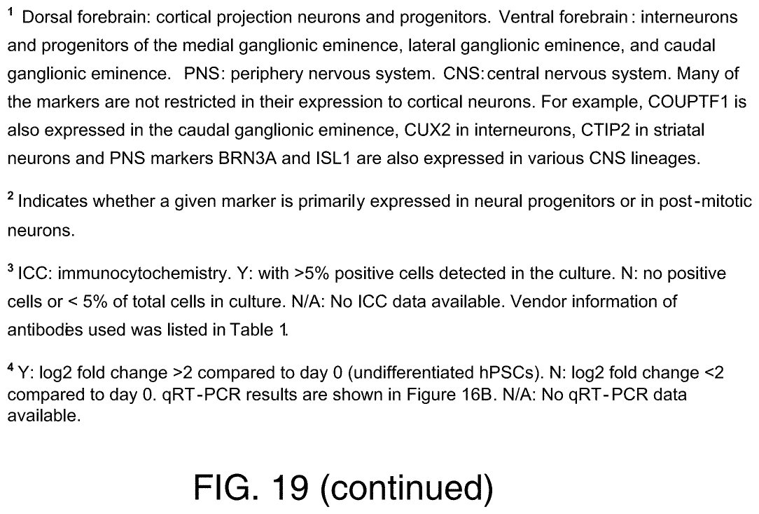

Given the limited induction of the forebrain marker FOXG1 under P8S10D conditions, the impact of each small molecule on FOXG1 expression (FIGS. 5A and 5B) was tested. Those data showed that in particular exposure to PD during neural induction reduced the efficiency of FOXG1 induction. However, highly efficient induction of layer VI cortical neuron marker TBR1+ was observed under both P1S5D and P8S10D conditions with co-expression in more than 50% of all TUJ1+ cells at day 13 (FIGS. 3C-3E) with similar expression of the layer VI marker TLE4 (FIG. 16A). To identify the remaining that were negative for TBR1 and TLE4, we screened a panel of additional markers at day 13 (FIG. 19, FIG. 16B). Surprisingly, 15%-20% neurons expressed BRN3A but only very few neurons expressed ISL1, suggesting the presence of a contaminating BRN3A+ CNS lineage (FIG. 19, FIG. 16C-D). Based on the expression of both BRN3A and GSX2 (FIG. 16B) those non-cortical neurons may correspond to an early thalamic lineage (www.gensat.org; http://developingmouse.brain-map.org). Characterization of the TUJ1-negative fraction showed the presence of cells positive for TBR2, BLBP and CUX2 (FIG. 19, FIG. 16A-B), consistent with cortical precursor cell identity. We further observed upregulation (compared to LSB+X) of other anterior CNS and cortical progenitor and neuron markers in LSB+X/P/S/D conditions (FIG. 16B). However, we did not detect the expression of ventral forebrain, cortical interneuron or other GABAergic neuron fates (FIG. 19, FIG. 16B).

To determine whether LSB+X/P/S/D conditions are robust across multiple lines, we tested 6 independent hiPSC lines derived from two healthy individuals. By 13 days of differentiation, all lines were enriched for TBR1+/TUJ1+ neurons and displayed morphologies similar to those obtained from WA09 hESC line (FIGS. 2B and 2C). Quantification of the percentages of neuron showed similar efficiency to that observed for WA09, though there was some variability across lines (FIG. 2D). In an effort to translate the accelerated protocol to GMP-compatible culture conditions, the protocol was further adapted to an ESSENTIAL6™ medium (E6)-based induction platform (FIG. 3K). We observed efficient PAX6 induction and accelerated generation of highly enriched populations of TBR1+ post-mitotic neurons by 13 days of differentiation (FIG. 2H). Thus, the rapid induction strategy can be applied across hiPSC lines and adapted to GMP-compatible culture conditions.

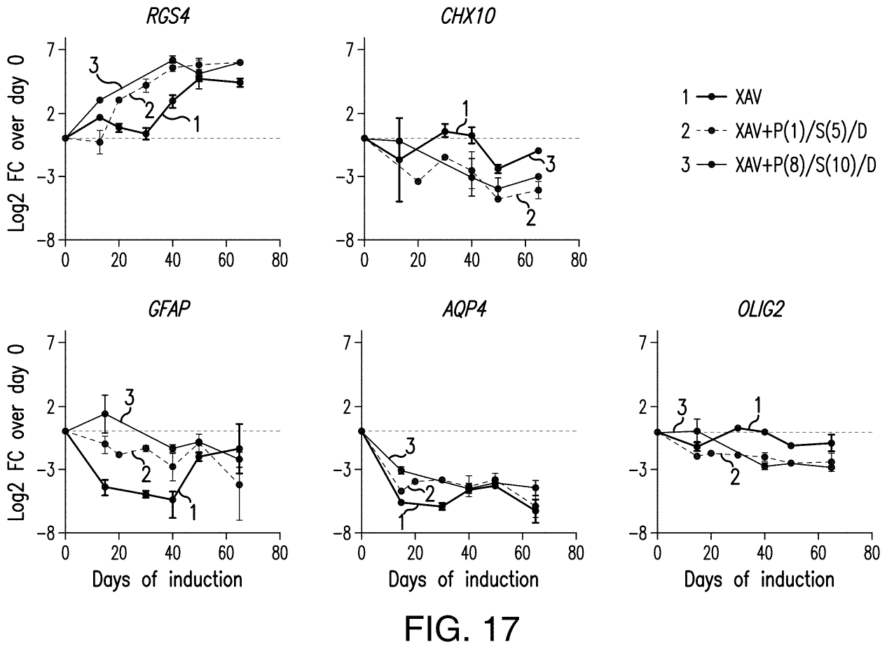

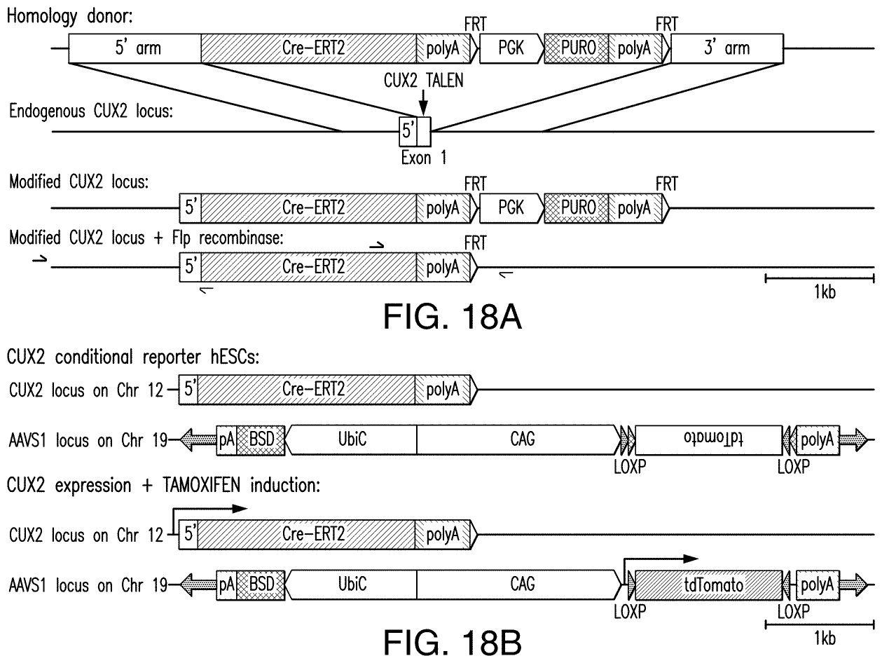

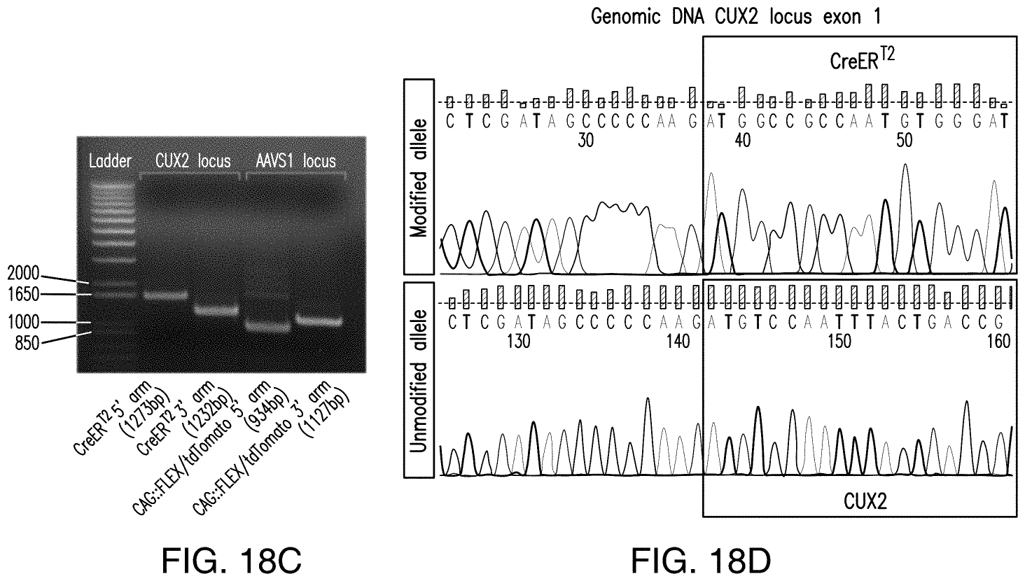

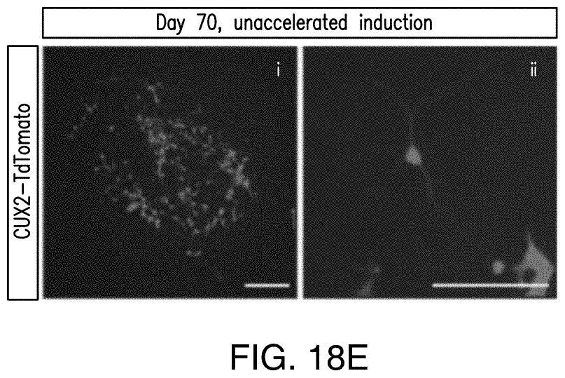

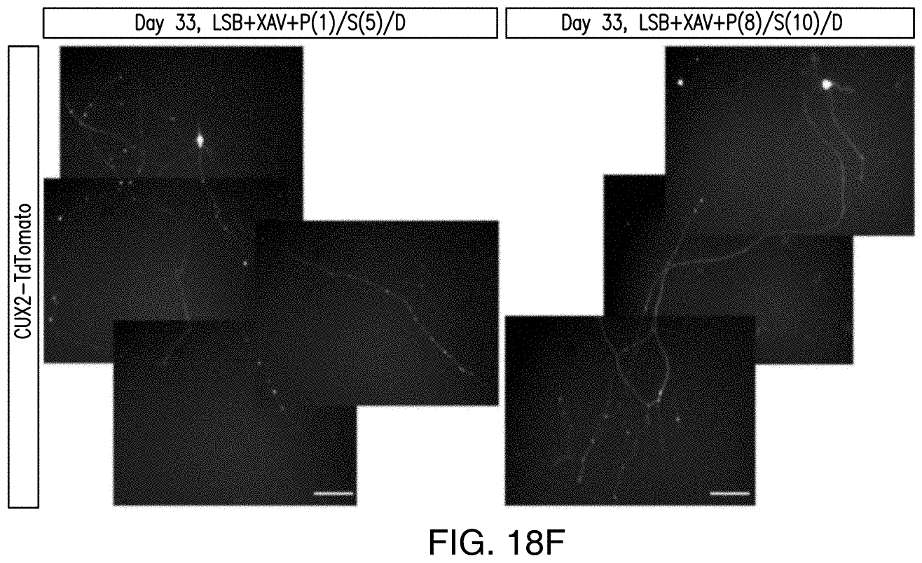

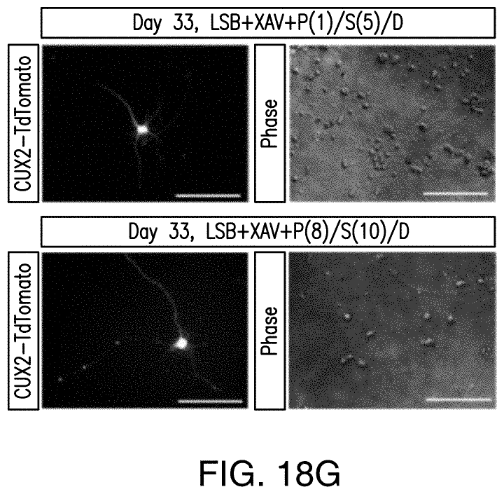

Cortical projection neurons are produced in an inside-out manner¹⁴. Up-regulation of TBR1 and REELIN by day 13 of differentiation suggested a potential bias towards generating the earliest born deep cortical layer neurons. However, further maintenance of P1S5D or P8S10D cultures in the absence of FGF-ERK and Notch inhibition (day 13-55) (FIG. 3F) enabled generation of neurons expressing markers representing a broader range of cortical layers, such as FOXP2 (layer V-VI), CTIP2 (layer V), SATB2 (layer II-III, V), RGS4 (layer II-III, V) (FIG. 3, FIG. 17) as well as generation of upper layer CUX2+(layer II-IV) neurons monitored by using a tamoxifen-inducible CUX2 reporter hESC line (FIG. 18A-D). P1S5D or P8S10D treated cells started to produce CUX2+ post-mitotic neurons with mature morphologies as early as day 33, compared to day 55 using a protocol without acceleration (FIG. 18E-G). While cortical neurogenesis is considerably accelerated, no upregulation of glial markers, such as GFAP, AQP4 or OLIG2 was observed. Similarly, there was no induction of retinal fate markers such as CHX10 (FIG. 17). The quantification of TBR1+, CTIP2+ and SATB2+ neurons (FIG. 3H) suggested that in vitro-derived neurons may follow a temporal order of marker expression consistent with in vivo corticogenesis. To further address the specific timing of neuron subtype derivation in vitro, we performed birth-dating experiments (FIG. 3F). EdU co-labeling with layer-specific markers showed successive waves of cell birth (FIG. 3I-J). Thus, our data demonstrate highly efficient induction of layer VI and indicate the feasibility of accelerated derivation of upper-layer neurons using a modified small-molecule timing regimen.

Many cells express layer VI marker TBR1, FOXP2 and TLE4 by day 33, and start to express layer V marker CTIP2, as well as layer II-V marker SATB2 in more than 10% of the cells, which is the earliest expression compared to current published protocols on cortical neuron induction from hPSCs. As shown in FIG. 3G by day 33 of induction, P1S5D culture gives rise to neurons of deep layers TBR1+ and FOXP2+(layer V-VI), CTIP2+(layer V) and SATB2+ corpus callosum neurons (layer II-V). Also, as FIG. 3G shows, further maintenance of P1S5D or P8S10D cultures in the absence of ERK and Notch inhibition (through day 55 of induction) enabled the efficient production of neurons expressing upper layer markers such as CTIP2+ and SATB2+(approximately 60% and 20% of TUJ1+ neurons, respectively).

The culture continues to enrich for more neurons belonging to the upper layers at day 45 and day 55, as quantified in FIG. 3H. To address the timing of birth for neurons of different layers, the disclosed methods perform EdU pulse labeling (FIGS. 3I and 3J), which demonstrates that early born cell population are the layer VI TBR1+ neurons, which is consistent with the intrinsic temporal mechanism of corticogenesis.

Rapid Induction of Neuronal Function

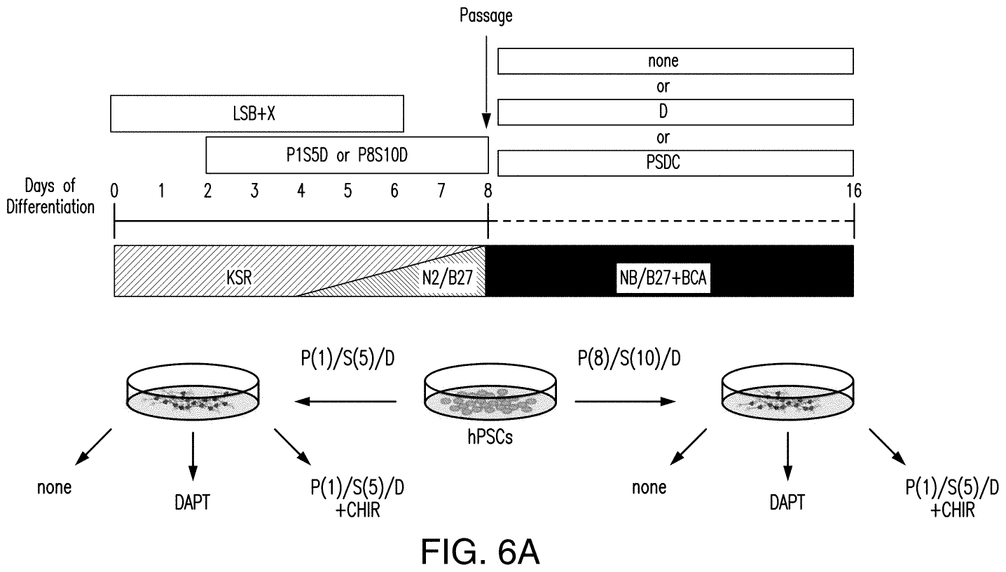

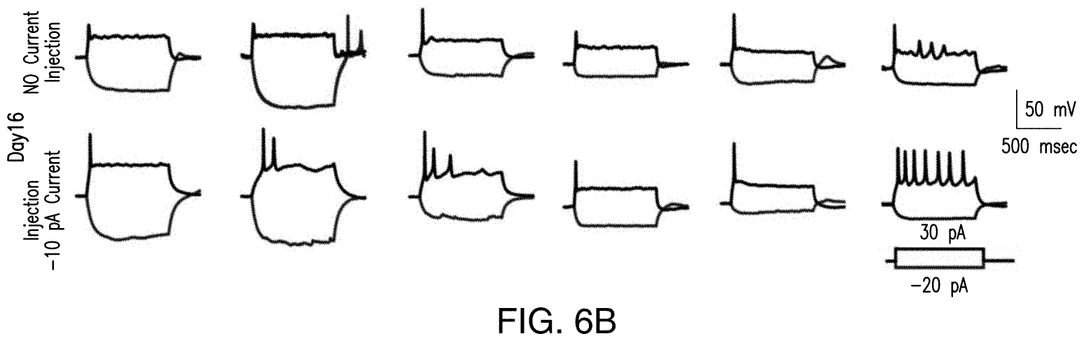

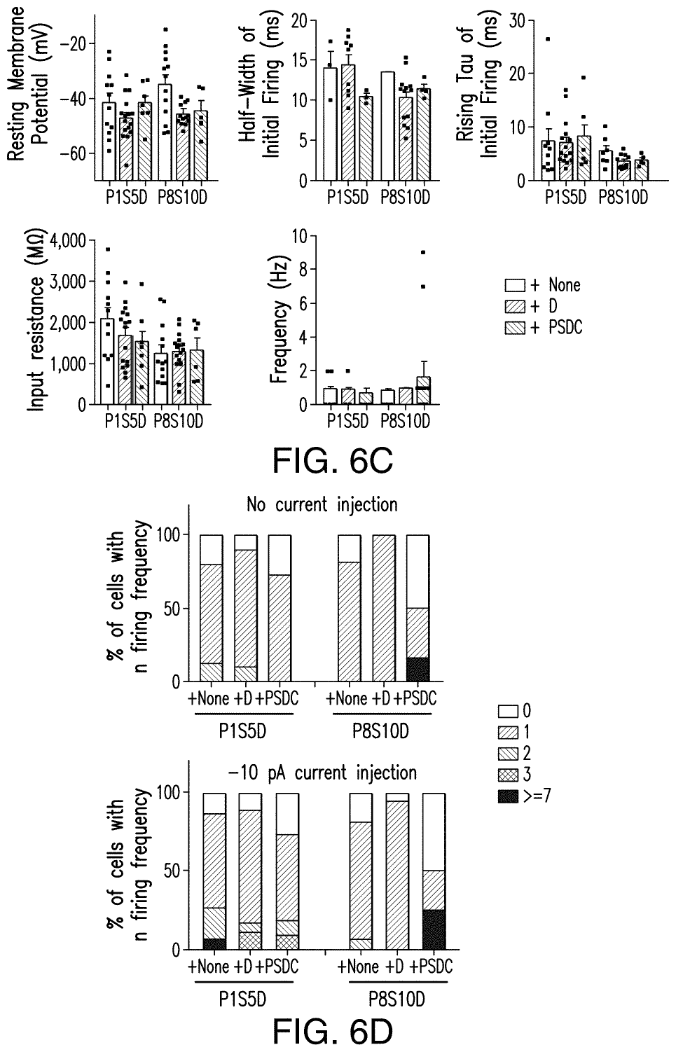

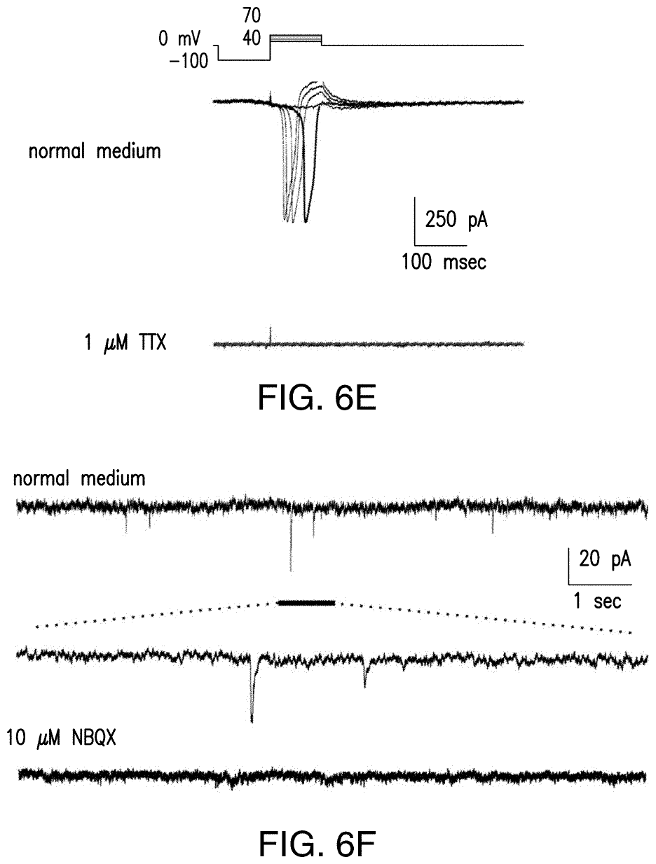

The disclosed methods demonstrate highly efficient induction of TBR1+ cells under rapid CNS neuron induction conditions, but also can indicate the feasibility of deriving neurons expressing upper layer markers using a modified small molecule timing regimen. The disclosed methods can further examine whether rapid induction of neuronal markers is paralleled by rapid in vitro functional maturation such as the ability to spontaneously fire repetitive action potentials. Functional maturation of hPSC-derived neurons has been previously demonstrated¹⁵ with firing of action potentials typically occurring at about 50-100 days of differentiation. To assess maturation, the disclosed methods can culture cells that were induced towards neuronal fate for 8 days under P1S5D or P8S10D conditions followed by an additional 8 days in either i) basal medium without any small molecule inhibitors, ii) addition of DAPT only or iii) addition of DAPT with SU5402, PD and CHIR99021 (CHIR) (P/S/D/C) (FIG. 6A). The GSK30 inhibitor CHIR was included for this final differentiation step as it exerted a strong pro-survival effect on cultures maintained in P/S/D and had been previously shown to promote neuronal differentiation including axonal outgrowth and synapse formation by triggering activation of canonical WNT signalingl^{16, 17}. Remarkably, P8S10D cells maintained under P/S/D/CHIR for 8 days (day 16 of differentiation from pluripotent state) showed mature electrophysiological properties characterized by the firing of trains of action potentials spontaneously at rest membrane potential or upon induced hyperpolarization after −10 pA current injection (FIG. 6B). In all conditions, 70%-80% of the neurons recorded were capable of firing, with ˜20%-30% neurons showed more mature firing patterns (˜train of 10 action potential firing peaks) in P8S10D cells with P/S/D/C (FIG. 6D).

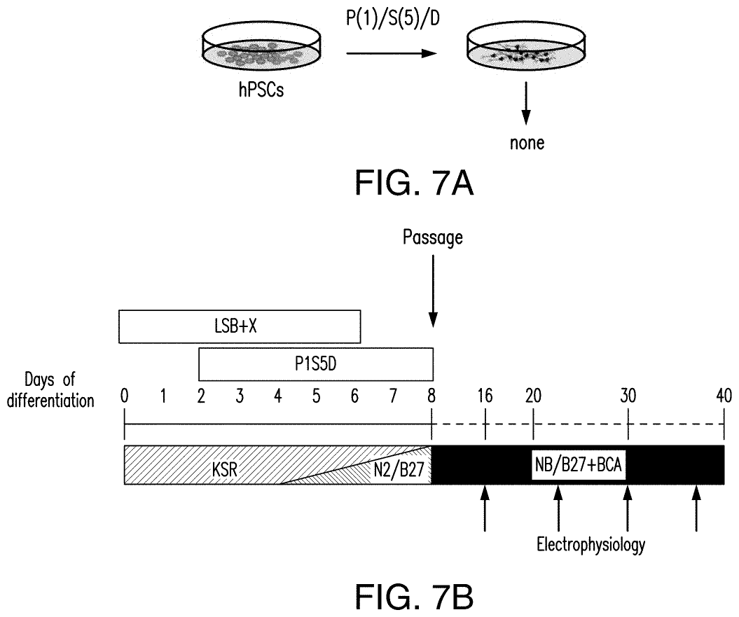

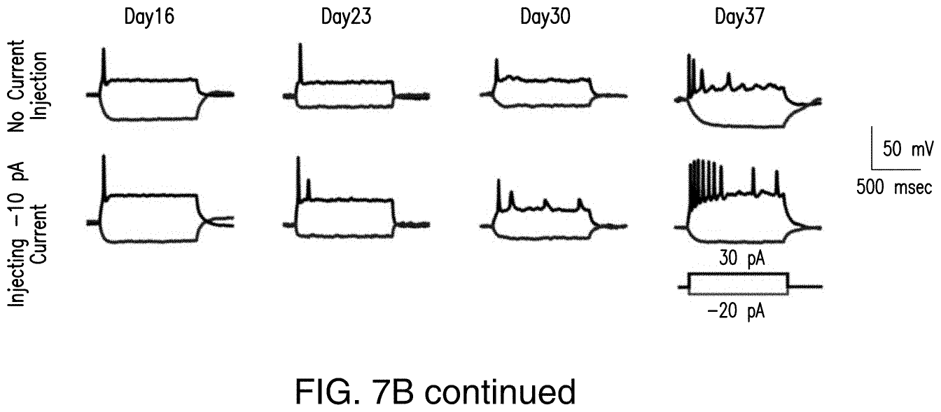

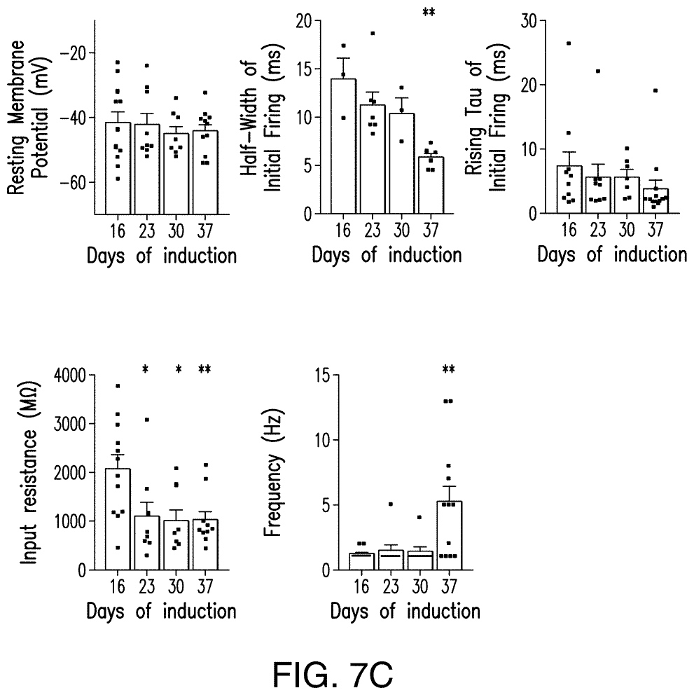

Additional parameters of neuronal maturation assessed in both P1S5D and P8S10D neurons include resting membrane potential, action potential half-width and rise rate (Tau) of initial firing, input resistance and maximum firing frequency (FIGS. 6C and 6D). While maintaining cells in P/S/D/CHIR resulted in the most mature neuronal properties, even the mildest condition (P1S5D treated cells in basal medium without any small molecule inhibitors) resulted in neurons with mature firing patterns by day 37 (FIGS. 7A-7C), a time period considerably faster than in most previous hPSC-derived cortical neuron differentiation protocols.

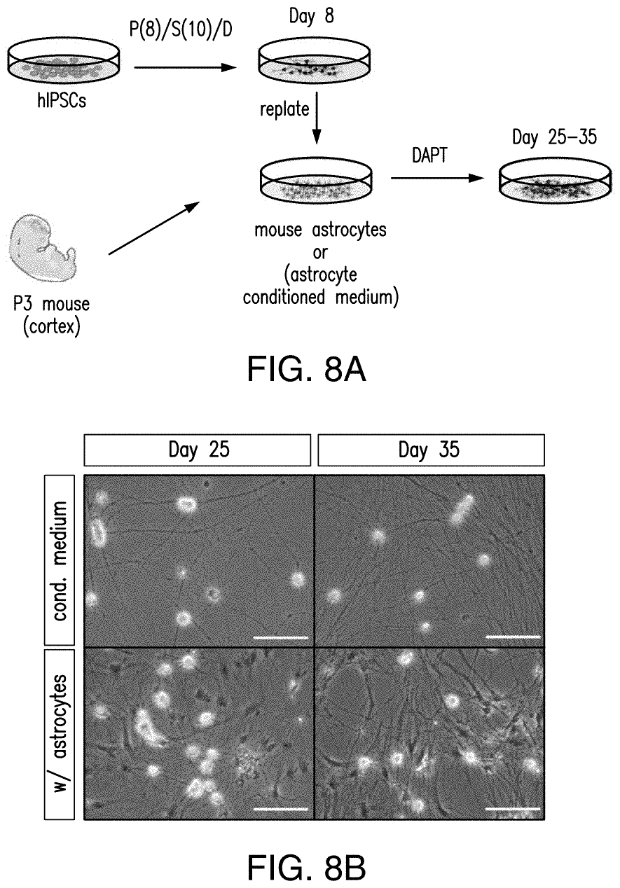

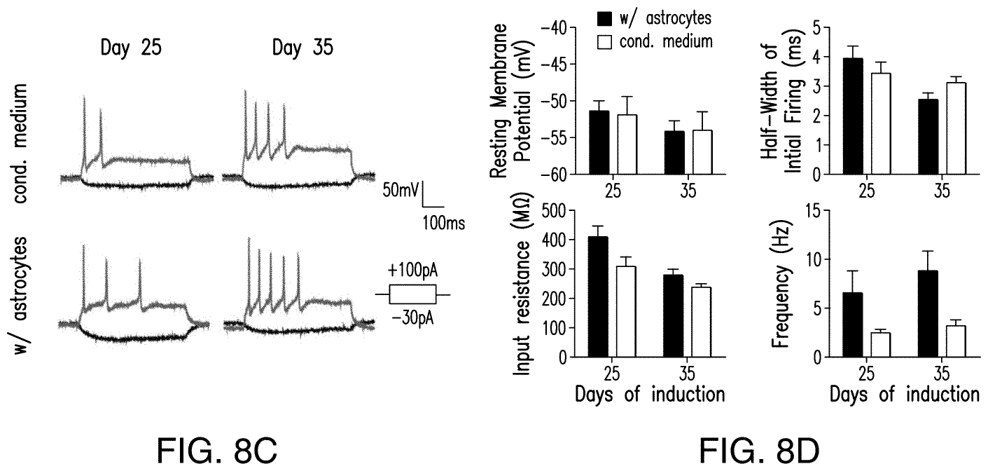

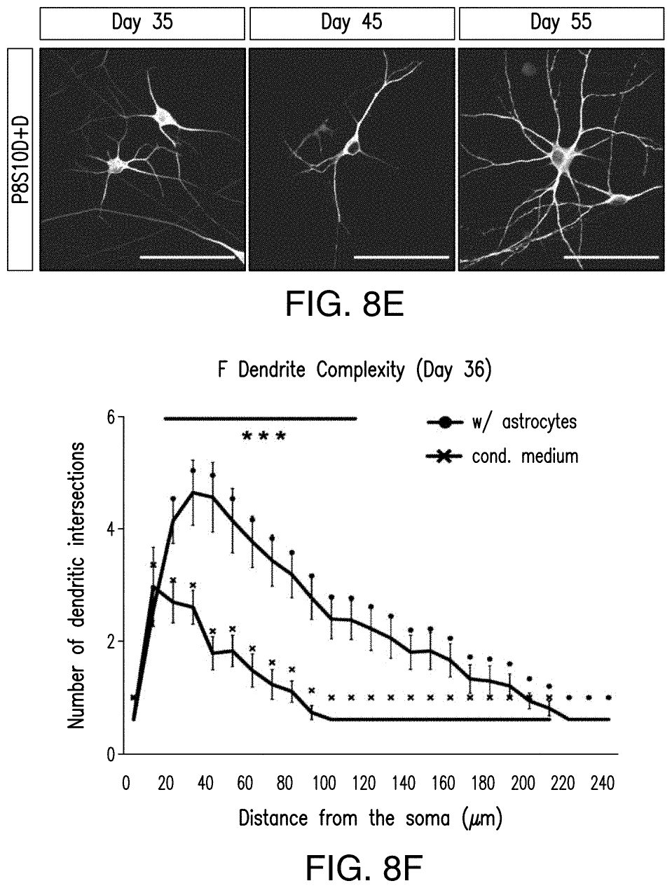

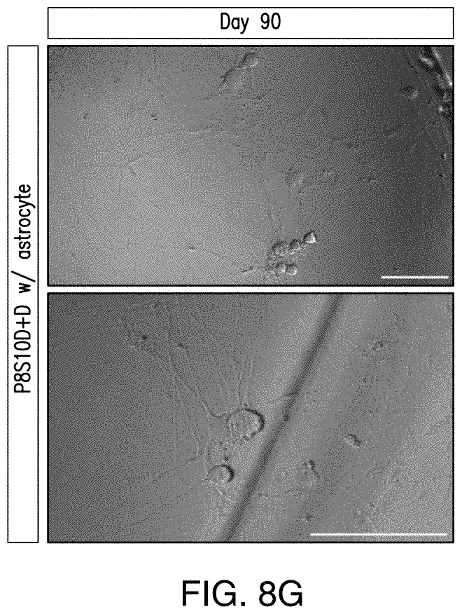

Robust voltage-dependent sodium channel responses were also observed and blocked by Tetrodotoxin (TTX) (FIG. 6E). In addition, cells exhibited spontaneous excitatory postsynaptic currents (sEPSCs) which were inhibited by NBQX, a specific AMPA receptor antagonist (FIG. 6F), indicating the formation of functional excitatory synapses. While functional maturation data were obtained in the absence of any astrocyte co-culture, replating P8S10D cultures on mouse astrocytes (day 8) or culture in astrocyte conditioned medium (FIG. 8A-8C) improved overall neuronal survival and yielded neurons with decreased input resistance and enhanced morphological complexity (FIG. 8D-8F). Additionally, we tested whether the addition of astrocytes would further accelerate the maturation or promote the maintenance of the neurons. Indeed, culturing P8S10D derived neurons on mouse astrocytes or in astrocyte conditioned medium in the presence of DAPT (FIG. 8A-C) improved overall neuronal survival and enabled long-term maintenance of rapidly induced neurons from 70 days to beyond 90 days (FIG. 8G). Cultures on astrocytes further yielded neurons with decreased input resistance and enhanced morphological complexity, indicating increased neuronal maturity (FIG. 8E-G).

In vivo analysis of hPSC-derived neurons using iDISCO-based whole brain analysis

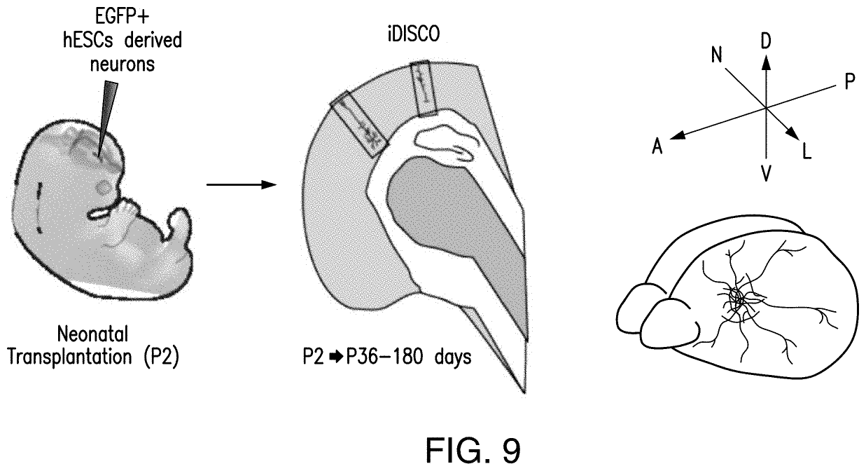

The in vitro data demonstrate that our combinatorial small molecule protocols can rapidly induce neurons with cortical marker expression and functional electrophysiological properties. However, to more fully assess long-term survival, capacity for axonal projections and integration into host circuitry, in vivo transplantation studies were performed. Immature neurons at day 8 of differentiation derived from hESCs constitutively expressing EGFP were grafted into the somatosensory cortex of P2 NOD-SCID IL2Rgc^{-/-} mice (FIG. 9).

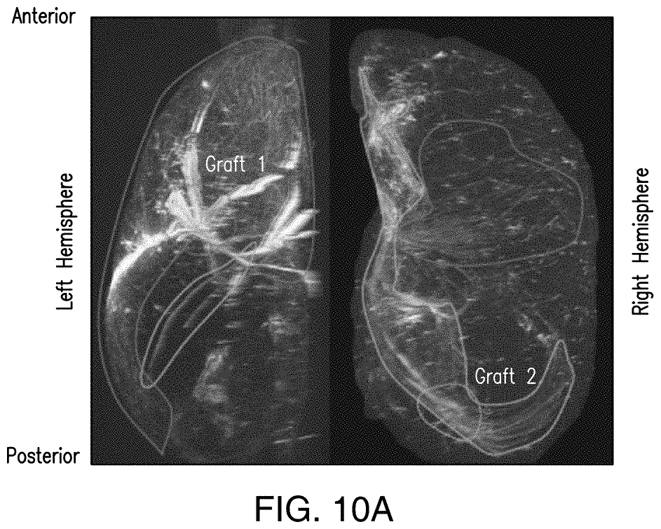

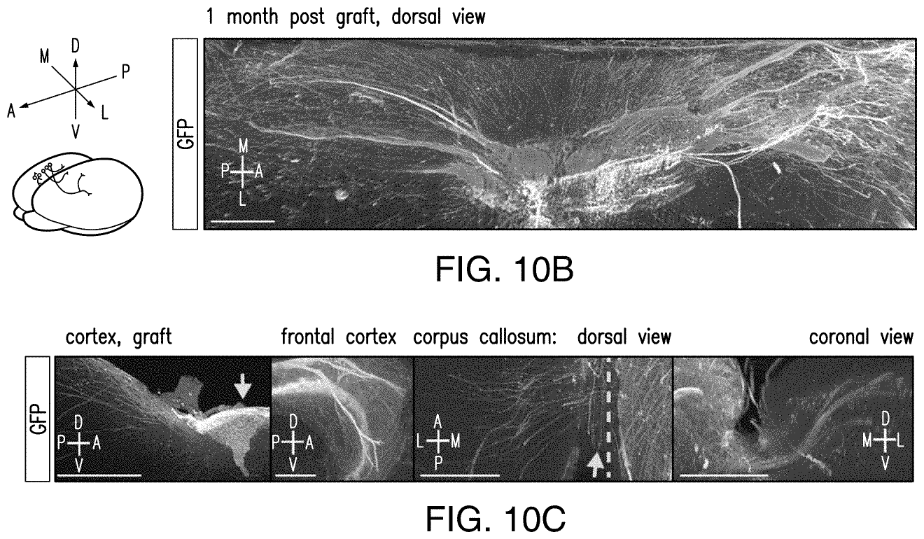

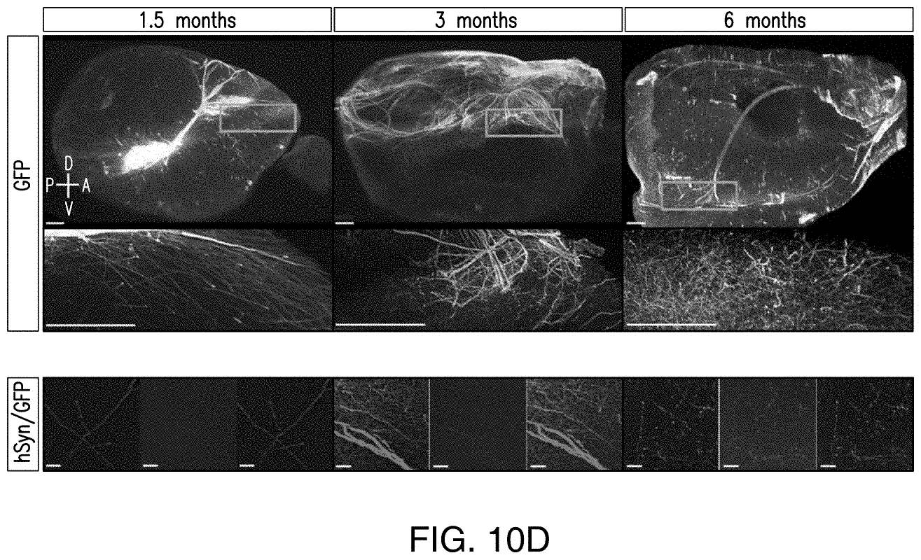

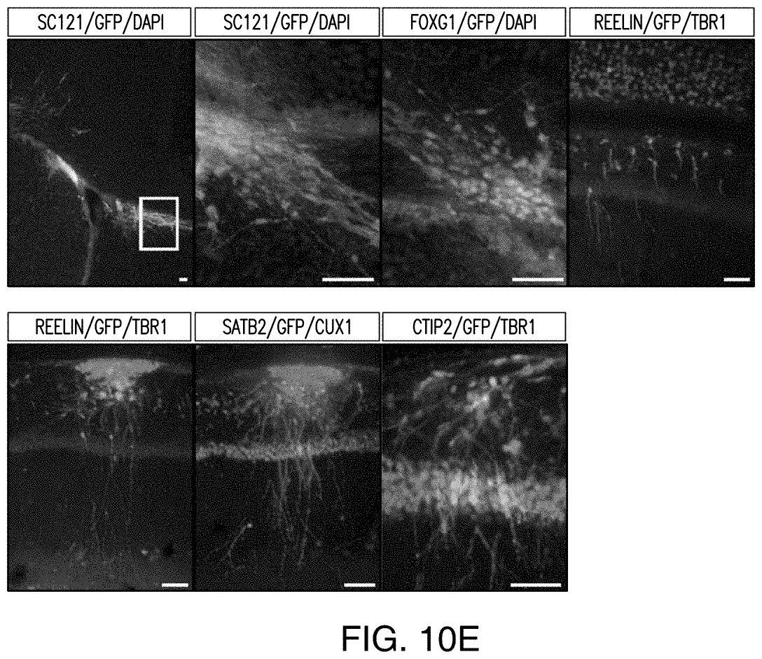

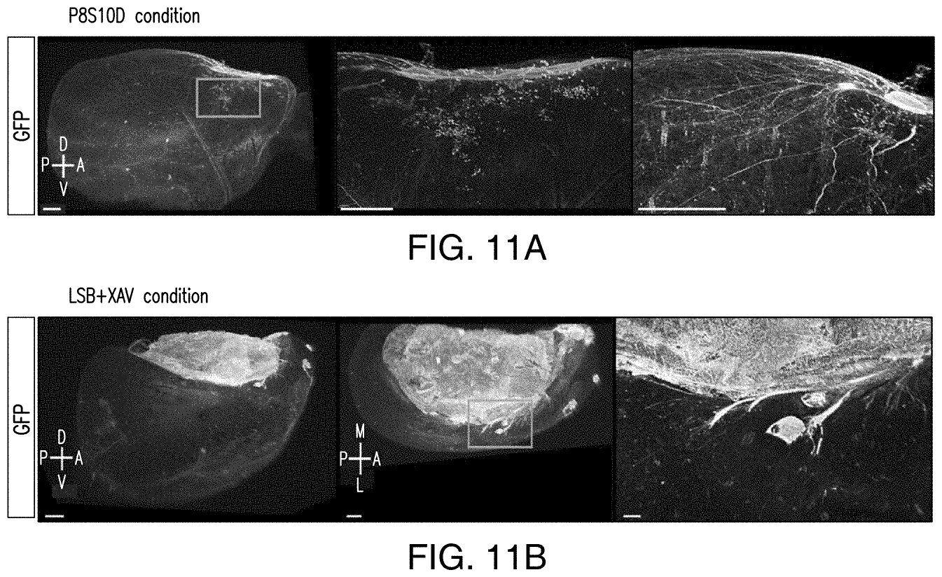

Brains of the grafted animals were collected at 1-6 months after grafting and subjected to whole brain immunofluorescence imaging following the iDISCO¹⁸ clearing and whole mount immunohistochemistry protocol (FIG. 10A). Most transplantation studies were carried out using the P1S5D paradigm which showed robust in vivo survival up to 6 months after transplantation, the latest time point tested in our study. P8S10D neurons showed more variable in vivo survival with evidence of engraftment and axonal projections in only a subset of the animals at 1 month after transplantation (FIG. 11A). Matched day 8 cells from the LSB+X condition showed extensive graft overgrowth with minimal evidence of neuronal differentiation or graft integration (FIG. 11B). These data are reminiscent of previous results suggesting that early neuroepithelial, ‘rosette-stage’ cells result in tumor-like overgrowth²². Therefore, differentiation of neuroepithelial cells toward later-stage neural precursors or neurons is critical in reducing the risk of neural overgrowth

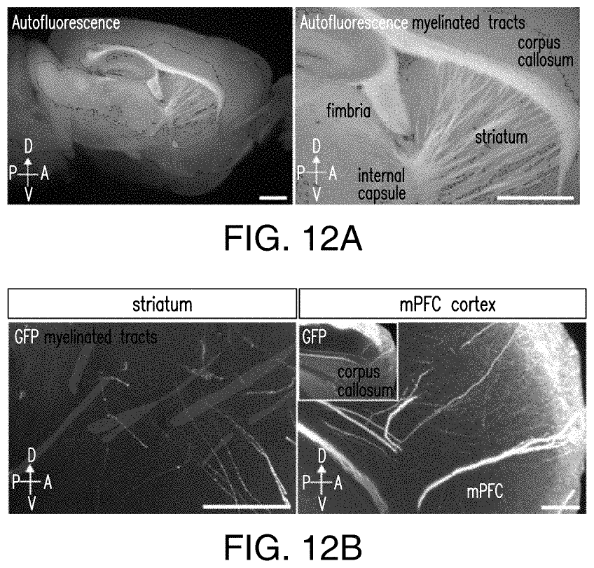

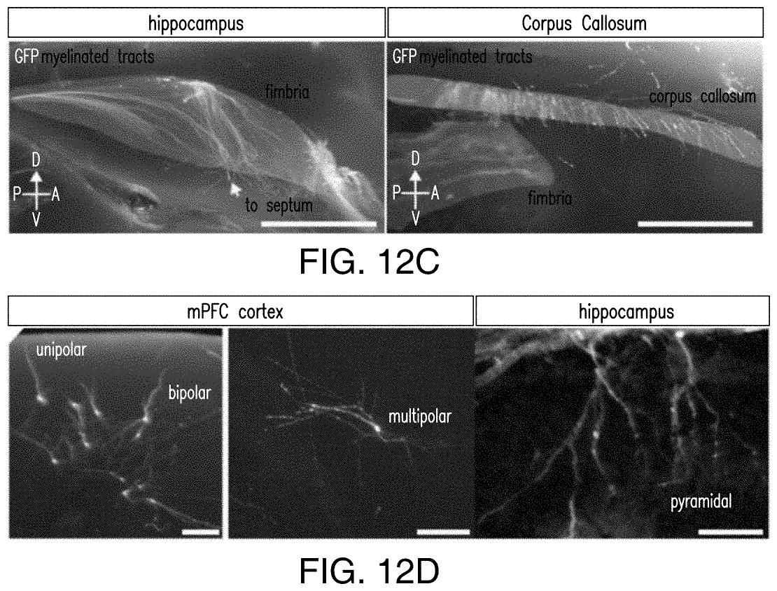

Detailed analysis of brains grafted with P1S5D neurons at 1 and 1.5 months after grafting allowed visualization of the graft core and neuronal projections (FIG. 10C). After 1 month, GFP+ grafted cells developed extensive defasciculated projections across all cortical layers (FIG. 10B). A few long dense bundles were also consistently seen in cortical layer VI. Most of the projections terminated in the pre-frontal motor cortex and frontal cortex, although many axons were also traced in the ipsilateral hippocampus and contralateral cortex through the corpus callosum (FIG. 10C). Very sparse graft-derived fibers were observed in the striatum suggesting that grafted neurons preferred projecting across cortical regions rather than targeting subpallial regions. Using autofluorescence to map host axonal pathways (FIG. 12A), we observed that the majority of graft-derived fiber bundles followed endogenous tracts (FIG. 12C). However, some fibers projected outside of the host descending tracts (FIG. 12B).

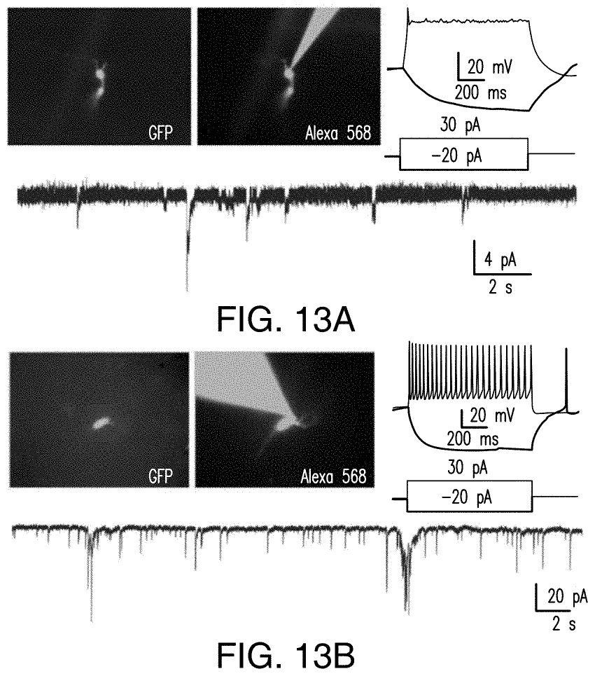

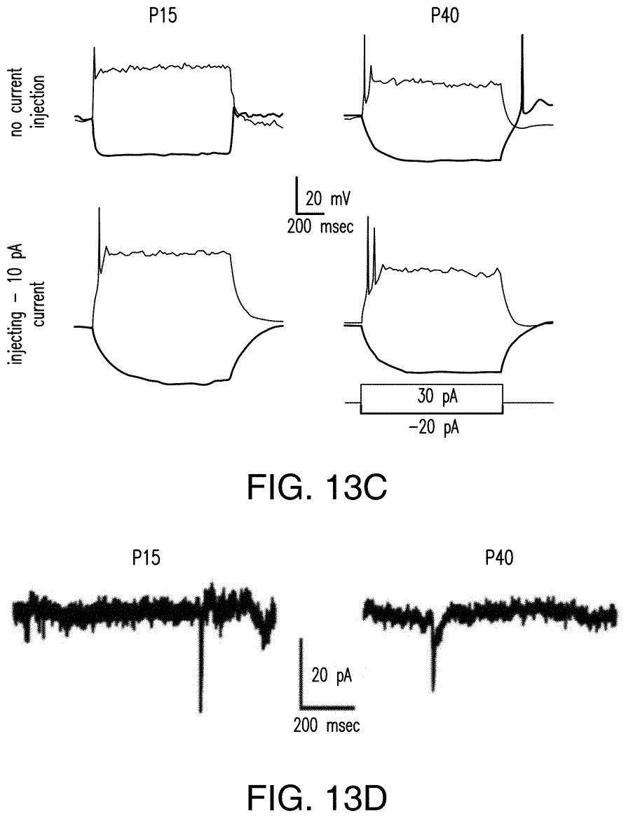

By 1.5 months after transplantation the majority of graft-derived axonal fiber bundles followed a straight trajectory and an enlarged terminal structure reminiscent of growth cones, a pattern characteristic of ongoing path finding with only limited terminal arborization (FIG. 10D, left panel). In contrast, by 3 months after transplantation and most pronounced at 6 months (FIG. 10D, middle and right panels), there was extensive terminal arborization of human axons in matched target areas (FIG. 10D, middle versus left panel). Concomitant to extensive arborization, a dense network of human synaptophysin positive structures were observed, which were co-localized with the GFP+ fibers in several target areas such as the host hippocampus (FIG. 10D, bottom right panel). The grafted neurons exhibited a range of morphologies, with unipolar, bipolar, multipolar and pyramidal shapes (FIG. 12D). iDISCO based analysis was complemented with conventional immunohistochemical analyses that confirmed in vivo cortical marker expression in human cells including expression of the general forebrain marker FOXG1 and layer specific markers such as REELIN, SATB2 and CTIP2 (FIG. 10E). In addition, preliminary evidence of in vivo function by electrophysiology were obtained (FIG. 13). Expression of SATB2+ in the grafted neurons is compatible with commissural neuron identity matching the presence of commissural axonal projections in the iDISCO-based studies (FIG. 10D). The data indicated that P1S5D induced neural cells at day 8 of differentiation were capable of in vivo survival and extensive axonal projections within the cortex. While P8S10D induced neurons showed overall reduced graft size and viability, animals with surviving grafts showed extensive fiber outgrowth and arborization already at 1.5 months.

### DISCUSSION

The disclosed methods can indicate that P1S5D induced neural cells at day 8 of differentiation are capable of in vivo survival and extensive axonal projections within the cortex. While P8S10D neurons showed overall reduced graft size and viability, animals with surviving grafts showed extensive fiber outgrowth and arborization at 1.5 months. Future more detailed studies will be required to determine whether P8S10D grafts undergo more rapid in vivo maturation as compared to P1S5D cells. Interestingly, matched day 8 grafts from dual SMAD inhibition cultures (LSB+XAV) in the absence of any acceleration showed extensive graft overgrowth (FIG. 11B) with minimal evidence of neuronal differentiation. These data match previous results from our lab and others suggesting that early neuroepithelial “rosette-stage” cells result in tumor-like overgrowth of neuroepithelial cells¹⁹. The grafted cells also show heterogeneity in axon path finding, as some neurons extend axons following the intrinsic white matters, some neurons extend axons towards random directions (FIG. 12). Therefore, further differentiation towards later stage neural precursor or neuronal lineages is critical in reducing the risk for neural overgrowth.

The disclosed methods represent a first application of iDISCO for mapping hPSC-derived graft survival, axonal projections and host innervation. The iDISCO data include whole brain immunohistochemistry and imaging for GFP as well as for expression of human specific markers such as human synaptophysin. However, the technology should be suitable for use with most any human specific markers to monitor specific aspects of graft biology. In future studies it may be particularly interesting to apply iDISCO to mapping region-specific projections of defined hPSC-derived cortical lineages such cells with selective cortical area and layer identity²⁰. The assay could also serve as a tool to define neurons of related lineages but distinct projection patterns such as midbrain dopamine neurons of A9 (substantia nigra) versus A10 (ventral tegmental area) identity and to map terminal projection patterns of neurons placed at heterotopic²¹ versus orthotopic locations²².

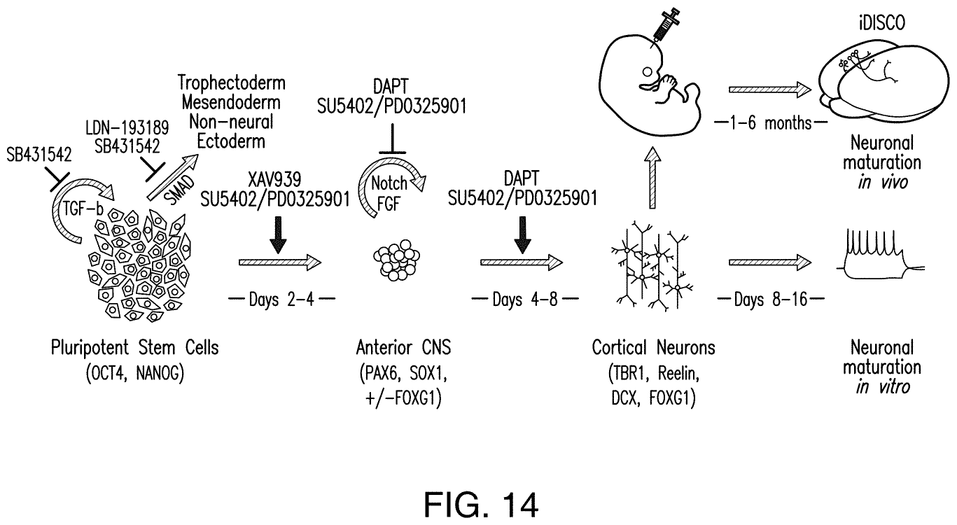

The disclosed methods provide a rapid induction protocol that can yield cortical neurons with mature electrophysiological properties by day 16 of differentiation and capable of in vivo engraftment and long-distance projections in postnatal mouse cortex (FIG. 14). According to embodiments of the invention, similar acceleration strategies can be developed for other neuron subtypes following the example of sensory neuron induction⁷ and now cortical neuron derivation. Such rapid directed differentiation protocols may considerably reduce time and cost for obtaining specific neurons relevant for disease modeling, drug discovery or cell therapy. The cortical neurons derived under the current P1S5D or P8S10D conditions are biased toward deeper cortical layers.

The time frame of generating functional neurons in ˜2 weeks of differentiation is comparable to the speed achieved when using transcription factor based neuronal induction of hPSCs such as NGN2-based differentiations²³. However, directed differentiation via small molecules may offer greater flexibility for generating specific neurons and obviates the need for genetic modification. Finally, with the increasing ability to recreate human lineage diversity from hPSCs in vitro, this study is a step towards modulating timing of differentiation and maturation as independent parameters on the road towards harnessing the full potential of iPSC-derived neurons in disease modeling and regenerative medicine.

### Methods

hESC Lines and hiPSC Line Generation

hESCs (WA-09, passages 32-60) were obtained from WiCell and maintained up to passage 60. The hESC SOX10::GFP bacterial artificial chromosome reporter line (WA-09; passage 40-70) was generated as reported previously⁷. Constitutive EGFP+ hESC line (WA-09; passage 35-60) was generated as reported²⁴. For hiPSC induction, fibroblasts were prepared by digesting skin punch biopsies following a protocol generously shared by Michael Sheldon (Rutgers University). Briefly, skin punches were digested in a mixture of collagenase (1%) and Dispase (1 unit/ml) in DMEM+10% FBS for 16-18 hrs at 37° C. in a tissue culture incubator. After digestion, the epidermal layer was discarded and the partially digested dermal layer was quartered onto the surface of a dry tissue culture dish and was left undisturbed for 2-5 min to encourage adhesion to the dish. DMEM+10% FBS was carefully added to the well so as not to detach the dermal layer. Cultures were fed every 3 days until confluent foci covered around ⅔ of the well. Once confluent, cultures were passaged by trypsinization and expanded for 4-5 passages before reprogramming. Induced pluripotent stem cells were made using the original CytoTune iPS Reprogramming Kit (Life Technologies, A1378002) using the manufacturer's protocol with a few modifications. Human ES medium containing 1 mM Valproic acid (EMD Millipore) was added from day 2-9. After 2-3 weeks, individual iPS clones were picked and propagated as iPS lines. To verify that each of the three iPS subclones from a given individual were truly non-clonal, colonies from 3 different wells that derived from 3 separate transductions were selected. Each line was propagated for 10 passages before performing quality control assays. Expression of OCT4, NANOG, SSEA-3, SSEA-4 and Tra-1-81 was confirmed before. Clones that expressed all pluripotency markers were verified to have a normal karyotype by the Molecular Cytogenetics Core Facility at MSKCC. The amount of Sendai Vector present after 10 passages was quantitated using the TaqMan iPSC Sendai Detection Kit (Life Technologies A13640) and only clones with less than 0.01% Sendai virus amplicon (Mr04269880_mr) were used.

Generation of PAX6::H2B-GFP and SIX1::H2B-GFP Lines (Passage 40-65)

The PAX6-P2A-H2B-GFP and SIX1-P2A-H2B-GFP donor constructs were generated by performing In-Fusion cloning (Clontech) into the pUC19 backbone. Homology arms were generated by using genomic DNA, H2B:GFP was a gift from Geoff Wahl (Addgene, plasmid #11680), Pgk-Puro was amplified from the AAVS1 hPgk-PuroR-pA donor plasmid (a gift from Rudolf Jaenisch (Addgene, plasmid #22072)). TALE nucleases were generated using the TALE-Toolbox provided by Addgene²⁵. Sequences targeting the stop codon of Pa×6 were: TGTCCTGTATTGTACCACT (SEQ ID NO: 1) and TGTATACAAAGGTCCTTGT (SEQ ID NO: 2), for Si×1 were: TCTCTGCTCGGCCCCCTCA (SEQ ID NO: 3) and TTGGGGTCCTAAGTGGGGA (SEQ ID NO: 4). Briefly, 25 μg of donor plasmid and 5 μg of each TALEN were nucleofected into 10×106 H9 hESCs. Puromycin selection was applied 72 hrs after nucleofection to isolate resistant clones. Clones were amplified and genomic PCRs confirming targeting were performed. All positive clones used had normal karyotype.

Generation of Transgenic CUX2 Conditional Reporter Line

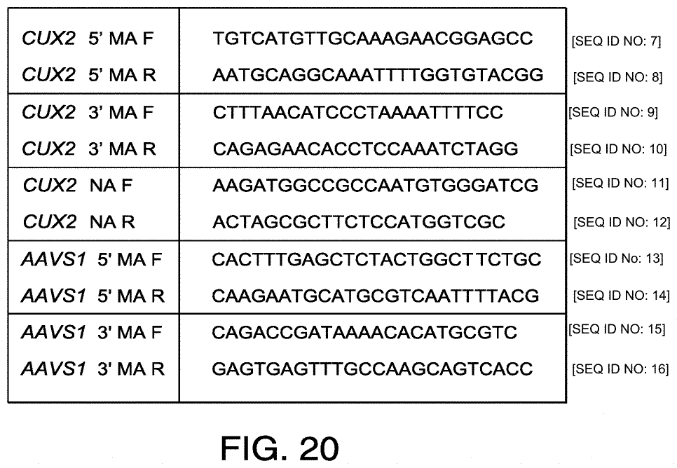

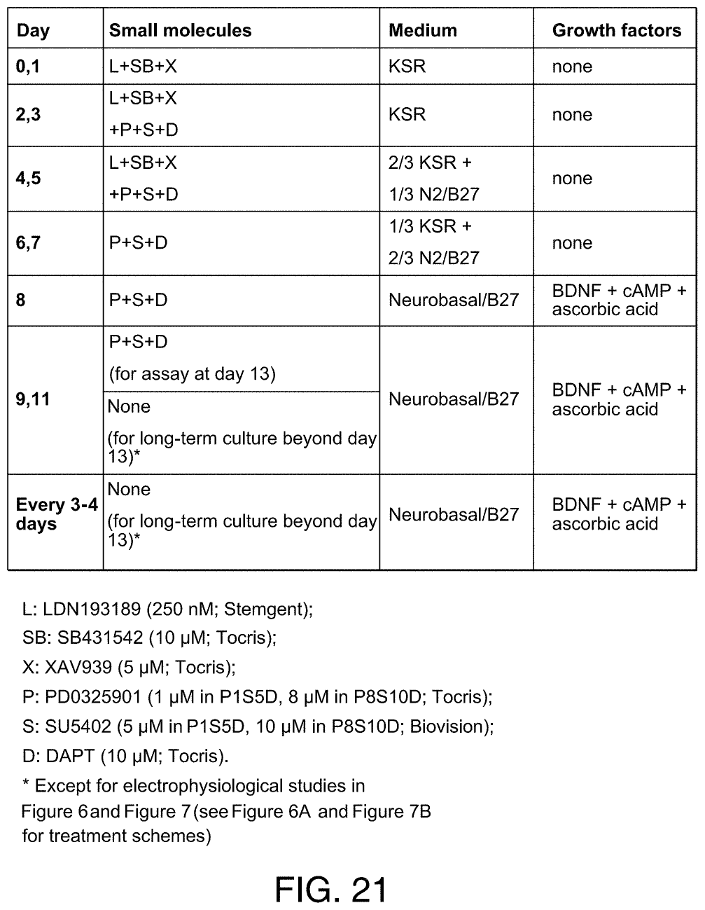

The CUX2::CreER^{T2}/AAVS1-CAG::FLEX/tdTomato line was created in the RUES2 background by two sequential nucleofection and selection cycles. In the first round, 2 μg of CUX2::CreER^{T2}/FRT-Puro-FRT-TK homology donor was electroporated into 2×10⁶ early passage hESCs together with TALENs targeting the CUX2 initiation codon (FIG. 18). Nucleofection was carried out using Amaxa nucleofector solution L (Lonza). Single cells were obtained by treating cultures with Accutase (Innovative Cell Technology), and cells were maintained in the ROCK-inhibitor Y-27632 (10 μM; Tocris) after nucleofection for 3 days. Nucleofected cells were subsequently grown for 2 weeks in puromycin selection medium maintained for the initial 10 days. Ganciclovir (2 μM) was also added for negative selection of random integrations. After 2 weeks, 22 clones were selected for further characterization by PCR genotyping, sequencing, and karyotyping. One clone, which satisfied all criteria, was expanded and subjected to a second round of nucleofection with 2 μg of AAVS1 CAG::FLEX tdTomato/BSD homology donor, 0.5 μg each of AAVS1 right and left TALENs (Addgene), and 2 μg pCAG-Flpe (Addgene). The Flp recombinase was added to excise the FRT-Puro-FRT cassette from the transgene in the CUX2 locus. The nucleofected cells were then grown for 2 weeks in blasticidin selection. 12 clones were subsequently expanded for PCR genotyping and confirmed for excision of the FRT-Puro-FRT cassette. Out of the clones that were found to carry the transgene, one clone was karyotyped and chosen for further experiments. A list of primers used for genotyping is provided in FIG. 20.

Culture of Undifferentiated Cells and Neuronal Induction (Day 0-13 of Differentiation)

hPSC lines were maintained with mouse embryonic fibroblasts (MEFs; Globalstem) pre-plated at 16,000 cells/cm² on gelatin-coated tissue culture plate. Medium contained DMEM/F12, 20% (v/v) Knockout Serum Replacement, 1 mM L-glutamine, 100 μM MEM nonessential amino acids and 0.1 mM β-mercaptoethanol (Life Technologies). 10 ng/ml FGF2 (R&D Systems) was added after sterile filtration. Cells were fed daily and passaged weekly using 6 U/ml dispase. For neural differentiation, cells were disassociated with Accutase and pre-plated as reported¹ at the density of 200,000 cells/cm² supplemented with 10 μM Y-27632 on matrigel coated plates, and started differentiation the next day when confluent. KSR medium which contained 820 ml of Knockout DMEM, 150 ml Knockout Serum Replacement, 1 mM L-glutamine, 100 μM MEM nonessential amino acids and 0.1 mM β-mercaptoethanol was used to start differentiation. Inhibitors used in LSB+X/P/S/D induction included LDN193189 (250 nM; Stemgent), SB431542 (10 μM; Tocris), XAV939 (5 μM; Tocris), PD0325901 (1 μM in P1S5D, 8 μM in P8S10D; Tocris), SU5402 (5 μM in P1S5D, 10 μM in P8S10D; Biovision), DAPT (10 μM; Tocris). More inhibitors used in other induction described in the paper include CHIR99021 (6 μM in LSBC, 3 μM in LSB+C/S/D; Stemgent). N2 medium¹ with B27 supplement (N2/B27; Life Technologies) was added in increasing ⅓ increment every other day from day 4, until reaching 100% neurobasal/B27/L-Glu containing medium (NB/B27; Life Technologies) supplemented with BDNF (20 ng/ml; R&D), dibutyryl cAMP (0.5 mM; Sigma-Aldrich) and ascorbic acid (0.2 mM; Sigma-Aldrich) (BCA) at day 8. An outline of the P1S5D and P8S10D differentiation scheme (day 0-13 of differentiation) is presented in FIG. 2A, with detailed daily feeding instructions shown in FIG. 21 and as described below in the step-by-step protocol of P1S5D and P8S10D induction from day 0-13, and long-term culture beyond day 13. We tested 3 different lots of KSR which gave consistent results in neuronal yield by day 13.

Step-by-Step Protocol of P1S5D and P8S10D Induction from Day 0-13, and Long-Term Culture Beyond Day 13
1. Coat the tissue culture dish with matrigel (354234; BD): dilute 1:30 in DMEM/F₁₂ and apply to the tissue culture dish. Leave at room temperature for 2 hrs.
2. Detach cells: wash the cell culture once with PBS, disassociate with Accutase at 37° C. for 0.5-1 hr until most cells are detached.
3. After wash, resuspend cells in hESC medium supplemented with Y-27632 (10 μM), and plate on gelatin-coated plate at 37° C. for 1 hr to remove MEFs.
4. Collect cell suspension. After washing, plate cells in MEFs conditioned medium (conditioned hESC medium collected from fully confluent MEFs culture) supplemented with Y-27632 and FGF2 (10 ng/ml) at 200,000 cells/cm².
5. Start differentiation the next day (day 0). KSR medium which contained 820 ml of Knockout DMEM, 150 ml Knockout Serum Replacement, 1 mM L-glutamine, 100 μM MEM nonessential amino acids and 0.1 mM β-mercaptoethanol (Life Technologies) was used to start differentiation. Inhibitors used in P1S5D and P8S10D induction include LDN193189 (250 nM; Stemgent), SB431542 (10 μM; Tocris), XAV939 (5 μM; Tocris), PD0325901 (1 μM in P1S5D, 8 μM in P8S10D; Tocris), SU5402 (5 μM in P1S5D, 10 μM in P8S10D; Biovision), DAPT (10 μM; Tocris). LSB+X were added from day 0-6, and P/S/D were added from day 2-13.
6. N2 medium¹ with B27 supplement (N2/B27; Life Technologies) was added in increasing ⅓ increments every other day from day 4: ⅓ N2/B27 for day 4 and 5, ⅔ N2/B27 for day 6 and 7. Starting from day 8, medium is switched to neurobasal supplemented with B27 (NB/B27), BDNF (20 ng/ml; R&D), cAMP (0.5 mM; Sigma-Aldrich) and ascorbic acid (0.2 mM; Sigma-Aldrich) (BCA). An outline of the P1S5D and P8S10D differentiation scheme (day 0-13) is presented in FIG. 2A.
7. For long-term culture beyond day 13 for the generation of deep and upper layer neurons, cells were pre-plated and differentiated as described in step 1-6 till day 8, then passaged onto PO/laminin/fibronectin coated dishes. These dishes were treated by polyornithine (PO; 15 μg/ml; Sigma-Aldrich) diluted in PBS for 24 hrs at 37° C. After washing with PBS, the dishes were further treated with mouse laminin I (1 μg/ml; R&D system) and fibronectin (2 μg/ml; Sigma-Aldrich) diluted in PBS for 12 hrs at 37° C. Laminin and fibronectin were removed immediately before use.
8. Day 8 cells were dissociated with Accutase at 37° C. for 0.5-1 hr. After washing, the cells were plated onto PO/laminin/fibronectin coated dishes at 150,000 cells/cm² (P1S5D group) or 300,000 cells/cm² (P8S10D group), respectively, in NB/B27+BCA.
9. Medium is changed every 3-4 days and 1 μg/ml laminin was added weekly for better attachment of neurons.
10. The cells were then assessed at various in vitro time points for electrophysiological recordings, immunohistochemistry, and RNA extraction.
11. For a summary of daily feeding instructions, see FIG. 21.

Rapid neuronal differentiation in ESSENTIAL 6™ medium (E6)

The hPSC line (WA-09) was maintained in vitronectin (VTN-N; ThermoFisher Scientific) coated culture plates in ESSENTIAL 8™ medium (with supplement E8). Cells were fed daily and passaged every 5 days with EDTA solution. For neural induction, cells were dissociated and pre-plated in E8 the same way as described for KSR based induction. Differentiation was started the next day when cells were confluent. Inhibitors used in LSB+X induction in E6 included LDN193189 (100 nM) and SB431542 (10 μM) for treatment of 10 days, and XAV939 (2 μM) for treatment of 3 days. E6 was used for the initial 10 days, and was switched to N2/B27 starting at day 10. For accelerated induction, cells were treated with LSB+X at concentration above in E6 from day 0 for 3 days. Then starting from day 3, LDN193189 (50 nM), SB431542 (5 μM), XAV939 (1 μM), PD0325901 (0.4 μM), SU5402 (2 μM) and DAPT (5 μM) were added into E6. N2/B27 medium was added to E6 at ⅓ (v/v) from day 5, with ⅓ increment every other day. Inhibitors in N2/B27 include LSB+X+P/S/D at the same concentration as P1S5D in KSR/N2 based induction. LSB+X were withdrawn from day 7 while P/S/D remain. 100% NB/B27+BCA was used from day 9. Inhibitors used in NB/B27 include PD0325901 (1 μM), SU5402 (5 μM) and DAPT (10 μM). An outline of the accelerated differentiation scheme in E6 (day 0-13 of differentiation) is presented in FIG. 3K.

Long-Term Culture Beyond Day 13 for Generation of Deep and Upper Layer Neurons

The long-term culture protocol for the generation of deep and upper layer cortical neurons is schematically illustrated in FIG. 3F, with detailed daily feeding instructions presented in FIG. 21. hPSCs were induced by P1S5D or P8S10D from day 0 as described in FIG. 2A, and passaged on day 8 of differentiation by Accutase-mediated dissociation for 0.5-1 hrs at 37° C. Cells were replated at 150,000 cells/cm² or 300,000 cells/cm² for P1S5D or P8S10D groups respectively onto pre-coated culture dishes. For pre-coating, dishes were exposed to polyornithine (PO; 15 μg/ml; Sigma-Aldrich) diluted in PBS for 24 hrs at 37° C.; after washing with PBS for three times, the culture dishes were further treated with mouse laminin I (1 μg/ml; R&D system) and fibronectin (2 μg/ml; Sigma-Aldrich) diluted in PBS for 12 hrs at 37° C. Laminin and fibronectin were removed immediately before use. Medium used for both passaging and long-term culture was NB/B27+BCA as described above. Medium was changed every 3-4 days and 1 μg/ml laminin was added weekly for maintaining attachment of neurons. The cells were then assessed at various in vitro time points for electrophysiological recordings, immunocytochemistry, and RNA extraction. For results shown in FIG. 8, passaged P8S10D cells were co-cultured with mouse astrocytes or in the presence of astrocyte conditioned medium. The isolation and maintenance of astrocytes for those studies was carried out as described previously³¹. For conditioned medium collecting, astrocytes were fed with NB/B27+BCA, and conditioned medium was collected every 2-3 days. The conditioned medium was then filtered through 0.22 μm membrane pore vacuum filter (Corning) to get rid of cell contamination.

EdU Labeling and Quantification of Cells

EdU was added to the cultures at 5 μM for a window of 48 hrs each starting at various time points of differentiation (day 8, 13, 18, 23, 28, 33), and the cells were fixed at day 40 with 4% paraformaldehyde for 20 mins. EdU was detected with the Click-iT EdU Imaging Kit (Invitrogen) according to the specifications of the manufacturer. Quantification of EdU positive and cortical layer marker positive neurons in the EdU labeling experiments, and the quantification of marker positive neurons and total cells in the long-term culture was carried out using ImageJ with ITCN plugin for nuclei quantification, combined with manual counting. 6 uniform randomly selected image frames from 2 independent batches of cell cultures were captured using a 20× objective and used for quantification. Areas containing clusters could not be properly resolved for co-labeling analysis (EdU, cortical layer markers) were avoided. Quantification of pH3 and cleaved caspase 3 positive cells was also carried out using ImageJ with ITCN plugin. Per culture plate, 4 uniform randomly selected image frames were captured with 10×objective and used for quantification from 2 independent batches of cell cultures. All quantification results were plotted in Prism (version 6.0, GraphPad).

RNA Extraction and qRT-PCR

Cells were lysed with Trizol Reagent (Life Technology) and stored in −20° C. Total RNA was extracted using phenol/chloroform and isopropanol precipitation, and dissolved in ddH2O. cDNA was made using the QuantiTech Reverse Transcription Kit (Qiagen). Quantitative RT-PCR was performed using the Mastercycler Realple×2 (Eppendorf), and GAPDH was used as the housekeeping gene control for normalization. Delta Delta Ct and fold changes were calculated and results were plotted in Prism (version 6.0, GraphPad).

Immunocytochemistry

Cells were fixed with 4% (v/v) paraformaldehyde for 20 min, washed with PBS, permeabilized and blocked using 0.3% (v/v) Triton X-100 in PBS with 1% (w/v) BSA for 1 hr. For immunocytochemistry, cells were incubated with primary antibodies diluted in the same blocking buffer at 4° C. overnight. A list of the primary antibodies used in this study is provided as Table 1. Following several washes, cells were incubated with appropriate AlexaFluor secondary antibodies (1:500; Molecular Probes) and DAPI (1:1000; Thermo Fisher) diluted in the blocking buffer for 1 hr at room temperature. After washing, cells were taken images by Olympus IX71 microscope using a Hamamatsu ORCA CCD camera. For histological analysis of in vivo studies, the fixed brains were sectioned into 60 m thick slices using vibratome (Leica VT1200S) and stored in PBS with 0.02% NaN3 afterwards for up to 1 week. For immunocytochemistry, slices were permeabilized and blocked using 0.3% Triton X-100 in PBS with 1% BSA for 2 hrs, and incubated with the primary antibodies diluted in the same blocking buffer for 3-5 days at 4° C. Secondary antibody staining was performed the same as on cells. Images were acquired by either Olympus IX81 microscope with the same setting as above, or confocal laser scanning microscope (Olympus FV1000) at 2 m with Z-series. Confocal images were taken under water immersion lenses (10× and 40×) and analyzed using FluoView (Olympus) and Photoshop (Adobe Systems).

TABLE 1
List of primary antibodies with validation information

| | | | | Catalog | Reference Pubmed |
|---|---|---|---|---|---|
| Antibody | Source | Species | Dilution | Number | ID/Validation |
| BLBP | Chemicon | rabbit | 1:2000 | AB9558 | PMID: 18983967, 23395372 |
| BRN2 | Santa | goat | 1:500 | Sc-6029 | PMID: 26043730, 23459943, |
| | Cruz | | | | 22566684, |
| | | | | | http://1degreebio.org/reagents/product/18 |
| | | | | | 4322/?gid=1498191 |
| BRN3A | Chemicon | mouse | 1:200 | MAB1585 | PMID: 22750882 |
| Calretinin/CALB2 | Swant | mouse | 1:1000 | 6B3 | PMID: 23642365, 18716623 |
| Calbindin/CALB1 | Swant | mouse | 1:1000 | 300 | PMID: 23642365, 21725324 |
| Cleaved | Cell | rabbit | 1:100 | 9661 | https://www.cellsional.com/products/primary- |
| Caspase 3 | Signaling | | | | antibodies/cleaved-caspase-3-asp175- |
| | | | | | antibody/9661, |
| | | | | | http://www.biocompare.com/9776- |
| | | | | | Antibodies/154962-Cleaved-Caspase3- |
| | | | | | Asp175-Antibody/ |
| CTIP2 | Abcam | rat | 1:500 | ab18465 | PMID: 18983967, 23395372, |
| | | | | | 23642365 |
| CUX1 | Santa | rabbit | 1:100 | Sc-13024 | PMID: 18983967, 26752160, |
| | Cruz | | | | 25788693, 25404384 |
| CUX2 | Abcam | rabbit | 1:200 | ab130395 | http://www.abcam.com/cux2- |
| | | | | | antibody-ab130395.html |
| EMX2 | Chemicon | rabbit | 1:100 | AB5730 | PMID: 12644247 |
| FOXG1/BF1 | Stem | rabbit | 1:1000 | NC-FAB | (vendor discontinued). |
| | Culture | | | | Alternative antibody: Cat# M227; |
| | | | | | Takara (Clontech) |
| FOXP2 | Abcam | rabbit | 1:1000 | ab16046 | PMID: 23395372 |
| GABA | Sigma- | rabbit | 1:500 | A2052 | http://1degreebio.org/reagents/product/82 |
| | Aldrich | | | | 8949/?gid=1498385 |
| GAD65/67 | Chemicon | rabbit | 1:500 | AB1511 | https.//www.labome.com/product/EMD- |
| | | | | | Millipore/AB1511.html |
| GFP | Abcam | chicken | 1:1000 | ab13970 | http://www.biocompare.com/9776- |
| | | | | | Antibodies/103193-GFP- |
| | | | | | antibody/#productdetails |
| | Nacalai | rat | 1:1000 | 04404-84 | http://www.nacalai.co.ip/global/reagent/An |
| | Tesque Inc. | | | | tibody_Reference_List/Anti-GFP_Rat.html |
| | Invitrogen | rabbit | 1:1000 | A11122 | http://1degreebio.org/reagents/product/86 |
| | | | | | 8907/?gid=0 |
| ISL1 | DSHB | mouse | 1:200 | 39.4D5-c | https://www.labome.com/product/Developmental- |
| | | | | | Studies-Hybridoma-Bank/39- |
| | | | | | 4D5.html |
| MAP2ab | Sigma- | mouse | 1:1000 | M1406 | https://www.labome.com/product/Sigma- |
| | Aldrich | | | | Aldrich/M1406.html |
| NKX2.1 | ThermoFisher | mouse | 1:200 | MS-699-P1 | https://www.thermofisher.com/order/catalo |
| | Scientific | | | | g/product/MS-699-B |
| PAX6 | Covance | rabbit | 1:100 | PRB-278P | http://1degreebio.org/reagents/product/75 |
| | | | | | 1403/?gid=0 |
| pH3 | Cell | rabbit | 1:100 | 9701 | https:///www.citeab.com/antibodies/126321- |
| | Signaling | | | | 9701-phospho-histone-h3-ser10- |
| | | | | | antibody?utm_campaign=Widget+Ali+Cita |
| | | | | | tions&utm_medium=Widget&utm_source= |
| | | | | | Cell+Signaling+Technology |
| REELIN | Chemicon | mouse | 1:200 | MAB5364 | https://www.labome.com/product/EMD- |
| | | | | | Millipore/MAB5366.html |
| RGS4 | Santa | goat | 1:100 | Sc-6203 | PMID: 21798518, 11463389 |
| | Cruz | | | | |
| SC121 | StemCells | mouse | 1:1000 | AB-121- | PMID: 15280535, 16172374, |
| | | | | U-050 | 19733542, 17044030 |
| SATB2 | Abcam | mouse | 1:50 | ab51502 | PMID: 18983967, |
| | | | | | https://www.labome.com/product/Abcam/a |
| | | | | | b51502.html |
| | Abcam | rabbit | 1:1000 | ab34735 | PMID: 23395372, |
| | | | | | https://www.labome.com/product/Abc |
| | | | | | am/ab34735.html |
| SIX1 | Sigma- | rabbit | 1:500 | HPA001893 | https://www.labome.com/review/gene/hum |
| | Aldrich | | | | an/SIX1-antibody.html |
| SOX10 | Santa | goat | 1:100 | Sc-17342 | https://www.scbt.com/scbt/product/sox-10- |
| | | | | | antibody-n-20?productCanUrl=sox-10- |
| | Cruz | | | | antibody-n-20&_requestid=2038490 |
| Human-specific | Enzo Life | mouse | 1:1000 | ADI-905- | PMID: 26863197, |
| Synaptophysin | Sciences | | | 782-100 | https://www.antibodypedia.com/gene/501/ |
| | | | | | SYP/antibody/643938/ADI-905-782-100 |
| TBR1 | Millipore | rabbit | 1:500 | AB9616 | http://www.abcam.com/tbr1-antibody- |
| | | | | | ab31940-references.html |
| TBR2 | Chemicon | rabbit | 1:1000 | AB2283 | https://www.labome.com/product/EMD- |
| | | | | | Millipore/AB2283.html |
| TLE4 | Santa | mouse | 1:200 | Sc-365406 | https://www.scbt.com/scbt/product/tle4- |
| | Cruz | | | | antibody-m-200 |
| TUJ1 | Covance | rabbit | 1:2500 | MRB-435P | http://www.biolegend.com/purified-anti- |
| | | | | | tubulin-beta-3-tubb3-antibody-11579.html |
| | Covance | mouse | 1:2500 | MMS-435P | http://www.biolegend.com/purified-anti- |
| | | | | | tubulin-beta-3-tubb3-antibody-11580.html |

iDISCO Whole Brain Immunofluorescence and Imaging

Brains were processed as described in the iDISCO protocol¹⁸, with modifications described in the updated online protocol (http://idisco.info, January 2015 version). The primary antibodies used were chicken anti-GFP (1:1000; Ayes GFP-1020), and mouse anti-hSynaptophysin (1:1000; Enzo Life Science). Secondary antibodies used were donkey anti-chicken-Alexa647 (1:1000; Jackson Jmmunoresearch) and donkey anti-mouse-Alexa568 (1:1000; Life Technologies). The cleared samples were imaged on a light sheet microscope (Ultramicroscope II, LaVision Biotec) equipped with a sCMOS camera (Andor Neo) and a 2×/0.5 objective lens equipped with a 6 mm working distance dipping cap.

Flow Cytometry

Cells were disassociated with Accutase for 30 min to 1 hr at 37° C. After washing, cells were resuspended in 1×PBS with propidium iodide (2 μg/ml), and sorted by FACScalibur platform (BD Biosciences). GFP+% was determined within the propidium iodide negative population. For intracellular flow cytometry, cells were disassociated and washed, and fixed with 4% (v/v) paraformaldehyde for 20 min. Fixed cells were then permeabilized and stained using 1×BD Perm/Wash Buffer (BD Biosciences) following the manufacturer's instructions. Primary conjugated antibodies for flow cytometry used were Nestin-Alexa647 (1:50; BD Pharmingen, #560341) and TUJ1-Alexa488 (1:50; BD Pharmingen, #560338). Cells were sorted using FACScalibur. Results were analyzed using FlowJo (Version 7.6).

Electrophysiology

Cells were replated at day 8 of differentiation and maintained on 35 mm diameter petri dishes (Falcon) in neuronal differentiation medium. On day 16, 23, 30, 37 and 40, electrophysiology was performed with pre-incubation in DMEM medium (Life Technology) at 37° C. for 2 hrs before recording. For in vivo recording of EGFP+ grafted cells, NOD-SCID IL2Rgc^{-/-} mice transplanted with EGFP+H9 derived cells were anesthetized with Avertin and decapitated. The brain was removed and 350 m coronal brain slices were sectioned on a Vibratome (Leica Microsystems) in ice-cold choline chloride-based cutting solution containing (in mM): 120 choline chloride, 26 NaHCO₃, 2.6 KCl, 1.25 NaH2PO4, 7 MgSO4, 0.5 CaCl₂), 1.3 ascorbate acid and 15 D-glucose, bubbled with 95% 02 and 5% C02. Slices were transferred into artificial cerebral spinal fluid (ACSF) containing (in mM): 126 NaCl, 3 KCl, 1.2 NaH2PO4, 1.3 MgSO4, 2.4 CaCl₂), 26 NaHCO₃ and 10 D-glucose, bubbled with 95% 02 and 5% C02, and recovered in an interface chamber at 32° C. for at least 1 hr, and then kept at room temperature before being transferred to a recoding chamber containing ACSF at 34° C.

An infrared-DIC microscope (Olympus BX51) equipped with epifluorescence illumination, a CCD camera, and two water immersion lenses (×10 and ×60) were used to visualize and target recording electrodes to EGFP+ grafted cells and H9 derived neurons in vitro. Glass recording electrodes (7-9 MQ resistance) were filled with an intracellular solution consisting of (in mM): 126 potassium-gluconate, 2 KCl, 2 MgCl2, 0.2 EGTA, 10 HEPES, 4 Na2ATP, 0.4 Na2GTP and 0.5% neurobiotin (Invitrogen) (pH 7.25 and 295 mOsm/kg). Recordings data were collected using Multiclamp 700B amplifier and pCLAMP10 software (Molecular Devices). The firing events were picked up and the kinetics of firing was analyzed using Clampfit10.2. The input resistance of a cell at the point of a small hyperpolarization current injection (−5 pA) pulse was given by Ohm's law from the membrane potential change after it has reached plateau. Spontaneous-PSCs were analyzed using mini Analysis Program (Synaptosoft Inc.).

Transplantation into Neonatal Mouse

All procedures were performed following NIH guidelines, and were approved by the local Institutional Animal Care and Use Committee (IACUC), the Institutional Biosafety Committee (IBC) and the Embryonic Stem Cell Research Committee (ESCRO). P1S5D cells were disassociated with Accutase on day 8 of induction and filtered with 40 m cell strainer (Falcon). Cells were washed once and resuspended in ice old PBS at the density of 100,000 cells/μl, and were then taken by a 10 μl syringe (Hamilton) with a 33-gauge sharp needle. A total of 2 μl cells were injected at the speed of 1 l/1 min into the somatosensory cortex of P2 neonatal NOD-SCID IL2Rgc_mice (Jackson Laboratory) with the aid of stereotactic apparatus and electrical pump (Boston Scientific) to drive the syringe. Fully anesthetized mice were transcardially perfused with PBS containing heparin (20 units/ml) at 1 month, 1.5 months, 3 months, and 6 months post grafting, and followed by 20 ml of 4% paraformaldehyde. Mouse brain was then extracted and post-fixed by 4% paraformaldehyde overnight.

### REFERENCES CITED

1. Chambers, S. M. et al. Highly efficient neural conversion of human ES and iPS cells by dual inhibition of SMAD signaling. Nature biotechnology 27, 275-280 (2009).
2. Mica, Y., Lee, G., Chambers, S. M., Tomishima, M. J. & Studer, L. Modeling neural crest induction, melanocyte specification, and disease-related pigmentation defects in hESCs and patient-specific iPSCs. Cell Rep 3, 1140-1152 (2013).
3. Menendez, L., Yatskievych, T. A., Antin, P. B. & Dalton, S. Wnt signaling and a Smad pathway blockade direct the differentiation of human pluripotent stem cells to multipotent neural crest cells. Proceedings of the National Academy of Sciences of the United States of America 108, 19240-19245 (2011).
4. Maroof, A. M. et al. Directed differentiation and functional maturation of cortical interneurons from human embryonic stem cells. Cell Stem Cell 12, 559-572 (2013).
5. Sun, L. et al. Design, synthesis, and evaluations of substituted 3-[(3- or 4-carboxyethylpyrrol-2-yl)methylidenyl]indolin-2-ones as inhibitors of VEGF, FGF, and PDGF receptor tyrosine kinases. Journal of medicinal chemistry 42, 5120-5130 (1999).
6. Dovey, H. F. et al. Functional gamma-secretase inhibitors reduce beta-amyloid peptide levels in brain. Journal of neurochemistry 76, 173-181 (2001).
7. Chambers, S. M. et al. Combined small-molecule inhibition accelerates developmental timing and converts human pluripotent stem cells into nociceptors. Nature biotechnology 30, 715-720 (2012).
8. Barrett, S. D. et al. The discovery of the benzhydroxamate MEK inhibitors CI-1040 and PD 0325901. Bioorganic & medicinal chemistry letters 18, 6501-6504 (2008).
9. Huang, S. M. et al. Tankyrase inhibition stabilizes axin and antagonizes Wnt signalling. Nature 461, 614-620 (2009).
10. Dincer, Z. et al. Specification of functional cranial placode derivatives from human pluripotent stem cells. Cell Rep 5, 1387-1402 (2013).
11. Lanner, F. & Rossant, J. The role of FGF/Erk signaling in pluripotent cells. Development 137, 3351-3360 (2010).
12. Pucilowska, J., Puzerey, P. A., Karlo, J. C., Galan, R. F. & Landreth, G. E. Disrupted ERK signaling during cortical development leads to abnormal progenitor proliferation, neuronal and network excitability and behavior, modeling human neuro-cardio-facial-cutaneous and related syndromes. J Neurosci 32, 8663-8677 (2012).
13. Greber, B. et al. FGF signalling inhibits neural induction in human embryonic stem cells. Embo J 30, 4874-4884 (2011).
14. Greig, L. C., Woodworth, M. B., Galazo, M. J., Padmanabhan, H. & Macklis, J. D. Molecular logic of neocortical projection neuron specification, development and diversity. Nat Rev Neurosci 14, 755-769 (2013).
15. Shi, Y., Kirwan, P., Smith, J., Robinson, H. P. & Livesey, F. J. Human cerebral cortex development from pluripotent stem cells to functional excitatory synapses. Nat Neurosci 15, 477-486, S471 (2012).
16. Lie, D. C. et al. Wnt signalling regulates adult hippocampal neurogenesis. Nature 437, 1370-1375 (2005).
17. Salinas, P. C. Wnt signaling in the vertebrate central nervous system: from axon guidance to synaptic function. Cold Spring Harb Perspect Biol 4 (2012).
18. Renier, N. et al. iDISCO: a simple, rapid method to immunolabel large tissue samples for volume imaging. Cell 159, 896-910 (2014).
19. Elkabetz, Y. et al. Human ES cell-derived neural rosettes reveal a functionally distinct early neural stem cell stage. Genes Dev 22, 152-165 (2008).
20. Michelsen, K. A. et al. Area-specific reestablishment of damaged circuits in the adult cerebral cortex by cortical neurons derived from mouse embryonic stem cells. Neuron 85, 982-997 (2015).
21. Kriks, S. et al. Dopamine neurons derived from human ES cells efficiently engraft in animal models of Parkinson's disease. Nature 480, 547-551 (2011).
22. Grealish, S. et al. Human ESC-derived dopamine neurons show similar preclinical efficacy and potency to fetal neurons when grafted in a rat model of Parkinson's disease. Cell Stem Cell 15, 653-665 (2014).
23. Zhang, Y. et al. Rapid single-step induction of functional neurons from human pluripotent stem cells. Neuron 78, 785-798 (2013).
24. Hoya-Arias, R., Tomishima, M., Perna, F., Voza, F. & Nimer, S. D. L3MBTL1 deficiency directs the differentiation of human embryonic stem cells toward trophectoderm. Stem Cells Dev 20, 1889-1900 (2011).
25. Sanjana, N. E. et al. A transcription activator-like effector toolbox for genome engineering. Nat Protoc 7, 171-192 (2012).
26. Kaech, S. & Banker, G. Culturing hippocampal neurons. Nat Protoc 1, 2406-2415 (2006).
27. Hoya-Arias, R., Tomishima, M., Perna, F., Voza, F. & Nimer, S. D. L3MBTL1 deficiency directs the differentiation of human embryonic stem cells toward trophectoderm. Stem Cells Dev 20, 1889-1900 (2011).
28. Sanjana, N. E. et al. A transcription activator-like effector toolbox for genome engineering. Nat Protoc 7, 171-192 (2012).
29. Kaech, S. & Banker, G. Culturing hippocampal neurons. Nat Protoc 1, 2406-2415 (2006).
30. Espuny-Camacho, I. et al. Pyramidal neurons derived from human pluripotent stem cells integrate efficiently into mouse brain circuits in vivo. Neuron 77, 440-456 (2013).
31. Zimmer, B. et al. Derivation of Diverse Hormone-Releasing Pituitary Cells from Human Pluripotent Stem Cells. Stem Cell Reports 6, 858-872 (2016).
32. Lee, G. et al. Modelling pathogenesis and treatment of familial dysautonomia using patient-specific iPSCs. Nature 461, 402-406 (2009).
33. Bardy, C. et al. Neuronal medium that supports basic synaptic functions and activity of human neurons in vitro. Proceedings of the National Academy of Sciences of the United States of America 112, E2725-2734 (2015).
34. Du, Z. W. et al. Generation and expansion of highly pure motor neuron progenitors from human pluripotent stem cells. Nat Commun 6, 6626 (2015).
35. Maury, Y. et al. Combinatorial analysis of developmental cues efficiently converts human pluripotent stem cells into multiple neuronal subtypes. Nature biotechnology 33, 89-96 (2015).
36. Borghese, L. et al. Inhibition of notch signaling in human embryonic stem cell-derived neural stem cells delays GUS phase transition and accelerates neuronal differentiation in vitro and in vivo. Stem Cells 28, 955-964 (2010).
37. Gao, P., Sultan, K. T., Zhang, X. J. & Shi, S. H. Lineage-dependent circuit assembly in the neocortex. Development 140, 2645-2655 (2013).

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. An in vitro method for differentiating pluripotent stem cells into cortical neuron precursors, comprising exposing stem cells to:
at least one anaplastic lymphoma kinase (ALK) inhibitor, and
at least one inhibitor of wingless (Wnt) signaling mediated by β-catenin; and
exposing the cells to at least one MAPK/ERK kinase inhibitor,
at least one fibroblast growth factor (FGF) inhibitor, and
at least one γ-secretase inhibitor
to obtain a-population of differentiated cells expressing at least one cortical neuron precursor marker selected from the group consisting of:
beta-tubulin III (TUJ1),
transducin-like enhancer protein 4 (TLE4),
T-box brain transcription factor 1 (TBR1),
doublecortin (DCX),
reeling,
COUP-TF-Interacting Protein 2 (CTIP2),
forkhead box P2 (FOXP2),
special AT-rich sequence-binding protein 2 (SATB2),
regulator of G protein signaling 4 (RGS4),
cut like homeobox 2 (CUX2),
brain lipid binding protein (BLBP), and
combinations thereof.

2. The method of claim 1, wherein the method further comprises obtaining a population of cells expressing paired box 6 (PAX6) at least 6 days from initial exposure of the stem cells to the at least one ALK inhibitor.

3. The method of claim 1, wherein the method further comprises obtaining a population of cells expressing PAX6 up to about 6 days from initial exposure of the stem cells to the at least one ALK inhibitor.

4. The method of claim 1, wherein the at least one MAPK/ERK kinase inhibitor, at least one FGF inhibitor, and at least one γ-secretase inhibitor are exposed to the cells at least 2 or 3 days from initial exposure of the stem cells to the at least one ALK inhibitor.

5. The method of claim 1, wherein the at least one MAPK/ERK kinase inhibitor, at least one FGF inhibitor, and at least one γ-secretase inhibitor are exposed to the cells up to about 2 or about 3 days from initial exposure of the stem cells to the at least one ALK inhibitor.

6. The method of claim 1, wherein the differentiated cells express the at least one marker at least 13 days from initial exposure of the stem cells to the at least one ALK inhibitor.

7. The method of claim 1, wherein the differentiated cells express the at least one marker up to about 13 days from initial exposure of the stem cells to the at least one ALK inhibitor.

8. The method of claim 1, wherein at least 50% of the differentiated cells express TUJ1 and further express TBR1, TLE4, or a combination thereof.

9. The method of claim 6, wherein the cells exhibit electrophysiological activity of differentiated cortical neurons at least 16 days from initial exposure of the stem cells to the at least one ALK inhibitor.

10. The method of claim 1, wherein the at least one ALK inhibitor, and at least one inhibitor of Wnt signaling are exposed to the stem cells for at least 6 days.

11. The method of claim 1, wherein the at least one ALK inhibitor, and at least one inhibitor of Wnt signaling are exposed to the stem cells for up to about 6 days.

12. The method of claim 1, wherein the at least one ALK inhibitor, and at least one inhibitor of Wnt signaling are exposed to the stem cells for at least 7 days.

13. The method of claim 1, wherein the at least one ALK inhibitor, and at least one inhibitor of Wnt signaling are exposed to the stem cells for up to about 7 days.

14. The method of claim 1, wherein the at least one ALK inhibitor comprises 4-[4-(1,3-benzodioxol-5-yl)-5-(2-pyridinyl)-1H-imidazol-2-yl]-benzamide (SB431542), 4-(6-(4-(piperazin-1-yl)phenyl)pyrazolo[1,5-a]pyrimidin-3-yl)quinoline (LDN193189), and mixtures thereof.

15. The method of claim 1, wherein the at least one inhibitor of Wnt signaling comprises 3,5,7,8-tetrahydro-2-[4-(trifluoromethyl)phenyl]-4H-thiopyrano[4,3-d]pyrimidin-4-one (XAV939).

16. The method of claim 1, wherein the at least one MAPK/ERK kinase inhibitor comprises N-(2,3-dihydroxy-propoxy)-3,4-difluoro-2-(2-fluoro-4-iodo-phenylamino)-benzamide (PD0325901).

17. The method of claim 1, wherein the at least one FGF inhibitor comprises 2-[(1,2-dihydro-2-oxo-3H-indol-3-ylidene)methyl]-4-methyl-1H-pyrrole-3-propanoic acid (SU5402).

18. The method of claim 1, wherein the γ-secretase inhibitor comprises tert-butyl (2S)-2-[[(2S)-2-[[2-(3,5-difluorophenyl)acetyl]amino]propanoyl]amino]-2-phenylacetate (DAPT).

19. The method of claim 1, wherein the stem cells are selected from the group consisting of human embryonic stem cells, human induced pluripotent stem cells, human parthenogenetic stem cells, primordial germ cell-like pluripotent stem cells, epiblast stem cells, and F-class pluripotent stem cells.

20. The method of claim 1, wherein the method further comprises subjecting the differentiated cells to conditions inducing maturation of the differentiated cells into cortical neurons, wherein the conditions comprises exposing the differentiated cells to at least one-compound that is capable of activating at least one of BDNF signaling, cAMP signaling, and ascorbic acid signaling.
